(19) United States
(12) Patent Application Publication
(10) Pub. No.: US 2025/0161281 A1
(43) Pub. Date: May 22, 2025

(54) EMOPAMIL-BINDING PROTEIN INHIBITORS AND USES THEREOF

(71) Applicant: BIOGEN MA INC., Cambridge, MA (US)

(72) Inventors: Felix Gonzalez Lopez de Turiso, Cambridge, MA (US); Martin Himmelbauer, Cambridge, MA (US); Zhili Xin, Cambridge, MA (US); Nupur Bansal, Medfield, MA (US); TeYu Chen, Charlestown, MA (US); John Howard Jones, Framingham, MA (US); Edward Yin Shiang Lin, Ashland, MA (US)

(21) Appl. No.: 18/832,691

(22) PCT Filed: Jan. 23, 2023
(86) PCT No.: PCT/US2023/011327

§ 371 Date: Jul. 24, 2024

Related U.S. Application Data
(60) Provisional application No. 63/302,195, filed on Jan. 24, 2022.

(51) Int. Cl. A61K 31/439; C07D 451/04

(52) U.S. Cl. CPC A61K 31/439; C07D 451/04

(57) ABSTRACT

Provided are compounds of the Formula (I): (I), or pharmaceutically acceptable salts thereof, which are useful for the inhibition of EBP and in the treatment of a variety of EBP mediated conditions or diseases, such as multiple sclerosis.

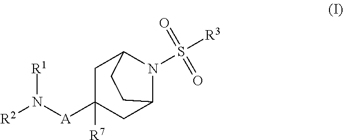

### RELATED APPLICATION

This application claims the benefit of the filing date, under 35 U.S.C. § 119 (e), of U.S. Provisional Application No. 63/302,195, filed on Jan. 24, 2022, the entire contents of which are incorporated herein by reference.

### FIELD OF THE INVENTION

The present disclosure relates to inhibitors of Emopamil-Binding Protein (EBP), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the EBP inhibitor to a warm-blooded animal, especially a human.

### BACKGROUND OF THE INVENTION

Emopamil-Binding Protein (EBP) is a Δ8-Δ7 sterol isomerase enzyme which isomerizes the double bond in sterol molecules, moving the double bond from the 8-9 position to the 7-8 position. Specifically, EBP converts either zymostenol to lathosterol, or zymosterol to dehydrolathosterol, during the biosynthesis of cholesterol (Silve et al., 1996, J Biol Chem. 271 (37), 22434-22440). It has been shown that an accumulation of 8-9 unsaturated sterols activates oligodendrocyte formation and remyelination (Hubler et al., 2019, Nature 560 (7718), 372-376).

Myelin is lipid-based molecule which forms protective layers (myelin sheathes) around nerve cell axons and insulates the axons. Demyelinating diseases, or myelin-related diseases, are a result of these myelin sheathes being damaged, degraded, or reduced in thickness. The loss of the myelin sheathes disrupts the electronic signals from the brain and can lead to nerve damage, vision loss, numbness, muscle weakness, cognitive decline, loss of motor functions, and other similar symptoms. In some myelin-related diseases, such as multiple sclerosis, a subject's immune system targets and breaks down their own myelin sheathes. The ability to repair and regenerate the myelin sheathes is key to treating these myelin-related diseases. Due to its function converting 8-9 sterols, inhibition of EBP is a potential target for activating remyelination, as its inhibition leads to an increase of these 8-9 sterol starting materials (Theodoropoulous et al, 2020, J. Am. Chem. Soc., 142, (13), 6128-6138).

In addition to its role in remyeliniation, EBP has also been shown to be a key enzyme in certain colorectal cancers due to the reduction in essential lipids such as cholesterol (Theodoropoulous et al, 2020, J. Am. Chem. Soc., 142, (13), 6128-6138).

Thus, there is a need for EBP inhibitors as potential therapeutic agents for treating diseases or disorders that are responsive to EBP inhibition.

### SUMMARY OF THE INVENTION

The present disclosure provides compounds that are EBP inhibitors. In a first aspect, the present disclosure relates to compounds having the Formula I:

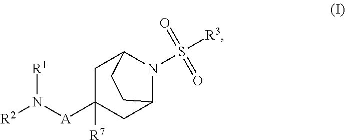

or a pharmaceutically acceptable salt thereof, wherein:
R¹ and R² are each independently selected from H, C₁₋₆alkyl, C₃₋₈cycloalkyl, Het, —Z-Het and Z—C₃₋₈cycloallkyl, wherein the C₁₋₆alkyl, C₃₋₈cycloalkyl and Het are each optionally substituted with one or more R⁴, provided at least one of R¹ and R² is not H; or
R¹ and R² together with the nitrogen atom from which they are attached form a 4 to 10 membered heterocyclyl optionally substituted with one or more substituent R⁴;
Z is C₁₋₄alkyl optionally substituted with one or more halo
Het is 4 to 7 membered monocyclic heterocyclyl or 6 to 10 membered bicyclic heterocyclyl, each of which is optionally substituted with R⁴;
R³ is phenyl, 5 or 6-membered monocyclic heteroaryl, or 9 or 10-membered bicyclic heteroaryl, wherein the phenyl, 5 or 6-membered monocyclic heteroaryl, and 9 or 10-membered bicyclic heteroaryl are each optionally substituted with one or more substituent R⁵;
R⁴, for each occurrence, is independently C₁₋₆alkyl, halo, and —OR^{4a}, wherein the C₁₋₆alkyl is optionally substituted with —OR^{4a};
R^{4a} is H, C₁₋₆alkyl or C₁₋₆haloalkyl;
R⁵, for each occurrence, is independently, halo, —OR^{5a}, C₁₋₆alkyl, C₃₋₆cycloalkyl, 4 to 7-membered monocyclic heterocyclyl, 5- to 6-membered monocyclic heteroaryl, or phenyl, wherein the C₁₋₆alkyl, C₃₋₆cycloalkyl and 5- to 6-membered monocyclic heteroaryl are each optionally substituted with one or more halo, C₁₋₄alkyl or —OR^{5a};
or two R⁵ together with their intervening atoms form a 5 to 7-membered heterocycle optionally substituted with one or more R⁶ independently selected from C₁₋₆alkyl, oxo, halo, and —OR^{5a};
R^{5a} is H, C₁₋₆alkyl or C₁₋₆haloalkyl, wherein the C₁₋₆alkyl is optionally substituted with alkoxy;
R⁷ is H or OR^{7a};
R^{7a} is H, C₁₋₆alkyl or C₁₋₆haloalkyl, wherein the C₁₋₆alkyl is optionally substituted with alkoxy.

Another aspect of the disclosure relates to pharmaceutical compositions comprising compounds of Formula (I) or pharmaceutically acceptable salts thereof, and a pharmaceutical carrier.

In yet another aspect, the present disclosure provides a method of treating a disease or disorder that is responsive to inhibition of EBP in a subject comprising administering to said subject an effective amount of at least one compound described herein or a pharmaceutically acceptable salt thereof. In some embodiments, the present disclosure provides a method for treating multiple sclerosis. In some embodiments, the present disclosure provides a method for promoting myelination in a subject with a myelin-related disorder.

Another aspect of the present disclosure relates to the use of at least one compound described herein or a pharmaceutically acceptable salt thereof, for the manufacture of a medicament for the treatment of a disease or disorder responsive to inhibition of EBP. Also provided is a compound described herein or a pharmaceutically acceptable salt thereof for use in treating a disease or disorder responsive to inhibition of EBP.

### DETAILED DESCRIPTION OF THE INVENTION

The present disclosure provides compounds and pharmaceutical compositions thereof that may be useful in the treatment of diseases or disorders through mediation of EBP function/activity, such as multiple sclerosis or other myelin-related disorders. In some embodiments, the compounds of present disclosure are EBP inhibitors.

### Compounds and Compositions

In a first embodiment, the present disclosure provides a compound of Formula (I):

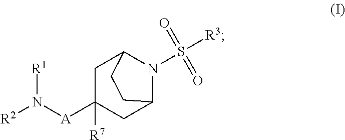

or a pharmaceutically acceptable salt thereof, wherein:
R¹ and R² are each independently selected from H, C₁₋₆alkyl, C₃₋₈cycloalkyl, Het, —Z-Het, wherein the C₁₋₆alkyl, C₃₋₈cycloalkyl and Het are each optionally substituted with one or more R⁴, provided at least one of R¹ and R² is not H; or
R¹ and R² together with the nitrogen atom from which they are attached form a 4 to 10 membered heterocyclyl optionally substituted with one or more substituent R⁴;
Z is C₁₋₄alkyl optionally substituted with one or more halo;
Het is 4 to 7 membered monocyclic heterocyclyl or 6 to 10 membered bicyclic heterocyclyl, each of which is optionally substituted with R⁴;
R³ is phenyl, 5 or 6-membered monocyclic heteroaryl, or 9 or 10-membered bicyclic heteroaryl, wherein the phenyl, 5 or 6-membered monocyclic heteroaryl, and 9 or 10-membered bicyclic heteroaryl are each optionally substituted with one or more substituent R⁵;
R⁴, for each occurrence, is independently C₁₋₆alkyl, halo, and —OR^{4a};
R^{4a} is H, C₁₋₆alkyl or C₁₋₆haloalkyl;
R⁵, for each occurrence, is independently, halo, —OR^{5a}, C₁₋₆alkyl, C₃₋₆cycloalkyl, 4 to 7-membered monocyclic heterocyclyl, or phenyl, wherein the C₁₋₆alkyl and C₃₋₆cycloalkyl are each optionally substituted with one or more halo, —OR^{5a};
or two R⁵ together with their intervening atoms form a 5 to 7-membered heterocycle optionally substituted with one or more R⁶ independently selected from C₁₋₆alkyl, oxo, halo, and —OR^{5a};
R^{5a} is H, C₁₋₆alkyl or C₁₋₆haloalkyl, wherein the C₁₋₆alkyl is optionally substituted with alkoxy;
R⁷ is H or OR^{7a};
R^{7a} is H, C₁₋₆alkyl or C₁₋₆haloalkyl, wherein the C₁₋₆alkyl is optionally substituted with alkoxy.

In a second embodiment, the compound of the present disclosure is represented by Formula (II):

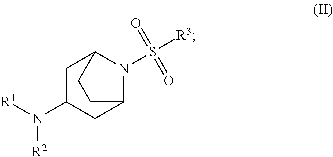

or a pharmaceutically acceptable salt thereof, wherein the variables in Formula (II) are as defined in the first aspect or the first embodiment above.

In a third embodiment, the compound of the present disclosure is represented by Formula (IIA) or (IIB):

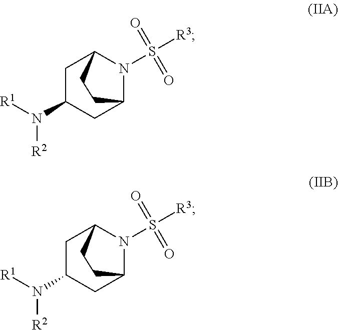

or a pharmaceutically acceptable salt thereof, wherein the variables in Formula (IIA) and (IIB) are as defined in the first aspect or the first or second embodiment above.

In a fourth embodiment, for the compounds of Formula (I), (II), (IIA), or (IIB), or a pharmaceutically acceptable salt thereof, R³ is selected from the group consisting of phenyl, pyrazolyl, imidazolyl, oxazolyl, isoxazolyl, pyridinyl, pyrazolopyridinyl, pyrazolopyridinyl, benzotriazolyl, furanyl, oxadiazolyl, imidazopyridinyl, thiophenyl, thiazoyl, triazoyl, and indoyl, each of which is optionally substituted with one to three R⁵; and the remaining variables are as described in the first aspect or the first, second, or third embodiment. In an alternative embodiment, for the compounds of Formula (I), (II), (IIA), or (IIB), or a pharmaceutically acceptable salt thereof, R³ is selected from the group consisting of phenyl, pyrazolyl, imidazolyl, oxazolyl, pyridinyl, pyrazolopyridinyl, pyrazolopyridinyl, benzotriazolyl, furanyl, oxadiazolyl, imidazopyridinyl, and thiophenyl, each of which is optionally substituted with one to three R⁵; and the remaining variables are as described in the first aspect or the first, second, or third embodiment.

In a fifth embodiment, for the compounds of Formula (I), (II), (IIA), or (IIB), or a pharmaceutically acceptable salt thereof, R³ is represented by the following formula:

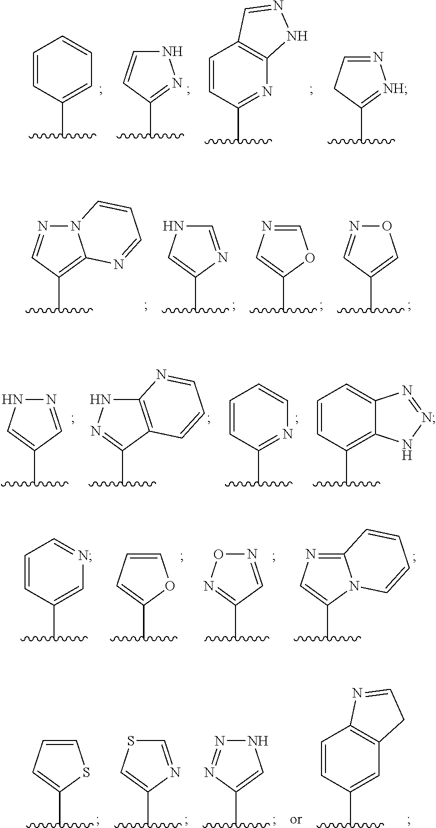

each of which is optionally substituted with one to three R⁵; or R³ is presented by the following formula:

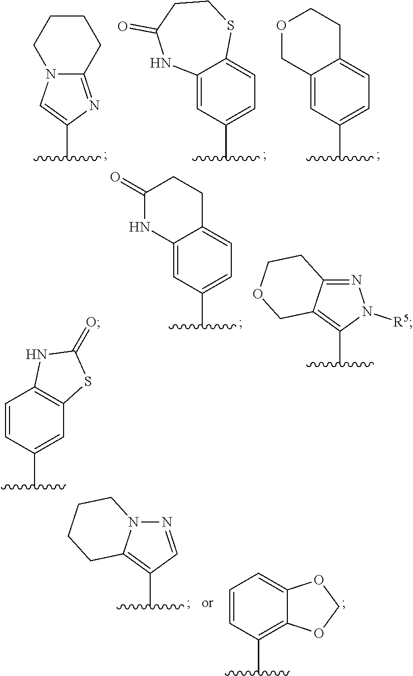

each of which is optionally substituted with one or two R⁶; and the remaining variables are as described in the first aspect or the first, second, third, or fourth embodiment or any alternative embodiments described therein. In an alternative embodiment, for the compounds of Formula (I), (II), (IIA), or (IIB), or a pharmaceutically acceptable salt thereof, R³ is represented by the following formula:

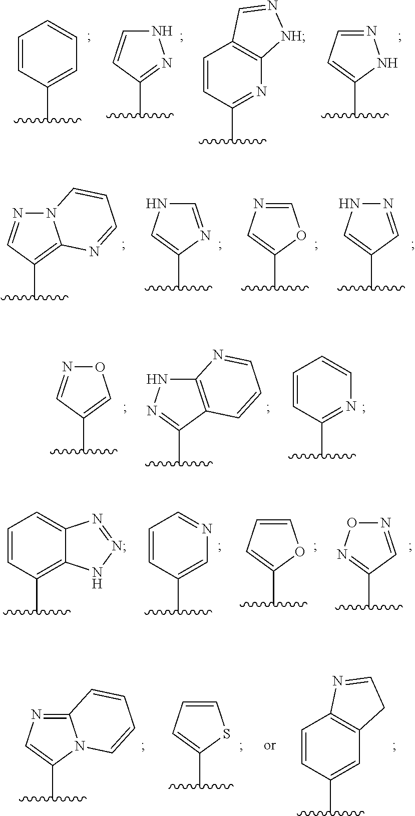

each of which is optionally substituted with one to three R⁵; or R³ is presented by the following formula:

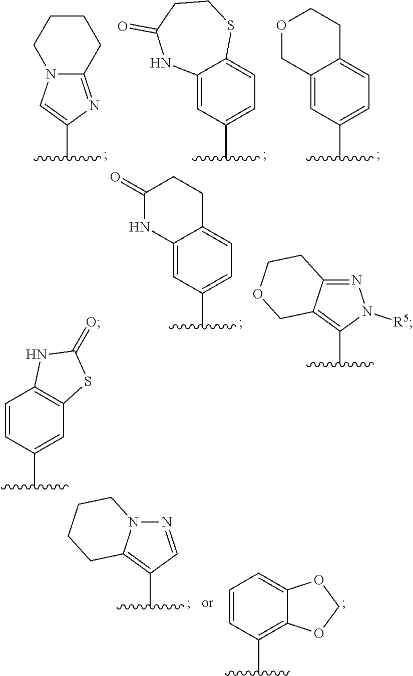

each of which is optionally substituted with one or two R⁶; and the remaining variables are as described in the first aspect or the first, second, third, or fourth embodiment or any alternative embodiments described therein.

In a sixth embodiment, for the compounds of Formula (I), (II), (IIA), or (IIB), or a pharmaceutically acceptable salt thereof, R³ is selected from the group consisting of phenyl, pyrazolyl, imidazolyl, oxazolyl, and pyridinyl, each of which is optionally substituted with one to three R⁵; and the remaining variables are as described in the fourth embodiment.

In a seventh embodiment, for the compounds of Formula (I), (II), (IIA), or (IIB), or a pharmaceutically acceptable salt thereof, R³ is represented by the following formula:

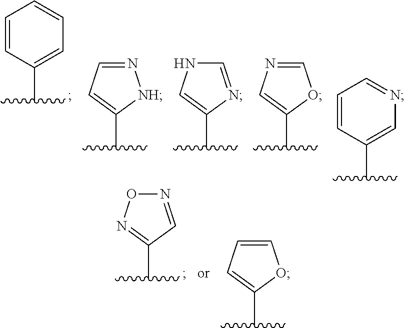

each of which is optionally substituted with one or two R⁵; or R³ is presented by the following formula:

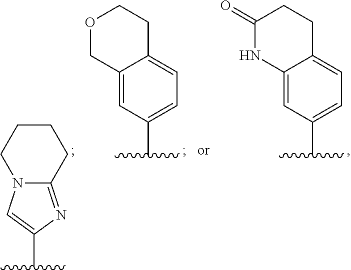 [SMILES: *c1cn2c(n1)CCCC2 ; *c1ccc2c(c1)COCC2 ; *c1ccc2c(c1)NC(=O)CC2]

each of which is optionally substituted with one or two R⁶; and the remaining variables are as described in the fifth embodiment.

In an eighth embodiment, for the compounds of Formula (I), (II), (IIA), or (IIB), or a pharmaceutically acceptable salt thereof, R³ is represented by the following formula:

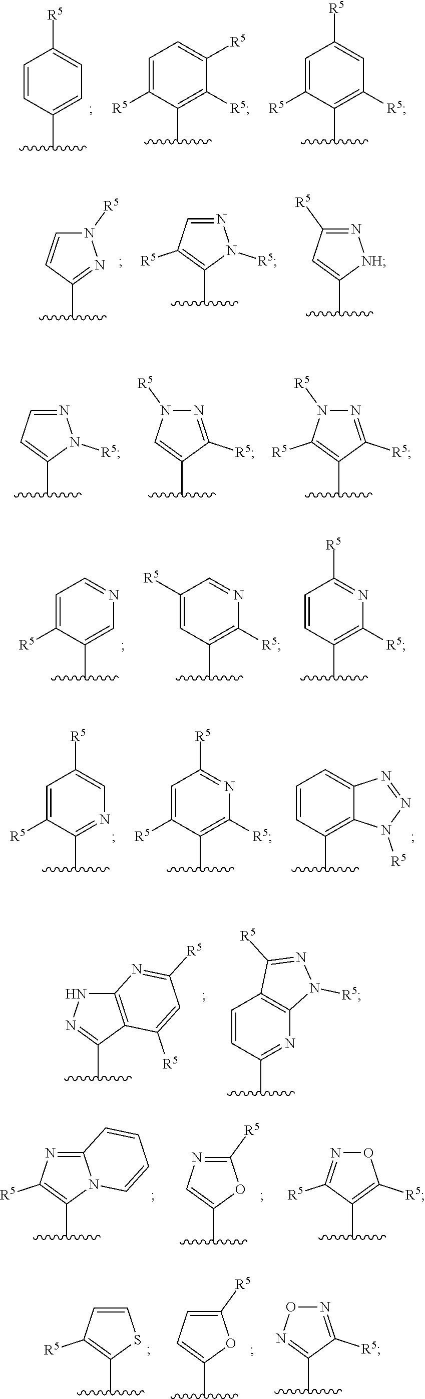

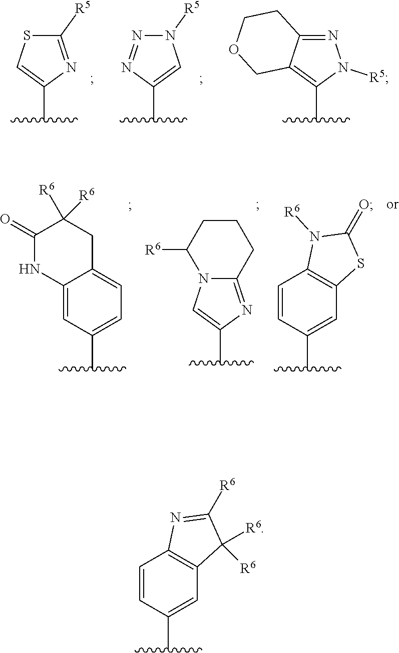

and the remaining variables are as described in the fifth embodiment. In an alternative embodiment, for the compounds of Formula (I), (II), (IIA), or (IIB), or a pharmaceutically acceptable salt thereof. R³ is represented by the following formula:

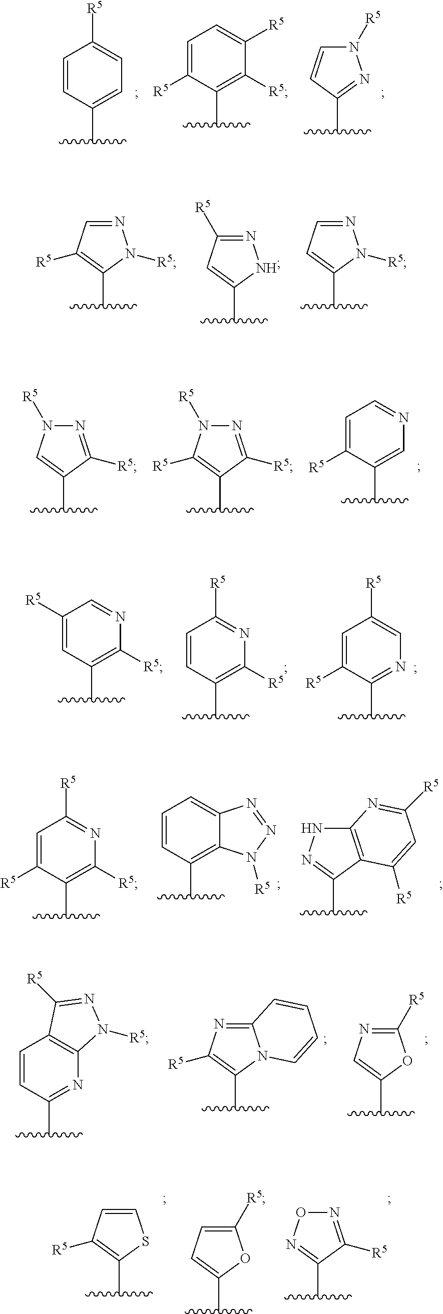

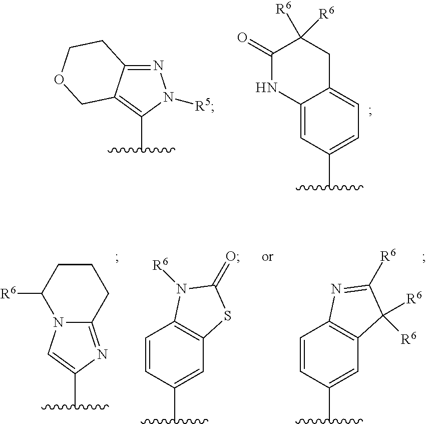

and the remaining variables are as described in the fifth embodiment.

In a ninth embodiment, for the compounds of Formula (I), (II), (IIA), or (IIB), or a pharmaceutically acceptable salt thereof, R³ is represented by the following formula:

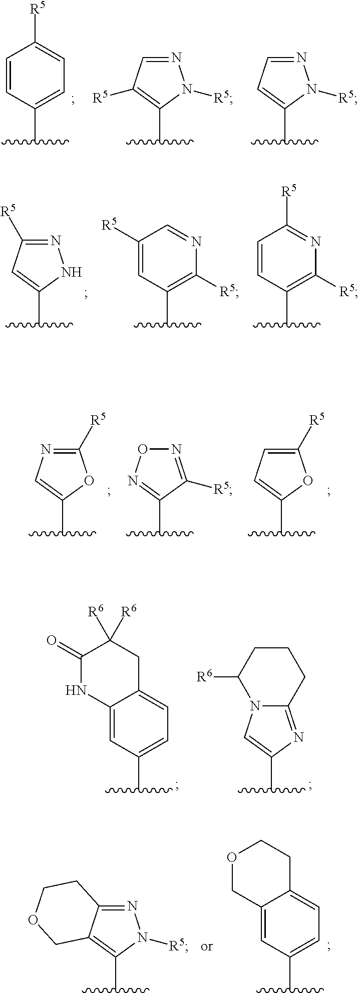

and the remaining variables are as described in the fifth embodiment.

In a tenth embodiment, for the compounds of Formula (I), (II), (IIA), or (IIB), or a pharmaceutically acceptable salt thereof, wherein R⁵, for each occurrence, is independently halo, —OR^{5a}, C₁₋₆alkyl, 4 to 7-membered monocyclic heterocyclyl, or phenyl, wherein the C₁₋₆alkyl is optionally substituted with —OR^{5a} or halo, and wherein the 5- to 6-membered monocyclic heteroaryl is optionally substituted with C₁₋₃alkyl; and R⁶ is C₁₋₃alkyl; and the remaining variables are as described in the first aspect or the first, second, third, fourth, fifth, sixth, seventh, eighth, or ninth embodiment or any alternative embodiments described therein. In an alternative embodiment, for the compounds of Formula (I), (II), (IIA), or (IIB), or a pharmaceutically acceptable salt thereof, wherein R⁵, for each occurrence, is independently halo, —OR^{5a}, C₁₋₆alkyl, 4 to 7-membered monocyclic heterocyclyl, or phenyl, and wherein the C₁₋₆alkyl are each optionally substituted with —OR^{5a}; and R⁶ is C₁₋₃alkyl; and the remaining variables are as described in the first, second, third, fourth, fifth, sixth, seventh, eighth, or ninth embodiment. In a further alternative embodiment, R⁵, for each occurrence, is independently —F, —Cl, —CH₃, —CH₂CH₃, —CF₃, —OCHF₂, —OCH₃, —CH₂OCH₃, tetrahydropyranyl, phenyl, pyridinyl; and R⁶ is —CH₃, optionally, wherein R⁵ is represented by the following formula:

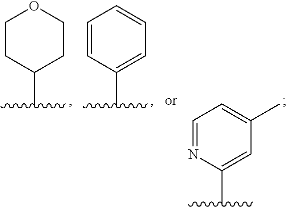

and the remaining variables are as described in the first aspect or the first, second, third, fourth, fifth, sixth, seventh, eighth, or ninth embodiment or any alternative embodiments described therein.

In an eleventh embodiment, for the compounds of Formula (I), (II), (IIA), or (IIB), or a pharmaceutically acceptable salt thereof, wherein R¹ and R² together with the nitrogen atom from which they are attached form a 4 to 9 membered heterocyclyl optionally substituted with one to three R⁴; and the remaining variables are as described in the first aspect or the first, second, third, fourth, fifth, sixth, seventh, eighth, ninth, or tenth embodiment or any alternative embodiments described therein.

In a twelfth embodiment, for the compounds of Formula (I), (II), (IIA), or (IIB), or a pharmaceutically acceptable salt thereof, R¹ and R² together with the nitrogen atom from which they are attached form a 4 to 6-membered monocyclic saturated heteterocyclyl or a 8 to 9 membered bicyclic heterocyclyl, each of which is optionally substituted with one to three R⁴; and the remaining variables are as described in the first aspect or the first, second, third, fourth, fifth, sixth, seventh, eighth, ninth, or tenth embodiment or any alternative embodiments described therein.

In a thirteenth embodiment, for the compounds of Formula (I), (II), (IIA), or (IIB), or a pharmaceutically acceptable salt thereof, R¹ and R² together with the nitrogen atom from which they are attached form a group represented by the following formula:

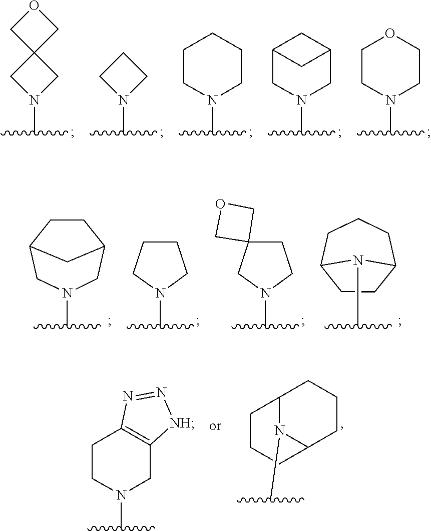

each of which is optionally substituted with one to three R⁴; and the remaining variables are as described in the twelfth embodiment or any alternative embodiments described therein. In an alternative embodiment, for the compounds of Formula (I), (II), (IIA), or (IIB), or a pharmaceutically acceptable salt thereof, R¹ and R² together with the nitrogen atom from which they are attached form a group represented by the following formula:

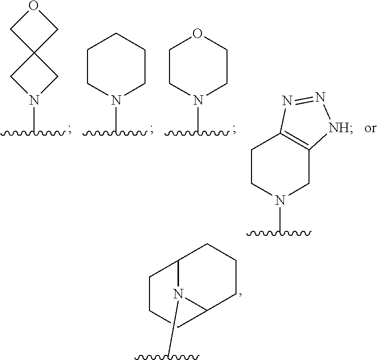

each of which is optionally substituted with one to three R⁴; and the remaining variables are as described in the twelfth embodiment or any alternative embodiments described therein.

In a fourteenth embodiment, for the compounds of Formula (I), (II), (IIA), or (IIB), or a pharmaceutically acceptable salt thereof, wherein R¹ and R² together with the nitrogen atom from which they are attached form a group represented by the following formula:

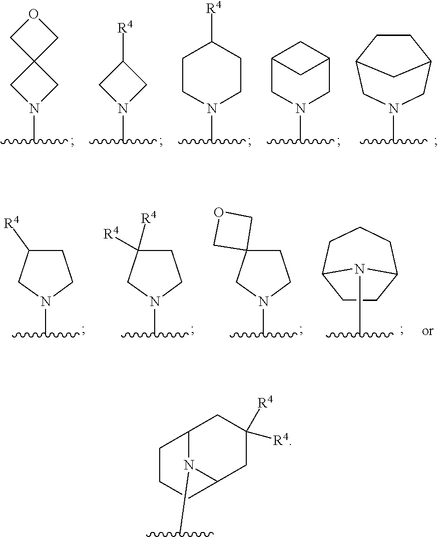

and the remaining variables are as described in the thirteenth embodiment or any alternative embodiments described therein. In an alternative embodiment, for the compounds of Formula (I), (II), (IIA), or (IIB), or a pharmaceutically acceptable salt thereof, wherein R¹ and R² together with the nitrogen atom from which they are attached form a group represented by the following formula:

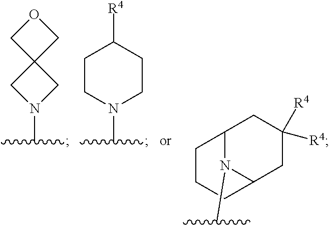

and the remaining variables are as described in the thirteenth embodiment or any alternative embodiments described therein.

In a fifteenth embodiment, for the compounds of Formula (I), (II), (IIA), or (IIB), or a pharmaceutically acceptable salt thereof, R¹ is Het, —C₁₋₃alkyl-Het, —C₁₋₃alkyl-C₃₋₆cyclolkyl, C₃₋₆cycloalkyl or C₁₋₆alkyl, wherein the C₃₋₆cycloalkyl and the C₁₋₆alkyl are each optionally substituted with one or two R⁴; R² is H or C₁₋₃alkyl; Het is a 4 to 6 membered monocyclic saturated heterocyclyl or 6 to 7 membered bicyclic saturated heterocyclyl, wherein the 4 to 6 membered monocyclic saturated heterocyclyl and 6 to 7 membered bicyclic saturated heterocyclyl contain one oxygen ring atom and are each optionally substituted with one or two R⁴; and R⁴, for each occurrence, is independently —OR^{4a}, halo, or C₁₋₃alkyl, wherein the C₁₋₃alkyl is optionally substituted by —OR^{4a}; R^{4a} is H or C₁₋₃alkyl.; and the remaining variables are as described in the first aspect or the first, second, third, fourth, fifth, sixth, seventh, eighth, ninth, or tenth embodiment or any alternative embodiments described therein. In an alternative embodiment, for the compounds of Formula (I), (II), (IIA), or (IIB), or a pharmaceutically acceptable salt thereof, R¹ is Het, —C₁₋₃alkyl-Het, C₃₋₆cycloalkyl or C₁₋₆alkyl, wherein the C₃₋₆cycloalkyl and the C₁₋₆alkyl are each optionally substituted with one or two R⁴; Het is a 4 to 6 membered monocyclic saturated heterocyclyl or 6 to 7 membered bicyclic saturated heterocyclyl, wherein the 4 to 6 membered monocyclic saturated heterocyclyl and 6 to 7 membered bicyclic saturated heterocyclyl contain one oxygen ring atom and are each optionally substituted with one or two R⁴; R⁴, for each occurrence, is independently-OH, halo or C₁₋₃alkyl; R² is H or C₁₋₃alkyl; and the remaining variables are as described in the first aspect or the first, second, third, fourth, fifth, sixth, seventh, eighth, ninth, or tenth embodiment or any alternative embodiments described therein.

In a sixteenth embodiment, for the compounds of Formula (I), (II), (IIA), or (IIB), or a pharmaceutically acceptable salt thereof, Het is oxetanyl, tetrahydro-2H-pyranyl, tetrahydrofuranyl, 2-oxaspiro[3.3]heptanyl, or 5-oxabicyclo[2.1.1]hexanyl, each of which is optionally substituted with one R⁴; and the remaining variables are as described in the fifteenth embodiment or any alternative embodiments described therein. In an alternative embodiment, for the compounds of Formula (I), (II), (IIA), or (IIB), or a pharmaceutically acceptable salt thereof, Het is oxetanyl, tetrahydro-2H-pyranyl, or 5-oxabicyclo[2.1.1]hexanyl, each of which is optionally substituted with one R⁴; and the remaining variables are as described in the fifteenth embodiment or any alternative embodiments described therein.

In a seventeenth embodiment, for the compounds of Formula (I), (II), (IIA), or (IIB), or a pharmaceutically acceptable salt thereof, R¹ is represented by the following formula:

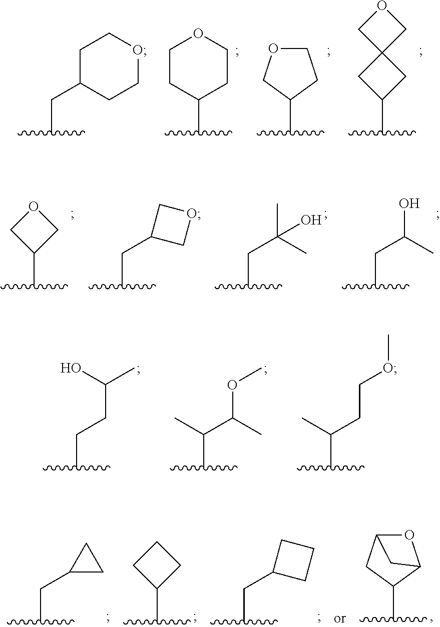

each of which is optionally substituted with one or two R⁴; and the remaining variables are as described in the fifteenth embodiment or any alternative embodiments described therein. In an alternative embodiment, for the compounds of Formula (I), (II), (IIA), or (IIB), or a pharmaceutically acceptable salt thereof, R¹ is represented by the following formula:

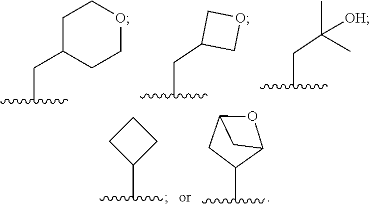

each of which is optionally substituted with one or two R⁴; and the remaining variables are as described in the fifteenth embodiment or any alternative embodiments described therein.

In an eighteenth embodiment, for the compounds of Formula (I), (II), (IIA), or (IIB), or a pharmaceutically acceptable salt thereof, R¹ is represented by the following formula:

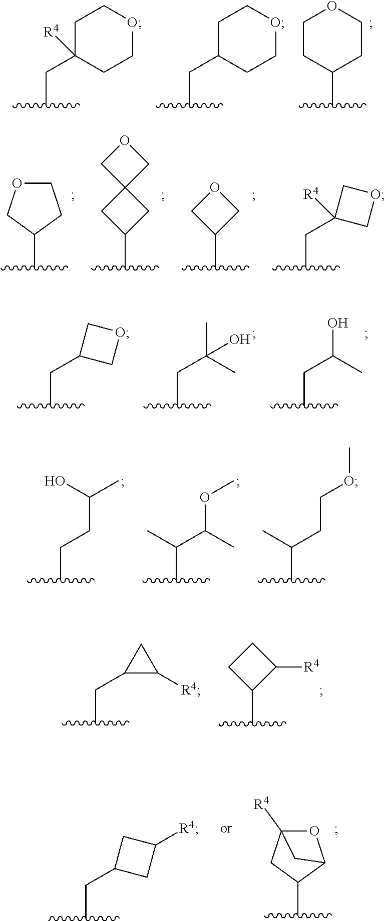

and the remaining variables are as described in the fifteenth embodiment or any alternative embodiments described therein. In an alternative embodiment, for the compounds of Formula (I), (II), (IIA), or (IIB), or a pharmaceutically acceptable salt thereof, R¹ is represented by the following formula:

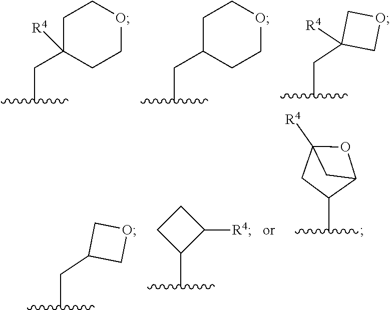

and the remaining variables are as described in the fifteenth embodiment or any alternative embodiments described therein.

In a nineteenth embodiment, for the compounds of Formula (I), (II), (IIA), or (IIB), or a pharmaceutically acceptable salt thereof, R² is H or —CH₃; and the remaining variables are as described in the fifteenth, sixteenth, seventeenth, or eighteenth embodiment or any alternative embodiments described therein. In an alternative embodiment, for the compounds of Formula (I), (II), (IIA), or (IIB), or a pharmaceutically acceptable salt thereof, R² is H; and the remaining variables are as described in the fifteenth, sixteenth, seventeenth, or eighteenth embodiment or any alternative embodiments described therein.

In one embodiment, for the compounds of Formula (I), (II), (IIA), or (IIB), or a pharmaceutically acceptable salt thereof, each R⁴, for each occurrence, is independently —F, —CH₃, —CH₂CH₂OCH₃, —CH₂OH, —OH, or —OCH₃; and the remaining variables are as described in the first aspect or the first, second, third, fourth, fifth, sixth, seventh, eighth, ninth, tenth, fifteenth, sixteenth, seventeenth, eighteenth, or ninteenth embodiment or any alternative embodiments described therein.

In a twentieth embodiment, the compound of the present disclosure is represented by Formulas (III) or (IV):

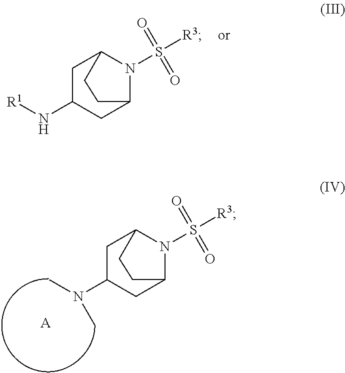

or a pharmaceutically acceptable salt thereof, wherein:
R¹ is C₃₋₆cycloalkyl, Het, or —CH₂-Het, wherein the C₃₋₆cycloalkyl is optionally substituted with one R⁴;
Het is a 4 to 6 membered monocyclic saturated heterocyclyl or a 6 to 7 membered bicyclic saturated heterocyclyl, wherein the 4 to 6 membered monocyclic saturated heterocyclyl and the 6 to 7 membered bicyclic saturated heterocyclyl contain one oxygen ring atom and are each optionally substituted with one R⁴;
Ring A is a 4 to 6 membered monocyclic saturated heterocyclyl or a 8 or 9 membered bicyclic heterocyclyl, each of which is optionally substituted with one or two R⁴;
R³ is phenyl, pyridinyl, or a 5-membered heteroaryl, each of which is optionally substituted with one or two R⁵;
R⁴, for each occurrence, is independently selected from-OH, halo and C₁₋₃alkyl;
R⁵, for each occurrence, is independently selected from halo, —OR^{5a}, C₁₋₆alkyl, 4 to 6-membered monocyclic heterocyclyl, or phenyl, wherein the C₁₋₆alkyl is optionally substituted with one to three halo or —OR^{5a};
or two R⁵ together with their intervening atoms form a 6-membered heterocycle optionally substituted with one to three R⁶ independently selected from C₁₋₆alkyl, oxo, and halo; and
R^{5a} is H, C₁₋₃alkyl, or C₁₋₃haloalkyl; and the remaining variables are as described in the first aspect or the first or second embodiment.

In a twenty-first embodiment, for the compounds of Formula (III) or (IV), or a pharmaceutically acceptable salt thereof, Het is oxetanyl, tetrahydro-2H-pyranyl, or 5-oxabicyclo[2.1.1]hexanyl, each of which is optionally substituted with one R⁴;

Ring A is

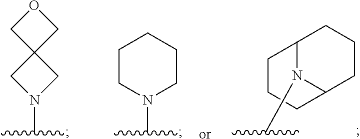

each of which is optionally substituted with one to two R⁴;
R³ is

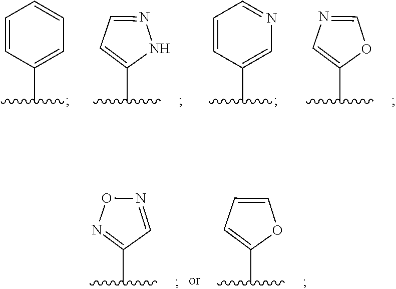

each of which is optionally substituted with one or two R⁵;
or R³ is

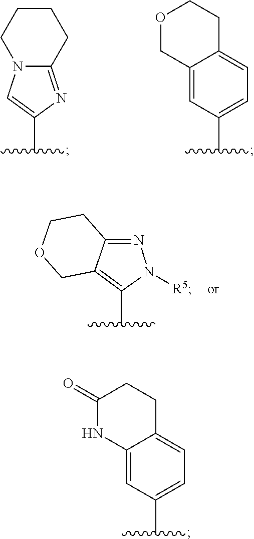

each of which is optionally substituted with one or two R⁶; and the remaining variables are as described in the twentieth embodiment.

In a twenty-second embodiment, for the compounds of the formula (III) or (IV), or a pharmaceutically acceptable salt thereof, R³ is represented by the following formula:

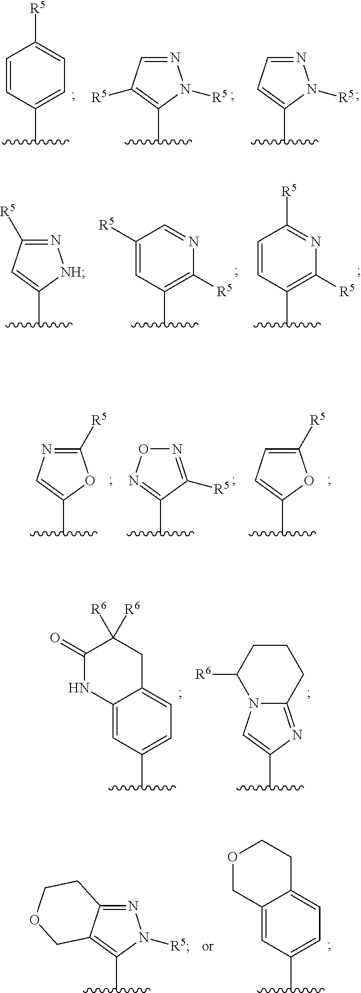

and the remaining variables are as described in the twentieth or twenty-first embodiment.

In a twenty-third embodiment, for the compounds of Formula (III) or (IV), or a pharmaceutically acceptable salt thereof, Het is represented by the following formula:

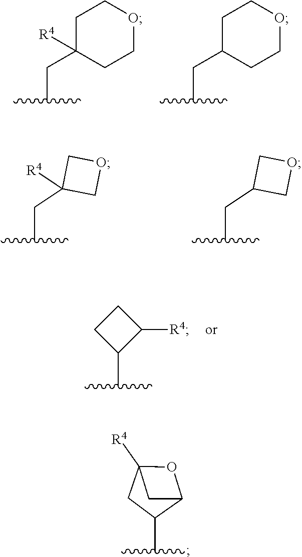

and the remaining variables are as described in the twentieth, twenty-first, or twenty-second embodiment.

In a twenty-fourth embodiment, for the compounds of Formula (III) or (IV), or a pharmaceutically acceptable salt thereof, R⁴, for each occurrence, is independently —CH₃, —OH, or F; and the remaining variables are as described in the twentieth, twenty-first, twenty-second, or twenty-third embodiment.

In a twenty-fifth embodiment, for the compounds of Formula (III) or (IV), or a pharmaceutically acceptable salt thereof, R⁵, for each occurrence, is independently —OCF₃, —OCHF₂, Cl, —CH₂OCH₃, —CH₃, —CF₃, —OCH₃, —CH₂CH₃,

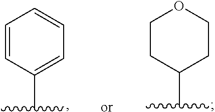

and the remaining variables are as described in the twentieth, twenty-first, twenty-second, twenty-third, or twenty-fourth embodiment.

In a twenty-sixth embodiment, the compound of the present disclosure is represented by Formula V:

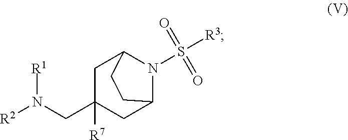

or a pharmaceutically acceptable salt thereof; wherein the variables in Formula (V) are as defined in the first aspect or the first embodiment above.

In a twenty-seventh embodiment, the compound of the present disclosure is represented by Formula (VA) or (VB):

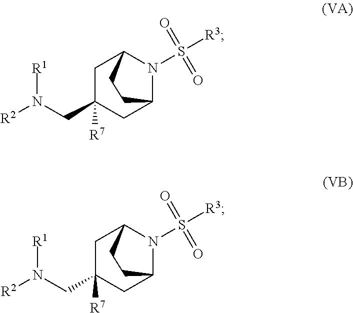

or a pharmaceutically acceptable salt thereof; wherein the variables in Formulas (VA) and (VB) are as defined in the twenty-sixth embodiment above.

In a twenty-eighth embodiment, for the compounds of Formula (V), (VA), or (VB), or a pharmaceutically acceptable salt thereof, R³ is selected from the group consisting of phenyl, pyrazolyl, isoxazoyl, and pyridinyl, each of which is optionally substituted with one to three R⁵; and the remaining variables are as described in the twenty-sixth or twenty-seventh embodiment.

In a twenty-ninth embodiment, for the compounds of Formula (V), (VA), or (VB), or a pharmaceutically acceptable salt thereof, R³ is represented by the following formula:

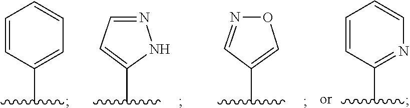

wherein each of the formula depicted above is optionally substituted with one to three R⁵; and the remaining variables are as described in the twenty-sixth, twenty-seventh, or twenty-eighth embodiment.

In a thirtieth embodiment, for the compounds of Formula (V), (VA), or (VB), or a pharmaceutically acceptable salt thereof, R³ is represented by the following formula:

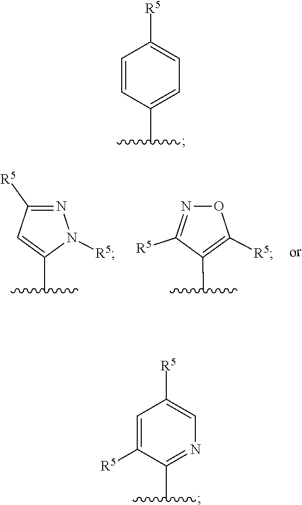

and the remaining variables are as described in the twenty-sixth, twenty-seventh, twenty-eighth, or twenty-ninth embodiment.

In a thirty-first embodiment, for the compounds of Formula (V), (VA), or (VB), or a pharmaceutically acceptable salt thereof, R⁵, for each occurrence, is independently halo, —OR^{5a} or C₁₋₆alkyl; and the remaining variables are as described in the twenty-sixth, twenty-seventh, twenty-eighth, twenty-ninth, or thirtieth embodiment.

In a thirty-second embodiment, for the compounds of Formula (V), (VA), or (VB), or a pharmaceutically acceptable salt thereof, R⁷ is H or OH; and the remaining variables are as described in the twenty-sixth, twenty-seventh, twenty-eighth, twenty-ninth, thirtieth, or thirty-first embodiment.

In a thirty-third embodiment, for the compounds of Formula (V), (VA), or (VB), or a pharmaceutically acceptable salt thereof, R¹ and R² together with the nitrogen atom from which they are attached form a group represented by the following formula:

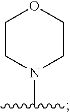

and the remaining variables are as described in the twenty-sixth, twenty-seventh, twenty-eighth, twenty-ninth, thirtieth, thirty-first, or thirty-second embodiment.

In a thirty-fourth embodiment, the compound of the present disclosure is represented by Formula (VI) or (VII):

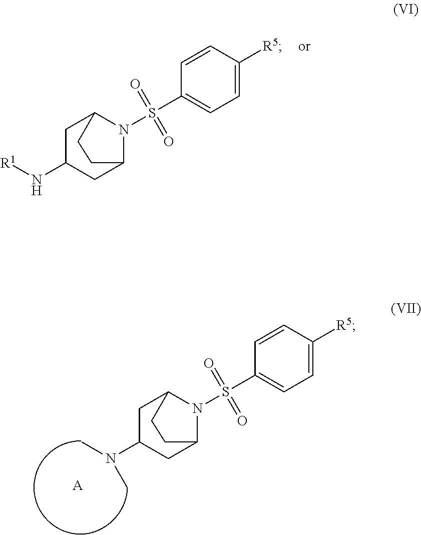

or a pharmaceutically acceptable salt thereof, wherein:
R¹ is C₁₋₆alkyl, C₃₋₆cycloalkyl, Het, —CH₂-C₃₋₄cycloalkyl, or —CH₂-Het, wherein the C₁₋₆alkyl, C₃₋₆cycloalkyl, and —CH₂-C₃₋₄cycloalkyl are each optionally substituted with one or two R⁴;
Het is a 4 to 6 membered monocyclic saturated heterocyclyl containing one oxygen ring atom;
Ring A is a 4 to 6 membered monocyclic saturated heterocyclyl or a 8 or 9 membered bicyclic saturated heterocyclyl, each of which is optionally substituted with one or two R⁴;
R⁴, for each occurrence, is independently selected from-OR^{4a}, —CH₂—OR^{4a}, and C₁₋₃alkyl;
R^{4a} is H or C₁₋₃alkyl;
R⁵ is —OR^{5a};
R^{5a} is C₁₋₃haloalkyl; wherein the remaining variables in Formulas (VI) and (VII) are as defined in the third embodiment.

In a thirty-fifth embodiment, for the compounds of Formula (VI) or (VII), or a pharmaceutically acceptable salt thereof, Het is tetrahydropyranyl or oxetanyl; and the remaining variables are as defined in the thirty-fourth embodiment.

In a thirty-sixth embodiment, for the compounds of Formula (VI) or (VII), or a pharmaceutically acceptable salt thereof, R¹ is:
(i) C₁₋₆alkyl optionally substituted with one or two R⁴;
(ii) represented by the following formula:

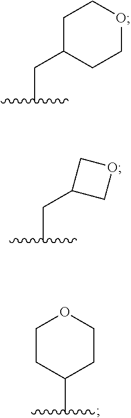

or
(iii) represented by the following formula:

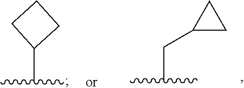

each of which is optionally substituted with one or two R⁴; and the remaining variables are as defined in the thirty-fourth or thirty-fifth embodiment.

In a thirty-seventh embodiment, for the compounds of Formula (VI) or (VII), or a pharmaceutically acceptable salt thereof, R¹ is —CH(CH₃)CH₂CH₂OCH₃ or CH₂C(CH₃)₂OH, or R¹ is represented by the following formula

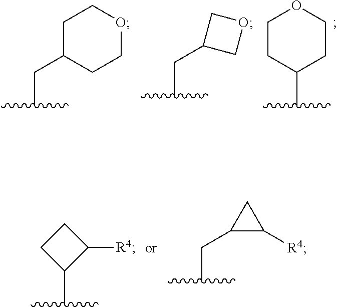

and the remaining variables are as defined in the thirty-sixth embodiment.

In a thirty-eighth embodiment, for the compounds of Formula (VI) or (VII), or a pharmaceutically acceptable salt thereof, Ring A is

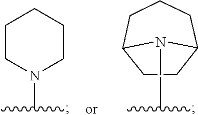

each of which is optionally substituted with one to two R⁴; and the remaining variables are as defined in the thirty-fourth embodiment.

In a thirty-ninth embodiment, for the compounds of Formula (VI) or (VII), or a pharmaceutically acceptable salt thereof, Ring A is

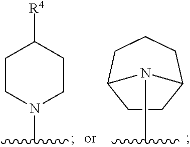

and the remaining variables are as defined in the thirty-eighth embodiment.

In a fortieth embodiment, for the compounds of Formula (VI) or (VII), or a pharmaceutically acceptable salt thereof, each R⁴ is independently —OH, —OCH₃, —CH₂OH, or —CH₃; and the remaining variables are as described in the thirty-fourth, thirty-fifth, thirty-sixth, thirty-seventh, thirty-eighth, or thirty-ninth embodiment.

In a forty-first embodiment, for the compounds of Formula (VI) or (VII), or a pharmaceutically acceptable salt thereof, R⁵ is —OCHF₂; and the remaining variables are as described in the thirty-fourth, thirty-fifth, thirty-sixth, thirty-seventh, thirty-eighth, thirty-ninth, or fortieth embodiment.

In a forty-second embodiment, the present disclosure provides a compound described herein (e.g., a compound of any one of Examples 1 to 77), or a pharmaceutically acceptable salt thereof.

In a forty-third embodiment, the present disclosure provides a compound selected from the group consisting of:
3-Methyl-4-(((1R,3r,5S)-3-(4-methylpiperidin-1-yl)-8-azabicyclo[3.2.1]octan-8-yl)sulfonyl)-1,2,5-oxadiazole;
5-(((1R,3r,5S)-3-(4-Methylpiperidin-1-yl)-8-azabicyclo[3.2.1]octan-8-yl)sulfonyl)-2-(tetrahydro-2H-pyran-4-yl) oxazole;
(1R,3s,5S)-8-((2-Methoxy-6-methylpyridin-3-yl)sulfonyl)-3-(4-methylpiperidin-1-yl)-8-azabicyclo[3.2.1]octane;
(1R,3s,5S)-8-((4-Chloro-1-(methoxymethyl)-1H-pyrazol-5-yl)sulfonyl)-3-(4-methylpiperidin-1-yl)-8-azabicyclo[3.2.1]octane;
3,3-Dimethyl-6-(((1R,3s,5S)-3-(4-methylpiperidin-1-yl)-8-azabicyclo[3.2.1]octan-8-yl)sulfonyl)-3,4-dihydroquinolin-2 (1H)-one;
5-(((1R,3s,5S)-3-(4-Methylpiperidin-1-yl)-8-azabicyclo[3.2.1]octan-8-yl)sulfonyl)-2-(tetrahydro-2H-pyran-4-yl) oxazole;
(1R,3s,5S)-8-(Isochroman-7-ylsulfonyl)-3-(4-methylpiperidin-1-yl)-8-azabicyclo[3.2.1]octane;
(1R,3s,5S)-8-((5-(Methoxymethyl) furan-2-yl)sulfonyl)-3-(4-methylpiperidin-1-yl)-8-azabicyclo[3.2.1]octane;
2-Methyl-3-(((1R,3s,5S)-3-(4-methylpiperidin-1-yl)-8-azabicyclo[3.2.1]octan-8-yl)sulfonyl)-2,4,6,7-tetrahydropyrano[4,3-c]pyrazole;
(1R,3s,5S)-3-(4-Methylpiperidin-1-yl)-8-((3-phenyl-1H-pyrazol-5-yl)sulfonyl)-8-azabicyclo[3.2.1]octane;
5-Methyl-2-(((1R,3s,5S)-3-(4-methylpiperidin-1-yl)-8-azabicyclo[3.2.1]octan-8-yl)sulfonyl)-5,6,7,8-tetrahydroimidazo[1,2-a]pyridine;
(1R,3s,5S)-8-((1,4-Dimethyl-1H-pyrazol-5-yl)sulfonyl)-3-(4-methylpiperidin-1-yl)-8-azabicyclo[3.2.1]octane;
(1R,3s,5S)-8-((1-Ethyl-1H-pyrazol-5-yl)sulfonyl)-3-(4-methylpiperidin-1-yl)-8-azabicyclo[3.2.1]octane;
2-((8-((4-(Difluoromethoxy)phenyl)sulfonyl)-8-azabicyclo[3.2.1]octan-3-yl)amino)cyclobutan-1-ol;
8-((4-(Difluoromethoxy)phenyl)sulfonyl)-N-((3-methyloxetan-3-yl)methyl)-8-azabicyclo[3.2.1]octan-3-amine;
9-(8-((4-(Difluoromethoxy)phenyl)sulfonyl)-8-azabicyclo[3.2.1]octan-3-yl)-3,3-difluoro-9-azabicyclo[3.3.1]nonane;
4-((((1R,3r,5S)-8-((4-(Difluoromethoxy)phenyl)sulfonyl)-8-azabicyclo[3.2.1]octan-3-yl)amino)methyl)tetrahydro-2H-pyran-4-ol;
4-((((1R,3s,5S)-8-((4-(Difluoromethoxy)phenyl)sulfonyl)-8-azabicyclo[3.2.1]octan-3-yl)amino)methyl)tetrahydro-2H-pyran-4-ol;
8-((4-(Difluoromethoxy)phenyl)sulfonyl)-N-((tetrahydro-2H-pyran-4-yl)methyl)-8-azabicyclo[3.2.1]octan-3-amine;
(1R,3r,5S)-8-((4-(Difluoromethoxy)phenyl)sulfonyl)-3-(4-methylpiperidin-1-yl)-8-azabicyclo[3.2.1]octane;
(1R,3s,5S)-8-((4-(Difluoromethoxy)phenyl)sulfonyl)-3-(4-methylpiperidin-1-yl)-8-azabicyclo[3.2.1]octane;
6-((1R,3s,5S)-8-((2-Methyl-6-(trifluoromethyl)pyridin-3-yl)sulfonyl)-8-azabicyclo[3.2.1]octan-3-yl)-2-oxa-6-azaspiro[3.3]heptane;
6-((1R,3r,5S)-8-((2-Methyl-6-(trifluoromethyl)pyridin-3-yl)sulfonyl)-8-azabicyclo[3.2.1]octan-3-yl)-2-oxa-6-azaspiro[3.3]heptane;
(1R,3s,5S)-8-((2-Methyl-6-(trifluoromethyl)pyridin-3-yl)sulfonyl)-N-((tetrahydro-2H-pyran-4-yl)methyl)-8-azabicyclo[3.2.1]octan-3-amine;
(1R,3r,5S)-8-((2-Methyl-6-(trifluoromethyl)pyridin-3-yl)sulfonyl)-N-((tetrahydro-2H-pyran-4-yl)methyl)-8-azabicyclo[3.2.1]octan-3-amine;
(1R,3s,5S)-8-((2-Methyl-6-(trifluoromethyl)pyridin-3-yl)sulfonyl)-N-((3-methyloxetan-3-yl)methyl)-8-azabicyclo[3.2.1]octan-3-amine;
(1R,3r,5S)-8-((2-Methyl-6-(trifluoromethyl)pyridin-3-yl)sulfonyl)-N-((3-methyloxetan-3-yl)methyl)-8-azabicyclo[3.2.1]octan-3-amine;
(1R,3s,5S)-8-((4-(Difluoromethoxy)phenyl)sulfonyl)-N-((3-methyloxetan-3-yl)methyl)-8-azabicyclo[3.2.1]octan-3-amine;
(1R,3s,5S)-8-((4-(Difluoromethoxy)phenyl)sulfonyl)-N-((tetrahydro-2H-pyran-4-yl)methyl)-8-azabicyclo[3.2.1]octan-3-amine;
8-((4-(Difluoromethoxy)phenyl)sulfonyl)-N-(1-methyl-2-oxabicyclo[2.1.1]hexan-4-yl)-8-azabicyclo[3.2.1]octan-3-amine;
(1R,3s,5S)-8-((4-(Difluoromethoxy)phenyl)sulfonyl)-N-(oxetan-3-ylmethyl)-8-azabicyclo[3.2.1]octan-3-amine;
(1R,3r,5S)-8-((4-(Difluoromethoxy)phenyl)sulfonyl)-N-((tetrahydro-2H-pyran-4-yl)methyl)-8-azabicyclo[3.2.1]octan-3-amine;
(1R,3r,5S)-8-((4-(Difluoromethoxy)phenyl)sulfonyl)-3-(morpholinomethyl)-8-azabicyclo[3.2.1]octan-3-ol;
(1R,3s,5S)-8-((4-(Difluoromethoxy)phenyl)sulfonyl)-3-(morpholinomethyl)-8-azabicyclo[3.2.1]octan-3-ol;
4-(((1R,3s,5S)-8-((4-(Difluoromethoxy)-2,6-difluorophenyl)sulfonyl)-8-azabicyclo[3.2.1]octan-3-yl)methyl)morpholine;
4-(((1R,3s,5S)-8-((1,3-Dimethyl-1H-pyrazol-5-yl)sulfonyl)-8-azabicyclo[3.2.1]octan-3-yl)methyl)morpholine;
4-(((1R,3r,5S)-8-((1,3-Dimethyl-1H-pyrazol-5-yl)sulfonyl)-8-azabicyclo[3.2.1]octan-3-yl)methyl)morpholine;
4-(((1R,3s,5S)-8-((3,5-Dimethylisoxazol-4-yl)sulfonyl)-8-azabicyclo[3.2.1]octan-3-yl)methyl)morpholine;
4-(((1R,3s,5S)-8-((4,6-Dimethylpyridin-3-yl)sulfonyl)-8-azabicyclo[3.2.1]octan-3-yl)methyl)morpholine;
(1R,3r,5S)-3-(4-Methylpiperidin-1-yl)-8-((1-(4-methylpyridin-2-yl)-1H-pyrazol-4-yl)sulfonyl)-8-azabicyclo[3.2.1]octane;
(1R,3s,5S)-3-(4-Methylpiperidin-1-yl)-8-((1-(4-methylpyridin-2-yl)-1H-pyrazol-4-yl)sulfonyl)-8-azabicyclo[3.2.1]octane;
4-(((1R,3s,5S)-3-(4-Methylpiperidin-1-yl)-8-azabicyclo[3.2.1]octan-8-yl)sulfonyl)-2-(trifluoromethyl) thiazole;
(1R,3s,5S)-8-((2,6-Difluoro-4-methoxyphenyl)sulfonyl)-3-(4-methylpiperidin-1-yl)-8-azabicyclo[3.2.1]octane;
(1R,3s,5S)-3-(4-Methylpiperidin-1-yl)-8-((1-phenyl-1H-1,2,3-triazol-4-yl)sulfonyl)-8-azabicyclo[3.2.1]octane;
1,3-Dimethyl-5-(((1R,3s,5S)-3-(4-methylpiperidin-1-yl)-8-azabicyclo[3.2.1]octan-8-yl)sulfonyl)-1H-pyrazolo[3,4-b]pyridine;
(1R,3s,5S)-8-((3,5-Dimethylpyridin-2-yl)sulfonyl)-3-(4-methylpiperidin-1-yl)-8-azabicyclo[3.2.1]octane;
(1R,3s,5S)-8-((2,6-Dimethylpyridin-3-yl)sulfonyl)-3-(4-methylpiperidin-1-yl)-8-azabicyclo[3.2.1]octane;
1-((8-((4-(Difluoromethoxy)phenyl)sulfonyl)-8-azabicyclo[3.2.1]octan-3-yl)amino) propan-2-ol;
1-(8-((4-(Difluoromethoxy)phenyl)sulfonyl)-8-azabicyclo[3.2.1]octan-3-yl)-3-methylpyrrolidin-3-ol;
8-((4-(Difluoromethoxy)phenyl)sulfonyl)-3-(3-methoxypyrrolidin-1-yl)-8-azabicyclo[3.2.1]octane;
6-(8-((4-(Difluoromethoxy)phenyl)sulfonyl)-8-azabicyclo[3.2.1]octan-3-yl)-2-oxa-6-azaspiro[3.4]octane;
8-((4-(Difluoromethoxy)phenyl)sulfonyl)-N-(4-methoxybutan-2-yl)-8-azabicyclo[3.2.1]octan-3-amine;
8-((4-(Difluoromethoxy)phenyl)sulfonyl)-8,8′-diaza-3,8′-bi(bicyclo[3.2.1]octane);
8-((4-(Difluoromethoxy)phenyl)sulfonyl)-3-(3-(2-methoxyethyl) azetidin-1-yl)-8-azabicyclo[3.2.1]octane;
8-((4-(Difluoromethoxy)phenyl)sulfonyl)-N-(3-methoxybutan-2-yl)-8-azabicyclo[3.2.1]octan-3-amine;
rac-((1R,2S)-2-(((8-((4-(Difluoromethoxy)phenyl)sulfonyl)-8-azabicyclo[3.2.1]octan-3-yl)amino)methyl)cyclopropyl)methanol;
3-(8-((4-(Difluoromethoxy)phenyl)sulfonyl)-8-azabicyclo[3.2.1]octan-3-yl)-3-azabicyclo[3.1.1]heptane;
8′-((4-(Difluoromethoxy)phenyl)sulfonyl)-3,8′-diaza-3,3′-bi(bicyclo[3.2.1]octane); rac-4-(((1R,3r,5S)-8-((4-(Difluoromethoxy)phenyl)sulfonyl)-8-azabicyclo[3.2.1]octan-3-yl)amino)butan-2-ol;
rac-4-(((1R,3s,5S)-8-((4-(Difluoromethoxy)phenyl)sulfonyl)-8-azabicyclo[3.2.1]octan-3-yl)amino)butan-2-ol;
(1R,3r)-3-((((1R,3s,5S)-8-((4-(Difluoromethoxy)phenyl)sulfonyl)-8-azabicyclo[3.2.1]octan-3-yl)amino)methyl)cyclobutan-1-ol; (1R,3r)-3-((((1R,3r,5S)-8-((4-(Difluoromethoxy)phenyl)sulfonyl)-8-azabicyclo[3.2.1]octan-3 yl)amino)methyl)cyclobutan-1-ol;
(1S,3s)-3-((((1R,3s,5S)-8-((4-(Difluoromethoxy)phenyl)sulfonyl)-8-azabicyclo[3.2.1]octan-3-yl)amino)methyl)cyclobutan-1-ol;
(1S,3s)-3-((((1R,3r,5S)-8-((4-(Difluoromethoxy)phenyl)sulfonyl)-8-azabicyclo[3.2.1]octan-3-yl)amino)methyl)cyclobutan-1-ol;
(1R,3r,5S)-8-((4-(Difluoromethoxy)phenyl)sulfonyl)-N-methyl-N-(tetrahydro-2H-pyran-4-yl)-8-azabicyclo[3.2.1]octan-3-amine;
(1R,3s,5S)-8-((4-(Difluoromethoxy)phenyl)sulfonyl)-N-methyl-N-(tetrahydro-2H-pyran-4-yl)-8-azabicyclo[3.2.1]octan-3-amine;
4-(((1R,3s,5S)-8-((4-(Difluoromethoxy)phenyl)sulfonyl)-8-azabicyclo[3.2.1]octan-3-yl)methyl)morpholine;
(1R,3s,5S)-8-((2-Methyl-6-(trifluoromethyl)pyridin-3-yl)sulfonyl)-N-(2-oxaspiro[3.3]heptan-6-yl)-8-azabicyclo[3.2.1]octan-3-amine
(1R,3r,5S)-8-((2-Methyl-6-(trifluoromethyl)pyridin-3-yl)sulfonyl)-N-(2-oxaspiro[3.3]heptan-6-yl)-8-azabicyclo[3.2.1]octan-3-amine;
8-((4-(Difluoromethoxy)phenyl)sulfonyl)-N—((R)-tetrahydrofuran-3-yl)-8-azabicyclo[3.2.1]octan-3-amine;
(1R,3r,5S)-8-((4-(Difluoromethoxy)phenyl)sulfonyl)-N-((3-fluorooxetan-3-yl)methyl)-8-azabicyclo[3.2.1]octan-3-amine;
(1R,3r,5S)-8-((4-(Difluoromethoxy)phenyl)sulfonyl)-N-(oxetan-3-ylmethyl)-8-azabicyclo[3.2.1]octan-3-amine;
8-((4-(Difluoromethoxy)phenyl)sulfonyl)-N-(tetrahydro-2H-pyran-4-yl)-8-azabicyclo[3.2.1]octan-3-amine;
8-((4-(Difluoromethoxy)phenyl)sulfonyl)-N-(oxetan-3-yl)-8-azabicyclo[3.2.1]octan-3-amine;
(1R,3s,5S)-8-((2-Methyl-6-(trifluoromethyl)pyridin-3-yl)sulfonyl)-N-(tetrahydro-2H-pyran-4-yl)-8-azabicyclo[3.2.1]octan-3-amine;
1-(((1R,3s,5S)-8-((4-(Difluoromethoxy)phenyl)sulfonyl)-8-azabicyclo[3.2.1]octan-3-yl)amino)-2-methylpropan-2-ol; and
1-(((1R,3r,5S)-8-((4-(Difluoromethoxy)phenyl)sulfonyl)-8-azabicyclo[3.2.1]octan-3-yl)amino)-2-methylpropan-2-ol;
   or a pharmaceutically acceptable salt thereof.

In a forty-fourth embodiment, the present disclosure provides a pharmaceutical composition comprising a compound according to any one of the preceding embodiments, or a pharmaceutically acceptable salt thereof.

In a forty-fifth embodiment, the present disclosure provides a method of treating a disease or disorder mediated by EBP comprising administering to a subject an effective amount of a compound according to any one of embodiments one to forty-four, or a pharmaceutically acceptable salt thereof, or the pharmaceutical composition of the forty-fourth embodiment.

In a forty-sixth embodiment, the present disclosure provides a compound according to any one of embodiments one to forty-four, for use in the treatment of a disease or disorder mediated by EBP.

In a forty-seventh embodiment, the present disclosure provides the use of a compound according to any one of embodiments one to forty-four in the manufacture of a medicament for the treatment of a disease or disorder mediated by EBP.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca. Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the invention include those wherein the solvent of crystallization may be isotopically substituted, e.g. D₂O, de-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional R—S system (e.g., (1S,2S)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)— or (S)—.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Methods of Use

The compounds disclosed herein have EBP inhibitory activity. As used herein, “EBP inhibitory activity” refers to the ability of a compound or composition to induce a detectable decrease in EBP activity in vivo or in vitro (e.g., at least 10% decrease in EBP activity as measured by a given assay such as the bioassay described in the examples and known in the art).

In certain embodiments, the present disclosure provides a method of treating a disease or disorder responsive to inhibition of EBP activity (referred herein as “EBP mediated disease or disorder” or “disease or disorder mediated by EBP”) in a subject in need of the treatment. The method comprises administering to the subject a compound described herein (e.g., a compound described in any one of the first to forty-third embodiments) or a pharmaceutically acceptable salt thereof or a pharmaceutical composition thereof.

In certain embodiments, the present disclosure provides the use of a compound described herein (e.g., a compound described in any one of the first to forty-third embodiments) or a pharmaceutically acceptable salt thereof or a pharmaceutical composition comprising a compound described herein or a pharmaceutically acceptable salt thereof for the manufacture of a medicament for the treatment of a EBP mediated disorder or disease in a subject in need of the treatment.

In certain embodiments, the present disclosure provides a compound described herein (e.g., a compound described in any one of the first to forty-third embodiments) or a pharmaceutically acceptable salt thereof or a pharmaceutical composition comprising a compound described herein or a pharmaceutically acceptable salt thereof for use in the treatment of a EBP mediated disorder or disease in a subject in need of the treatment.

In certain embodiments, the EBP mediated disorder is colorectal cancer.

In certain embodiments, the present disclosure provides a method of treating an autoimmune disease in a subject in need of the treatment. The method comprises administering to the subject a compound described herein (e.g., a compound described in any one of the first to forty-third embodiments) or a pharmaceutically acceptable salt thereof or a pharmaceutical composition thereof.

In certain embodiments, the present disclosure provides the use of a compound described herein (e.g., a compound described in any one of the first to forty-third embodiments) or a pharmaceutically acceptable salt thereof or a pharmaceutical composition comprising a compound described herein or a pharmaceutically acceptable salt thereof for the manufacture of a medicament for the treatment of an autoimmune disease in a subject in need of the treatment.

In certain embodiments, the present disclosure provides a compound described herein (e.g., a compound described in any one of the first to forty-third embodiments) or a pharmaceutically acceptable salt thereof or a pharmaceutical composition comprising a compound described herein or a pharmaceutically acceptable salt thereof for use in the treatment of an autoimmune disease in a subject in need of the treatment.

In certain embodiments, the autoimmune disease is multiple sclerosis (MS). The compounds of the present disclosure can be used for treating all stages of MS, including relapsing multiple sclerosis (or relapsing form(s) of multiple sclerosis), relapsing-remitting multiple sclerosis, primary progress multiple sclerosis, secondary progressive multiple sclerosis and clinically isolated syndrome (hereinafter “CIS”).

Relapsing multiple sclerosis (or relapsing form(s) of multiple sclerosis) includes clinically isolated syndrome, relapsing-remitting multiple sclerosis and active secondary progressive multiple sclerosis.

Relapsing-remitting multiple sclerosis is a stage of MS characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with multiple sclerosis.

Secondary progressive multiple sclerosis occurs in around 65% of those with initial relapsing-remitting multiple sclerosis, who eventually have progressive neurologic decline between acute attacks without any definite periods of remission. Occasional relapses and minor remissions may appear. The most common length of time between disease onset and conversion from relapsing-remitting to secondary progressive multiple sclerosis is 19 years.

Primary progressive multiple sclerosis is characterized by the same symptoms of secondary progressive multiple sclerosis, i.e., progressive neurologic decline between acute attacks without any definite periods of remission, without the prior relapsing-remitting stage.

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS. When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

In certain embodiments, the present disclosure provides a method of promoting myelination in a subject with a myelin-related disease or disorder in a subject in need of the treatment. The method comprises administering to the subject a compound described herein (e.g., a compound described in any one of the first to forty-third embodiments) or a pharmaceutically acceptable salt thereof or a pharmaceutical composition thereof.

In certain embodiments, the present disclosure provides the use of a compound described herein (e.g., a compound described in any one of the first to forty-third embodiments) or a pharmaceutically acceptable salt thereof or a pharmaceutical composition comprising a compound described herein or a pharmaceutically acceptable salt thereof for the manufacture of a medicament for promoting myelination in a subject with a myelin-related disease or disorder in a subject in need of the treatment.

In certain embodiments, the present disclosure provides a compound described herein (e.g., a compound described in any one of the first to forty-third embodiments) or a pharmaceutically acceptable salt thereof or a pharmaceutical composition comprising a compound described herein or a pharmaceutically acceptable salt thereof for use in promoting myelination in a subject with a myelin-related disease or disorder in a subject in need of the treatment.

In certain embodiments, the myelin-related disease or disorder is selected from multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), acute disseminated encephalomyelitis (ADEM), central pontine myelolysis (CPM), adrenoleukodystrophy, Alexander's disease, Pelizacus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, autism, metachromatic leukodystrophy, trigeminal neuralgia, acute disseminated encephalitis, chronic inflammatory demyelinating polyneuropathy, Guillian-Barre syndrome, Charcot-Marie-Tooth disease, Bell's palsy and radiation-induced demyelination, for example, neuromyelitis optica (NMO), optic neuritis, pediatric leukodystrophies, neonatal white matter injury, age-related dementia, and schizophrenia.

In certain embodiments, the present disclosure provides a method of treating cancer in a subject in need of the treatment. The method comprises administering to the subject a compound described herein (e.g., a compound described in any one of the first to forty-third embodiments) or a pharmaceutically acceptable salt thereof or a pharmaceutical composition thereof.

In certain embodiments, the present disclosure provides the use of a compound described herein (e.g., a compound described in any one of the first to forty-third embodiments) or a pharmaceutically acceptable salt thereof or a pharmaceutical composition comprising a compound described herein or a pharmaceutically acceptable salt thereof for the manufacture of a medicament for the treatment of cancer in a subject in need of the treatment.

In certain embodiments, the present disclosure provides a compound described herein (e.g., a compound described in any one of the first to forty-third embodiments) or a pharmaceutically acceptable salt thereof or a pharmaceutical composition comprising a compound described herein or a pharmaceutically acceptable salt thereof for use in treating cancer in a subject in need of the treatment.

In certain embodiments, the cancer is colorectal cancer.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 μg and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g., intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose of less than or equal to about 100 mg/kg, 75 mg/kg, 50 mg/kg. 25 mg/kg, 10 mg/kg, 7.5 mg/kg, 5.0 mg/kg. 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”. “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein, the term “cancer” has the meaning normally accepted in the art. The term can broadly refer to abnormal cell growth.

As used herein, the term “autoimmune disease” has the meaning normally accepted the art. The term can broadly refer to a disease where the host's immune system targets or attacks normal or healthy tissue of the host.

As used herein, the term “myelination” has the meaning normally accepted in the art. The term can broadly mean the process by which myelin is produced.

As used herein, the term “myelin-related disease or disorder”, “demyelinating disorder”, or “demyelation disorder” has the meaning normally accepted in the art. These terms can broadly refer to diseases or disorders which involve damage to myelin.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄ allkyl group as defined herein, wherein at least of the hydrogen atoms is replaced by an C₁₋₄ alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄ alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyrazolyl, imidazolyl, oxazolyl, pyridinyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indoyl.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 6 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form. In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

### EXEMPLIFICATION

### Abbreviations

PE=petroleum ether
EtOAc=EA=ethyl acetate
ESI=electrospray ionisation
MeOH=methanol
EtOH=ethanol
DCE=1,2-dichloroethane
DCM=dichloromethane
CHCl₃=chloroform
HCl=hydrochloric acid
H₂O=water
IPA=isopropyl alcohol
LCMS=liquid chromatography mass spectrometry
HFIP=hexafluoro-2-propanol
HPLC=high pressure liquid chromatography
THF=tetrahydrofuran
MeCN=ACN=acetonitrile
MgSO₄=magnesium sultate
DMSO=dimetylsulfoxide
AcOH=acetic acid
TFA=trifluoroacetic acid
DIPEA=diisopropylethyl amine
N₂=Nitrogen
NH₄HCO₃=Ammonium Bicarbonate
t-BuOH=tert-butanol
NH₄Cl=ammonium chloride
NaH=sodium hydride
Na₂SO₄=sodium sulfate
K₂CO₃=potassium carbonate
NaHCO₃=sodium bicarbonate
NaBH(OAc)₃=STAB=sodium triacetoxyborohydride
SiO₂=silicon dioxide or silica
PDA=Photo Diode Array Detection

### GENERAL METHODS

### LCMS Instrumentation Specifications:

Agilent Technologies 1200 Series LC/MSD system: DAD\ELSD Alltech 3300 and Agilent LC\MSD G6130A, G6120B mass-spectrometer.
Agilent Technologies 1260 Infinity LC/MSD system: DAD\ELSD Alltech 3300 and Agilent LC\MSD G6120B mass-spectrometer.
Agilent Technologies 1260 Infinity II LC/MSD system: DAD\ELSD G7102A 1290 Infinity II and Agilent LC\MSD G6120B mass-spectrometer.
Agilent 1260 Series LC/MSD system: DADELSD and Agilent LC\MSD (G6120B) mass-spectrometer.
UHPLC Agilent 1290 Series LC/MSD system: DADIELSD and Agilent LC\MSD (G6125B) mass-spectrometer.

### HPLC Analytical Method Specifications:

Column: Agilent Poroshell 120 SB-C18 4.6×30 mm 2.7 μm, with UHPLC Guard Infinity Lab Poroshell 120 SB-C18 4.6×5 mm 2.7 μm
Column Temperature, 60 C
Injection volume 0.5 μL
Modifier: Formic acid 0.1% (v/v) conc.
Method: 99% water/1% MeCN (initial conditions), HOLD initial conditions for 0.1 min, linear gradient to 0% water/100% MeCN at 1.5 min, HOLD 0% water/100% MeCN to 1.73 min, linear gradient to 99% water/1% MeCN at 1.74 min. Flow rate, 3.0 mL/min.
UV scan: 207-223 nm, 246-262 nm, 272-288 nm

### QC Analysis LC/MS Method Conditions:

### Ammonium Hydroxide (Basic pH) Conditions

MS mode: MS: ESI+ scan range 165-650 daltons
PDA: 200-400 nm scan range
Column: Waters ACQUITY UPLC BEH C18 2.1×50 mm, 1.7 μm; Part No. 186002350
Modifier: Ammonium hydroxide 0.2% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### Trifluoroacetic Acid (Acidic pH) Conditions

MS mode: MS: ESI+ scan range 165-650 daltons
PDA: 200-400 nm scan range
Column: Waters ACQUITY UPLC BEH C18 2.1×50 mm, 1.7 μm; Part No. 186002350
Modifier: Trifluoroacetic acid 0.1% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### General Prep HPLC Conditions:

### Ammonium Hydroxide (Basic pH) Conditions

Flow rate: 30 mL/min
MS mode: MS: ESI+ scan range 165-650 daltons
PDA: 200-400 nm scan range
Column: Waters XSELECT CSH C18 PREP 19×100 mm, 5 μm; Part No. 186005421
Modifier: 0.2% Ammonium hydroxide (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate: 50 mL/min
MS mode: MS: ESI+ scan range 165-650 daltons
PDA: 200-400 nm scan range
Column: Waters XSELECT CSH C18 PREP 30×100 mm, 5 μm; Part No. 186005425
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS: ESI+ scan range 165-650 daltons
PDA: 200-400 nm scan range
Column: Waters XSELECT CSH C18 PREP 30×50 mm, 5 μm; Part No. 186005423
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### Trifluoroacetic Acid (Acidic pH) Conditions

Flow rate, 30 mL/min
MS mode: MS: ESI+ scan range 165-650 daltons
PDA: 200-400 nm scan range
Column: Waters Sunfire OBD C18 PREP 19×100 mm, 5 μm; Part No. 186002567
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 50 mL/min
MS mode: MS: ESI+ scan range 165-650 daltons
PDA: 200-400 nm scan range
Column: Waters Sunfire OBD C18 PREP 30×100 mm, 5 μm; Part No. 186002572
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS: ESI+ scan range 165-650 daltons
PDA: 200-400 nm scan range
Column: Waters Sunfire OBD C18 PREP 30×50 mm, 5 μm; Part No. 186002570
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### PREPARATION OF INTERMEDIATES

### Intermediate 1: (1R,3r,5S)-3-(4-Methylpiperidin-1-yl)-8-azabicyclo[3.2.1]octane

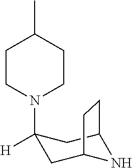

### 1. Synthesis of tert-butyl (1R,3r,5S)-3-(4-methylpiperidin-1-yl)-8-azabicyclo[3.2.1]octane-8-carboxylate

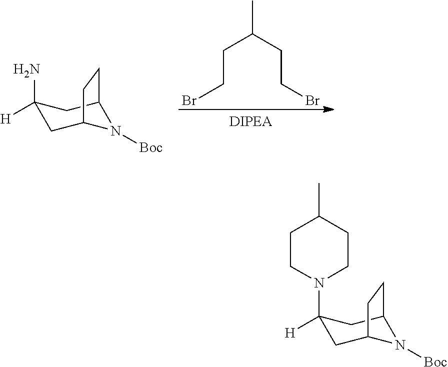

A mixture of tert-butyl (1R,3r,5S)-3-amino-8-azabicyclo[3.2.1]octane-8-carboxylate (15 g, 66.3 mmol), 1,5-dibromo-3-methylpentane (17.79 g, 72.9 mmol) and DIPEA (25.4 mL, 145.8 mmol,) in IPA (250 mL) was heated under reflux for 72 h. The solvent was removed in vacuo and the residue diluted with water (100 mL) and extracted with EtOAc (3×50 mL). The combined organics were dried (Na₂SO₄), filtered, and evaporated to dryness to give tert-butyl (1R,3r,5S)-3-(4-methylpiperidin-1-yl)-8-azabicyclo[3.2.1]octane-8-carboxylate as a yellow oil (19 g, crude; purity 90%) which was used without further purification in the next step. LCMS m/z=309.2 [M+H]⁺.

### 2. Synthesis of (1R,3r,5S)-3-(4-methylpiperidin-1-yl)-8-azabicyclo[3.2.1]octane

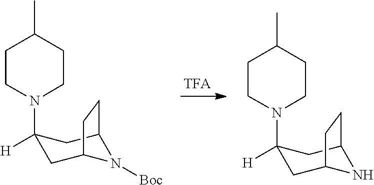 [SMILES: CC1CCN(C2CC3CCC(C2)N3C(=O)OC(C)(C)C)CC1 ; CC1CCN(C2CC3CCC(C2)N3)CC1]

To a solution of tert-butyl (1R,3r,5S)-3-(4-methylpiperidin-1-yl)-8-azabicyclo[3.2.1]octane-8-carboxylate (18.8 g, 61.0 mmol) in DCM (100 mL) was added TFA (14 mL, 183 mmol) and the resulting solution stirred at room temperature for 48 h. The reaction mixture was evaporated to dryness in vacuo and the residue diluted with sat. aq. NaHCO₃ and extracted with EtOAc (3×50 mL). The combined organics were washed with water (50 mL), brine (50 mL), dried (Na₂SO₄), filtered, and evaporated to dryness in vacuo to afford (1R,3r,5S)-3-(4-methylpiperidin-1-yl)-8-azabicyclo[3.2.1]octane as a yellow solid (11.2 g, 88%) which was used without further purification. LCMS m/z=209.2 [M+H]⁺.

### Intermediate 2: (1R,3s,5S)-3-(4-Methylpiperidin-1-yl)-8-azabicyclo[3.2.1]octane

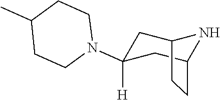

### 1. Synthesis of tert-butyl (1R,3s,5S)-3-(4-methylpiperidin-1-yl)-8-azabicyclo[3.2.1]octane-8-carboxylate

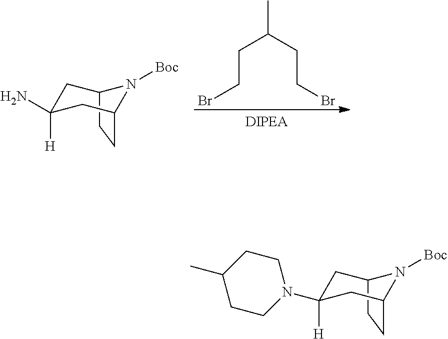

DIPEA (340 μL, 1.9 mmol) was added to a solution of tert-butyl (1R,3s,5S)-3-amino-8-azabicyclo[3.2.1]octane-8-carboxylate (200 mg, 0.9 mmol) and 1,5-dibromo-3-methyl-pentane (237 mg, 1.0 mmol) in IPA (10 mL) and the reaction mixture was heated under reflux for 72 h. The reaction mixture was evaporated to dryness in vacuo. The residue was diluted with water (10 mL) and extracted with EtOAc (3×10 mL). The combined organics were dried (Na₂SO₄), filtered, and evaporated to dryness to afford tert-butyl (1R,3s,5S)-3-(4-methylpiperidin-1-yl)-8-azabicyclo[3.2.1]octane-8-carboxylate as a yellow oil (200 mg, crude) which was used without further purification. LCMS m/z=309.2 [M+H]⁺.

### 2. Synthesis of (1R,3s,5S)-3-(4-methylpiperidin-1-yl)-8-azabicyclo[3.2.1]octane

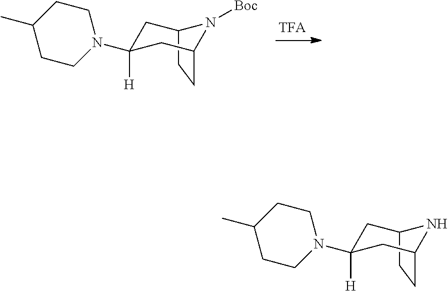 [SMILES: CC1CCN(C2CC3CCC(C2)N3C(=O)OC(C)(C)C)CC1 ; CC1CCN(C2CC3CCC(C2)N3)CC1]

TFA (13.8 mL, 180 mmol) was added to a solution of tert-butyl (1R,3s,5S)-3-(4-methylpiperidin-1-yl)-8-azabicyclo[3.2.1]octane-8-carboxylate (18.5 g, 60 mmol) in DCM (100 mL) and the resulting solution stirred at room temperature for 48 h. The reaction mixture was concentrated under reduced pressure and the residue was taken up in sat. aq. NaHCO₃ and extracted with EtOAc (3×50 mL). The combined organics were washed with water (50 mL), brine (50 mL), dried (Na₂SO₄), filtered, and evaporated to dryness in vacuo. The resulting yellow oil was crystallized from DCM/Hexanes (100/300 mL) and the solids collected by cold filtration and air-dried to afford (1R,3s,5S)-3-(4-methylpiperidin-1-yl)-8-azabicyclo[3.2.1]octane as a yellow solid (6.3 g, 50%). LCMS m/z=209.2 [M+H]⁺.

### Intermediate 3: (1R,3r,5S)-8-((4-(Difluoromethoxy)phenyl)sulfonyl)-8-azabicyclo[3.2.1]octan-3-amine

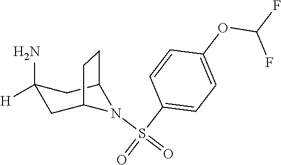 [SMILES: NC1CC2CCC(C1)N2S(=O)(=O)c1ccc(OC(F)F)cc1]

### 1. Synthesis of tert-butyl ((1R,3r,5S)-8-((4-(difluoromethoxy)phenyl)sulfonyl)-8-azabicyclo[3.2.1]octan-3-yl)carbamate

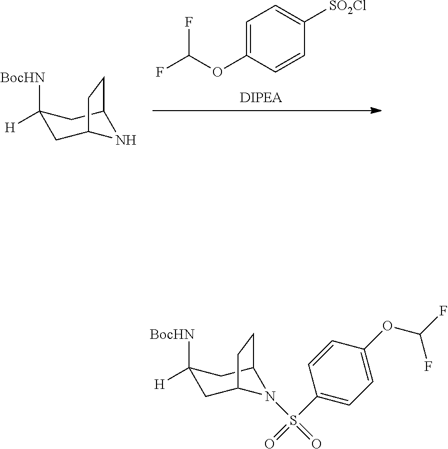 [SMILES: O=S(=O)(Cl)c1ccc(OC(F)F)cc1 ; CC(C)(C)OC(=O)NC1CC2CCC(C1)N2 ; CC(C)(C)OC(=O)NC1CC2CCC(C1)N2S(=O)(=O)c1ccc(OC(F)F)cc1]

To a solution of tert-butyl ((1R,3r,5S)-8-azabicyclo[3.2.1]octan-3-yl) carbamate (1.13 g, 5.0 mmol) and DIPEA (1.74 mL, 10.0 mmol) in DCM (25 mL) was added 4-(difluoromethoxy) benzenesulfonyl chloride (1.21 g, 5 mmol) and the mixture stirred at room temperature overnight. The organic phase was washed with sat. aq. NaHCO₃ and water, dried (MgSO₄), filtered, and evaporated to dryness in vacuo. The residue was purified by column chromatography (24 g SiO₂, 10-40% EtOAc in heptane) to give tert-butyl ((1R,3r,5S)-8-((4-(difluoromethoxy)phenyl)-sulfonyl)-8-azabicyclo[3.2.1]octan-3-yl) carbamate as a white solid (1.42 g, 66%). LCMS m/z=433.1 [M+H]⁺

### 2. Synthesis of (1R,3r,5S)-8-((4-(difluoromethoxy)phenyl)sulfonyl)-8-azabicyclo[3.2.1]octan-3-amine

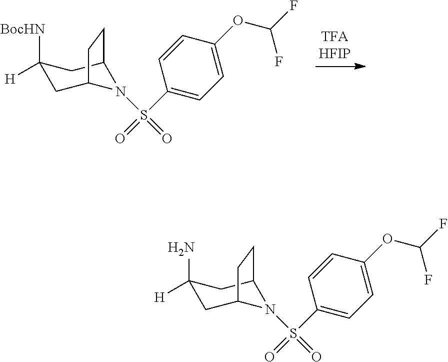 [SMILES: CC(C)(C)OC(=O)NC1CC2CCC(C1)N2S(=O)(=O)c1ccc(OC(F)F)cc1 ; NC1CC2CCC(C1)N2S(=O)(=O)c1ccc(OC(F)F)cc1]

TFA (200 μL, 2.71 mmol) was added dropwise at room temperature to a solution of tert-butyl ((1R,3r,5S)-8-((4-(difluoromethoxy)phenyl)sulfonyl)-8-azabicyclo[3.2.1]octan-3-yl) carbamate (468 mg, 1.1 mmol) in HFIP (3 mL) and the resulting mixture stirred at room temperature overnight. The reaction mixture was evaporated to dryness in vacuo and the co-evaporated with MeCN (×3) to afford (1R,3r,5S)-8-((4-resulting residue (difluoromethoxy)phenyl)sulfonyl)-8-azabicyclo[3.2.1]octan-3-amine as a white solid (543 mg, 100%). LCMS m/z=333.1 [M+H]⁺.

### Intermediate 4: (1R,3s,5S)-8-((4-(Difluoromethoxy)phenyl)sulfonyl)-8-azabicyclo[3.2.1]octan-3-amine

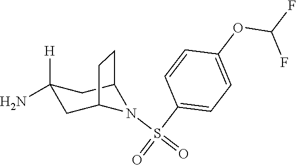 [SMILES: NC1CC2CCC(C1)N2S(=O)(=O)c1ccc(OC(F)F)cc1]

### 1. Synthesis of tert-butyl ((1R,3s,5S)-8-((4-(Difluoromethoxy)phenyl)sulfonyl)-8-azabicyclo[3.2.1]octan-3-yl)carbamate

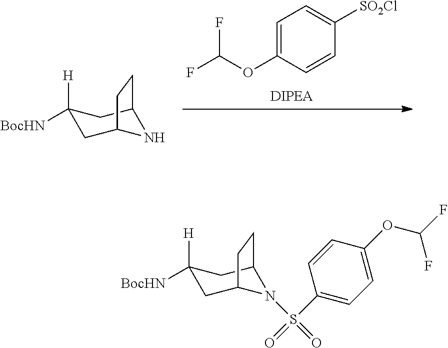 [SMILES: O=S(=O)(Cl)c1ccc(OC(F)F)cc1 ; CC(C)(C)OC(=O)NC1CC2CCC(C1)N2 ; CC(C)(C)OC(=O)NC1CC2CCC(C1)N2S(=O)(=O)c1ccc(OC(F)F)cc1]

To a solution of tert-butyl ((1R,3s,5S)-8-azabicyclo[3.2.1]octan-3-yl)carbamate (1.13 g, 5.0 mmol) and DIPEA (1.74 mL, 10.0 mmol) in DCM (25 mL) was added 4-(difluoromethoxy) benzenesulfonyl chloride (1.21 g, 5 mmol) and the mixture stirred at room temperature overnight. The mixture was diluted with EtOAc and washed with sat. aq. NaHCO₃ and water, dried (MgSO₄), filtered, and evaporated to dryness in vacuo. The resulting residue was triturated with MeCN to give tert-butyl ((1R,3s,5S)-8-((4-(difluoromethoxy)phenyl)-sulfonyl)-8-azabicyclo[3.2.1]octan-3-yl) carbamate as a white solid (1.88 g, 87%). LCMS m/z=433.1 [M+H]⁺.

### 2. Synthesis of (1R,3s,5S)-8-((4-(difluoromethoxy)phenyl)sulfonyl)-8-azabicyclo[3.2.1]octan-3-amine

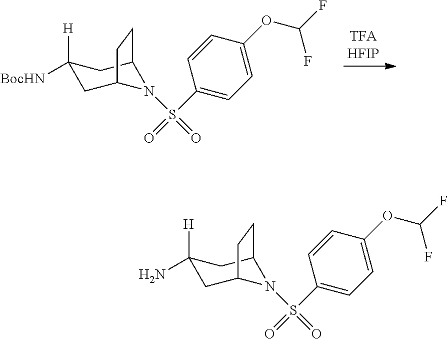 [SMILES: CC(C)(C)OC(=O)NC1CC2CCC(C1)N2S(=O)(=O)c1ccc(OC(F)F)cc1 ; NC1CC2CCC(C1)N2S(=O)(=O)c1ccc(OC(F)F)cc1]

TFA (220 μL, 2.9 mmol) was added dropwise at room temperature to a solution of tert-butyl ((1R,3s,5S)-8-((4-(difluoromethoxy)phenyl)sulfonyl)-8-azabicyclo[3.2.1]octan-3-yl) carbamate (468 mg, 1.1 mmol) in HFIP (3 mL), then the resulting mixture was stirred at room temperature for 3 h. The reaction mixture was evaporated to dryness in vacuo and the resulting residue was co-evaporated with MeCN (×3) to afford (1R,3s,5S)-8-((4-(difluoromethoxy)phenyl)sulfonyl)-8-azabicyclo[3.2.1]octan-3-amine trifluoroacetate as a white solid (521 mg, 100%). LCMS m/z=333.1 [M+H]⁺.

### Intermediate 5:8-((4-(Difluoromethoxy)phenyl)sulfonyl)-8-azabicyclo[3.2.1]octan-3-one

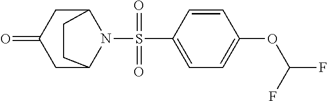 [SMILES: O=C1CC2CCC(C1)N2S(=O)(=O)c1ccc(OC(F)F)cc1]

To a mixture of (1S,5R)-8-azabicyclo[3.2.1]octan-3-one hydrochloride (666 mg, 4.1 mmol) and 4-(difluoromethoxy) benzenesulfonyl chloride (656 μL, 4.1 mmol) in DCM (8 mL) was added DIPEA (2.15 mL, 12.4 mmol). The reaction mixture was stirred at room temperature for 6 h. The organic phase was washed with sat. aq. NaHCO₃, water, dried (MgSO₄), filtered, and evaporated to dryness. The resulting residue was purified by column chromatography (80 g SiO₂, 10-40% EtOAc in heptane) to afford 8-((4-(difluoromethoxy)phenyl)sulfonyl)-8-azabicyclo[3.2.1]octan-3-one as a white solid (1.85 g, 79%). LCMS m/z=332.0 [M+H]⁺.

### Intermediate 6. 8-((2-Methyl-6-(trifluoromethyl)pyridin-3-yl)sulfonyl)-8-azabicyclo[3.2.1]octan-3-one

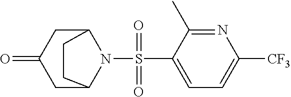 [SMILES: Cc1nc(C(F)(F)F)ccc1S(=O)(=O)N1C2CCC1CC(=O)C2]

DIPEA (92 μL, 0.5 mmol) was added dropwise to a solution of 8-azabicyclo[3.2.1]octan-3-one hydrochloride (177 mg, 1.1 mmol) and 2-methyl-6-(trifluoromethyl)pyridine-3-sulfonyl chloride (263 mg, 1.0 mmol) in DCM (5 mL) and the resulting mixture stirred at room temperature overnight.

The reaction mixture was diluted with EtOAc and the organic phase washed with sat. aq. NaHCO₃ and water, dried (MgSO₄), filtered, and evaporated to dryness in vacuo. The residue was purified by column chromatography (24 g SiO₂, 20-50% EtOAc in heptane) to afford 8-((2-methyl-6-(trifluoromethyl)pyridin-3-yl)sulfonyl)-8-azabicyclo[3.2.1]octan-3-one as a colorless oil (322 mg, 91%). LCMS m/z=349.1 [M+H]⁺.

### Intermediate 7. 4-(((1R,3s,5S)-8-Azabicyclo[3.2.1]octan-3-yl)methyl)morpholine

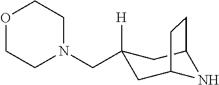 [SMILES: C1CN(CC2CC3CCC(C2)N3)CCO1]

### 1. Synthesis of tert-butyl (1R,3s,5S)-3-(morpholinomethyl)-8-azabicyclo[3.2.1]octane-8-carboxylate

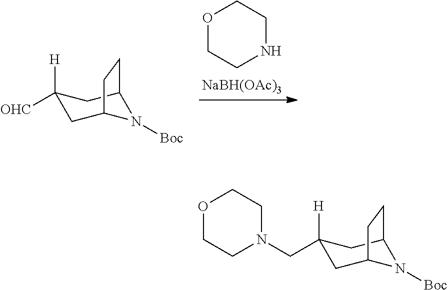 [SMILES: CC(C)(C)OC(=O)N1C2CCC1CC(C=O)C2 ; C1COCCN1 ; CC(C)(C)OC(=O)N1C2CCC1CC(CN1CCOCC1)C2 ; "NaBH(OAc)3"]

NaBH(OAc)₃ (36.54 g, 172 mmol) was added portion wise at room temperature to a solution of tert-butyl (1R,3s,5S)-3-formyl-8-azabicyclo[3.2.1]octane-8-carboxylate (27.5 g, 115 mmol) and morpholine (10.52 g, 121 mmol) in DCE (300 mL) and the resulting mixture stirred overnight at rt. MeOH (150 mL) was added, and the reaction mixture was evaporated to dryness. The residue was quenched with 15% potassium carbonate solution, extracted with EtOAc (2×250 mL). The combined organics were washed with water, dried (Na₂SO₄), filtered and evaporated in vacuo to afford tert-butyl (1R,3s,5S)-3-(morpholinomethyl)-8-azabicyclo[3.2.1]octane-8-carboxylate (27.4 g, 77%).

### 2. Synthesis of 4-(((1R,3s,5S)-8-azabicyclo[3.2.1]octan-3-yl)methyl)morpholine

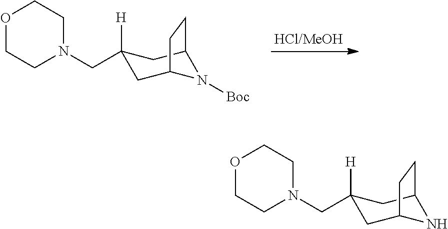 [SMILES: CC(C)(C)OC(=O)N1C2CCC1CC(CN1CCOCC1)C2 ; "HCl/MeOH" ; C1CN(CC2CC3CCC(C2)N3)CCO1]

tert-Butyl (1R,3s,5S)-3-(morpholinomethyl)-8-azabicyclo[3.2.1]octane-8-carboxylate (27.4 g, 88.3 mmol) was dissolved in HCl solution in MeOH (150 mL) and stirred overnight. The solvent was evaporated and THF (150 mL) added to the residue. The precipitate was collected by filtration and washed with THF (2×75 mL) and dried to afford 4-(((1R,3s,5S)-8-azabicyclo[3.2.1]octan-3-yl)methyl)morpholine dihydrochloride as a white solid (17.6 g, 70%). ¹H NMR (500 MHZ, DMSO-d₆) δ (ppm): 3.89 (dd, J=14.4, 10.3 Hz, 6H), 3.45 (d, J=12.6 Hz, 2H), 2.99 (q, J=12.6, 11.8 Hz, 2H), 2.91 (t, J=6.0 Hz, 2H), 2.33 (dt, J=11.8, 6.2 Hz, 1H), 1.96-1.83 (m, 6H), 1.63 (t, J=12.6 Hz, 2H).

### Intermediate 8: 4-(((1R,3r,5S)-8-Azabicyclo[3.2.1]octan-3-yl)methyl)morpholine

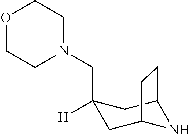 [SMILES: C1CN(CC2CC3CCC(C2)N3)CCO1]

4-(((1R,3r,5S)-8-Azabicyclo[3.2.1]octan-3-yl)methyl)morpholine was prepared from tert-butyl (1R,3r,5S)-3-formyl-8-azabicyclo[3.2.1]octane-8-carboxylate and morpholine using an analogous 2-step method as described for Intermediate 7. 1H NMR (400 MHZ, DMSO-d₆) δ (ppm): 4.02-3.82 (m, 6H), 3.47 (d, J=12.4 Hz, 2H), 3.08-2.85 (m, 4H), 2.36 (dd, J=11.4, 5.9 Hz, 1H), 2.00-1.82 (m, 6H), 1.71-1.58 (m, 2H). LCMS m/z=211.2 [M+H]⁺.

### Intermediate 9: (1R,3s,5S)-8-((2-Methyl-6-(trifluoromethyl)pyridin-3-yl)sulfonyl)-8-azabicyclo[3.2.1]octan-3-amine

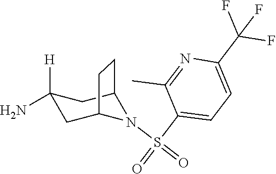 [SMILES: Cc1nc(C(F)(F)F)ccc1S(=O)(=O)N1C2CCC1CC(N)C2]

### 1. Synthesis of tert-butyl (1R,3s,5S)-3-(2,2,2-trifluoroacetamido)-8-azabicyclo[3.2.1]octane-8-carboxylate

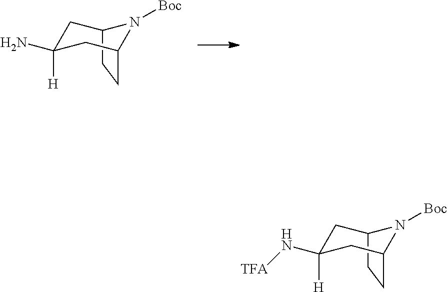

tert-Butyl (1R,3s,5S)-3-amino-8-azabicyclo[3.2.1]octane-8-carboxylate (2.5 g, 11 mmol) was dissolved in dry DCM (50 mL) and trimethylamine (1.11 g, 11 mmol) was added. The mixture was cooled to 0° C. and trifluoroacetic anhydride (2.31 g, 11 mmol) was added dropwise. The reaction mixture was warmed up to 20° C. and stirred for 18 h. The resulting mixture was washed with water (100 mL) and an aqueous NaHSO₄ solution (50 mL), dried over Na₂SO₄, filtered, and concentrated in vacuo to obtain tert-butyl (1R,3s,5S)-3-(2,2,2-trifluoroacetamido)-8-azabicyclo[3.2.1]octane-8-carboxylate (3.38 g, 95% yield) as a yellow oil.

### 2. Synthesis of N-((1R,3s,5S)-8-azabicyclo[3.2.1]octan-3-yl)-2,2,2-trifluoroacetamide

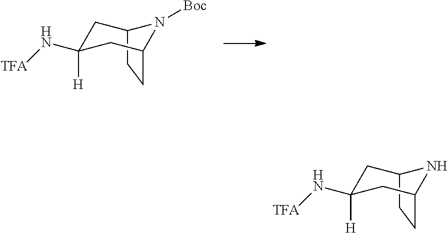

tert-Butyl (1R,3s,5S)-3-(2,2,2-trifluoroacetamido)-8-azabicyclo[3.2.1]octane-8-carboxylate (3.38 g, 10.5 mmol) was dissolved in 4 N HCl/dioxane solution and the reaction mixture was stirred for 4 h at 20° C. The volatiles were removed in vacuo and the residue was washed with diethyl ether (50 mL) and dried in vacuo to obtain N-((1R,3s,5S)-8-azabicyclo[3.2.1]octan-3-yl)-2,2,2-trifluoroacetamide (2.64 g, 97% yield) as a white solid.

### 3. Synthesis of 2,2,2-trifluoro-N-((1R,3s,5S)-8-((2-methyl-6-(trifluoromethyl)pyridin-3-yl)sulfonyl)-8-azabicyclo[3.2.1]octan-3-yl)acetamide

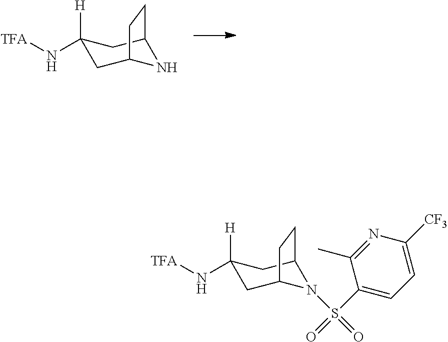

N-((1R,3s,5S)-8-Azabicyclo[3.2.1]octan-3-yl)-2,2,2-trifluoroacetamide (1 g, 4.5 mmol) was suspended in dry DCM (50 mL) and the mixture was cooled to 0° C. After that 2-methyl-6-(trifluoromethyl)pyridine-3-sulfonyl chloride (1 g, 3.9 mmol) was added in one portion followed by slow addition of DIPEA (1 g, 7.8 mmol). The reaction mixture was warmed up to 20° C. and stirred for 18 h at 20° C. The resulting mixture was washed with water (100 mL) and an aqueous NaHCO₃ solution (100 mL), dried over Na₂SO₄, and filtered through silica gel (100 mL) which was washed with MTBE (100 mL). Volatiles were removed in vacuo to obtain 2,2,2-trifluoro-N-((1R,3s,5S)-8-((2-methyl-6-(trifluoromethyl)pyridin-3-yl)sulfonyl)-8-azabicyclo[3.2.1]octan-3-yl)acetamide as a viscous yellow oil (1.7 g, 97% yield).

### 4. Synthesis of (1R,3s,5S)-8-((2-methyl-6-(trifluoromethyl)pyridin-3-yl)sulfonyl)-8-azabicyclo[3.2.1]octan-3-amine

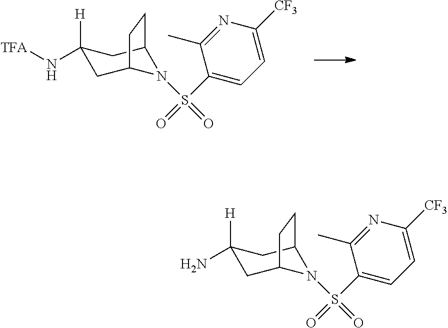

2,2,2-Trifluoro-N-((1R,3s,5S)-8-((2-methyl-6-(trifluoromethyl)pyridin-3-yl)sulfonyl)-8-azabicyclo[3.2.1]octan-3-yl)acetamide (1.7 g, 3.8 mmol) was dissolved in methanol (50 mL) and a solution of NaOH (0.76 g, 19 mmol) in water (10 mL) was added. The resulting mixture was stirred for 72 h at 20° C. Volatiles were removed in vacuo and the residue was diluted with an aqueous NaHSO₄ solution (250 mL) and washed with MTBE (100 mL). The aqueous layer was basified with NaOH and extracted with MTBE (3×100 mL). Combined organic layers were dried over Na₂SO₄, filtered, and concentrated in vacuo to obtain (1R,3s,5S)-8-((2-methyl-6-(trifluoromethyl)pyridin-3-yl)sulfonyl)-8-azabicyclo[3.2.1]octan-3-amine (1.3 g, 3.7 mmol, 98% yield) as a yellow oil. ¹H NMR (500 MHZ, Chloroform-d) δ 8.44 (d, J=8.0 Hz, 1H), 7.65 (d, J=8.1 Hz, 1H), 4.28-4.14 (m, 2H), 3.11 (tt, J=11.6, 5.9 Hz, 1H), 2.94 (d, J=1.7 Hz, 3H), 2.15-1.98 (m, 2H), 1.90 (ddd, J=13.5, 5.5, 2.8 Hz, 2H), 1.81-1.70 (m, 2H), 1.57-1.30 (m, 4H).

### Examples 1, 2 and 40

The title compounds were prepared using a single step library protocol as described below. Each reaction was carried out on an approximate 50 mg target product scale.

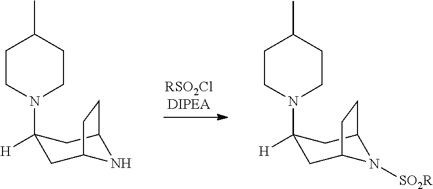

The appropriate sulfonyl chloride (RSO₂Cl, 1.1 equiv.) was added to a solution of (1R,3r,5S)-3-(4-methylpiperidin-1-yl)-8-azabicyclo[3.2.1]octane (Intermediate 1, 1.0 equiv.) and DIPEA (2.5 equiv.) in dry MeCN (1.2 mL) and the resulting mixture stirred at room temperature for 16 h. The solvent was evaporated in vacuo and the residue dissolved in DMSO (800 μL) and purified by prep-HPLC (YMC Actus Trial C18 20×100 mm, 5 μm; MeOH/water+0.1% NH₄OH) to afford the title compound. Gradient optimized for each compound.

| Example | |
|---|---|
| Number | Name/Structure/Sulfonyl chloride/HPLC/Data |
| 1 | 3-Methyl-4-(((1R,3r,5S)-3-(4-methylpiperidin-1-yl)- |
| | 8-azabicyclo[3.2.1]octan- |
| | 8-yl)sulfonyl)-1,2,5-oxadiazole |
| | 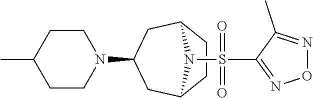 [SMILES: Cc1nonc1S(=O)(=O)N1[C@@H]2CC[C@H]1C[C@@H](N1CCC(C)CC1)C2] |
| | RSO₂Cl: 4-Methyl-1,2,5-oxadiazole-3-sulfonyl chloride |
| | HPLC (% organic): 60-100%; Yield: 4 mg (7%); |
| | LCMS m/z = 355.2 [M + H]⁺; |
| | t_{R} = 0.975 min. |
| 2 | 5-(((1R,3r,5S)-3-(4-Methylpiperidin-1-yl)- |
| | 8-azabicyclo[3.2.1]octan-8- |
| | yl)sulfonyl)-2-(tetrahydro-2H-pyran-4-yl)oxazole |
| | 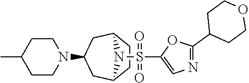 [SMILES: CC1CCN([C@H]2C[C@H]3CC[C@@H](C2)N3S(=O)(=O)c2cnc(C3CCOCC3)o2)CC1] |
| | RSO₂Cl: 2-(Tetrahydro-2H-pyran-4-yl)oxazole-5-sulfonyl |
| | chloride |
| | HPLC (% organic): 60-85%; Yield: 4 mg (7%); |
| | LCMS m/z = 424.4 [M + H]⁺; |
| | t_{R} = 0.882 min. |
| 40 | (1R,3r,5S)-3-(4-methylpiperidin-1-yl)-8- |
| | ((1-(4-methylpyridin-2-yl)-1H- |
| | pyrazol-4-yl)sulfonyl)-8-azabicyclo[3.2.1]octane |
| | 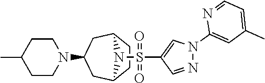 |
| | RSO₂Cl: 1-(4-methylpyridin-2-yl)-1H-pyrazole- |
| | 4-sulfonyl chloride |
| | HPLC (% organic): 40-90%; Yield: 3 mg (6%); |
| | LCMS m/z = 430.2 [M + H]⁺; t_{R} = 0.983 min. |

### Examples 3-13 and 41-47

### General Procedures B

The title compounds were prepared using a single step library protocol on an approximately 50 mg target product scale as described below.

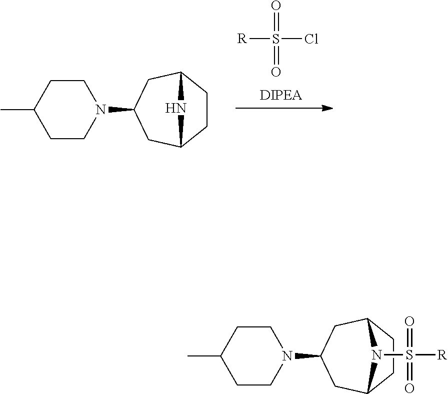

The appropriate sulfonyl chloride (RSO₂Cl) (1.1 equiv.) was added to the solution of (1R,3s,5S)-3-(4-methylpiperidin-1-yl)-8-azabicyclo[3.2.1]octane (Intermediate 2, 1.0 equiv.) and DIPEA (2.5 equiv.) in dry MeCN (1.2 mL) and the reaction mixture stirred at room temperature for 16 h. The solvent was evaporated to dryness in vacuo and the residue was dissolved in DMSO (800 μL) and purified by prep-HPLC to afford the title compound. Solvent gradient optimized for each compound.
Prep-HPLC-A: Waters SunFire C18 19×100 mm, 5 μm; water/MeOH
Prep-HPLC-B: YMC Actus Trial C18 20×100 mm, 5 μm; water/MeCN+0.1% NH₄OH

| Example | Name/Structure/Sulfonyl chloride |
|---|---|
| Number | HPLC gradient/Yield/Data |
| 3 | (1R,3s,5S)-8-((2-Methoxy-6-methylpyridin- |
| | 3-yl)sulfonyl)-3-(4-methylpiperidin- |
| | 1-yl)-8-azabicyclo[3.2.1]octane |
| | 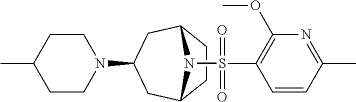 |
| | RSO₂Cl: 2-Methoxy-6-methylpyridine-3-sulfonyl chloride |
| | HPLC-B (% organic): 70-90%; Yield: 3 mg (6%); |
| | LCMS m/z = 394.0 [M + H]⁺; t_{R} = 0.997 min. |
| 4 | (1R,3s,5S)-8-((4-Chloro-1-(methoxymethyl)- |
| | 1H-pyrazol-5-yl)sulfonyl)-3-(4- |
| | methylpiperidin-1-yl)-8-azabicyclo[3.2.1]octane |
| | 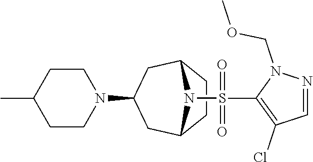 [SMILES: COCn1ncc(Cl)c1S(=O)(=O)N1[C@@H]2CC[C@H]1C[C@H](N1CCC(C)CC1)C2] |
| | RSO₂Cl: 4-Chloro-1-(methoxymethyl)-1H-pyrazole-5- |
| | sulfonyl chloride |
| | HPLC-B (% organic): 70-90%; Yield: 7.6 mg (18%); |
| | LCMS m/z = 417.2 [M + H]⁺; |
| | t_{R} = 0.858 min. |
| 5 | 3,3-Dimethyl-6-(((1R,3s,5S)-3-(4-methylpiperidin- |
| | 1-yl)-8-azabicyclo[3.2.1]octan- |
| | 8-yl)sulfonyl)-3,4-dihydroquinolin-2(1H)-one |
| | 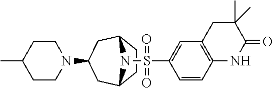 [SMILES: CC1CCN([C@@H]2C[C@H]3CC[C@@H](C2)N3S(=O)(=O)c2ccc3c(c2)CC(C)(C)C(=O)N3)CC1] |
| | RSO₂Cl: 3,3-Dimethyl-2-oxo-1,2,3,4-tetrahydroquinoline- |
| | 6-sulfonyl chloride |
| | HPLC-B (% organic): 45-75%; Yield: 8.6 mg (19%); |
| | ¹H NMR (500 MHz, DMSO-d₆) δ (ppm): 10.41 (s, 1H), |
| | 7.69 − 7.55 (m, 2H), 6.94 (d, J = 8.1 Hz, 1H), |
| | 4.14 (br s, 2H), 2.82 (s, 2H), 2.75 − 2.61 (m, 3H), |
| | 2.02 (br s, 2H), 1.71 − 1.38 (m, 9H), 1.29 − 1.19 (m, 3H), |
| | 1.03 (s, 6H), 0.83 (d, J = 6.4 Hz, 3H). LCMS m/z = |
| | 446.1 [M + H]⁺; t_{R} = 0.983 min. |
| 6 | 5-(((1R,3s,5S)-3-(4-Methylpiperidin-1- |
| | yl)-8-azabicyclo[3.2.1]octan-8- |
| | yl)sulfonyl)-2-(tetrahydro-2H-pyran-4-yl)oxazole |
| | 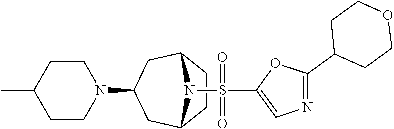 [SMILES: CC1CCN([C@@H]2C[C@H]3CC[C@@H](C2)N3S(=O)(=O)c2cnc(C3CCOCC3)o2)CC1] |
| | RSO₂Cl: 2-(Tetrahydro-2H-pyran-4-yl)oxazole-5- |
| | sulfonyl chloride |
| | HPLC-B (% organic): 50-75%; Yield: 1.2 mg (3%); |
| | LCMS m/z = 424.0 [M + H]⁺; t_{R} = 0.927 min. |
| 7 | (1R,3s,5S)-8-(Isochroman-7-ylsulfonyl)-3-(4-methylpiperidin- |
| | 1-yl)-8-azabicyclo[3.2.1]octane |
| | 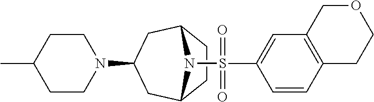 [SMILES: CC1CCN([C@@H]2C[C@H]3CC[C@@H](C2)N3S(=O)(=O)c2ccc3c(c2)COCC3)CC1] |
| | RSO₂Cl: Isochromane-7-sulfonyl chloride |
| | HPLC-B (% organic): 60-70%; Yield: 9.5 mg (23%); |
| | LCMS m/z = 405.0 [M + H]⁺; t_{R} = 0.970 min. |
| 8 | (1R,3s,5S)-8-((5-(Methoxymethyl)furan-2-yl)sulfonyl)-3- |
| | (4-methylpiperidin-1-yl)-8-azabicyclo[3.2.1]octane |
| | 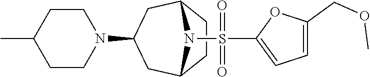 |
| | RSO₂Cl: 5-(Methoxymethyl)furan-2-sulfonyl chloride |
| | HPLC-B (% organic): 55-85%; Yield: 7 mg (18%); LCMS |
| | m/z = 383.1 [M + H]⁺; t_{R} = 0.938 min. |
| 9 | 2-Methyl-3-(((1R,3s,5S)-3-(4-methylpiperidin-1- |
| | yl)-8-azabicyclo[3.2.1]octan-8- |
| | yl)sulfonyl)-2,4,6,7-tetrahydropyrano[4,3-c]pyrazole |
| | 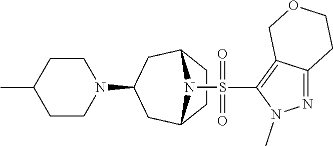 [SMILES: CC1CCN([C@@H]2C[C@H]3CC[C@@H](C2)N3S(=O)(=O)c2c3c(nn2C)CCOC3)CC1] |
| | RSO₂Cl: 2-Methyl-2,4,6,7-tetrahydropyrano[4,3-c] |
| | pyrazole-3-sulfonyl chloride |
| | HPLC-B (% organic): 50-70%; Yield: 6 mg (14%); |
| | LCMS m/z = 409.4 [M + H]⁺; t_{R} = 0.716 min. |
| 10 | (1R,3s,5S)-3-(4-Methylpiperidin-1-yl)-8-((3-phenyl- |
| | 1H-pyrazol-5-yl)sulfonyl)-8-azabicyclo[3.2.1]octane |
| | 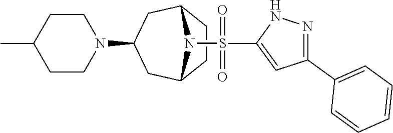 [SMILES: CC1CCN([C@@H]2C[C@H]3CC[C@@H](C2)N3S(=O)(=O)c2cc(-c3ccccc3)n[nH]2)CC1] |
| | RSO₂Cl: 3-Phenyl-1H-pyrazole-5-sulfonyl chloride |
| | HPLC-B (% organic): 45-75%; Yield: 6.3 mg (14%); |
| | LCMS m/z = 415.2 [M + H]⁺; t_{R} = 0.977 min. |
| 11 | 5-Methyl-2-(((1R,3s,5S)-3-(4-methylpiperidin-1-yl)- |
| | 8-azabicyclo[3.2.1]octan-8-yl)sulfonyl)-5,6,7,8- |
| | tetrahydroimidazo[1,2-a]pyridine |
| | 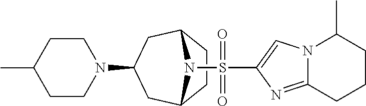 [SMILES: CC1CCN([C@@H]2C[C@H]3CC[C@@H](C2)N3S(=O)(=O)c2cn3c(n2)CCCC3C)CC1] |
| | RSO₂Cl: 5-Methyl-5,6,7,8-tetrahydroimidazo[1,2-a] |
| | pyridine-2-sulfonyl chloride |
| | HPLC-A (% organic): 45-75%; Yield: 7.9 mg (16%); |
| | ¹H NMR (400 MHz, acetonitrile-d₃) δ (ppm): 7.51 (s, |
| | 1H), 4.21 (br d, J = 5.3 Hz, 3H), 2.87 − 2.65 (m, |
| | 5H), 2.29 (br s, 2H), 2.19 − 2.04 (m, 4H), |
| | 1.87 − 1.53 (m, 10H), 1.47 (d, J = 6.5 |
| | Hz, 3H), 1.37 − 1.24 (m, 1H), 1.16 − 1.02 |
| | (m, 2H), 0.91 (d, J = 6.5 Hz, 3H). |
| | LCMS m/z = 407.2 [M + H]⁺; t_{R} = 0.952 min. |
| 12 | (1R,3s,5S)-8-((1,4-Dimethyl-1H-pyrazol-5-yl) |
| | sulfonyl)-3-(4-methylpiperidin-1- |
| | yl)-8-azabicyclo[3.2.1]octane |
| | 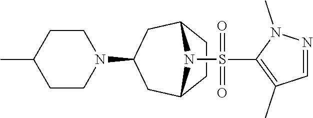 [SMILES: Cc1cnn(C)c1S(=O)(=O)N1[C@@H]2CC[C@H]1C[C@H](N1CCC(C)CC1)C2] |
| | RSO₂Cl: 1,4-Dimethyl-1H-pyrazole-5-sulfonyl chloride |
| | HPLC-A (% organic): 45-75%; Yield: 9 mg (18%); |
| | LCMS m/z = 367.2 [M + H]⁺; |
| | t_{R} = 0.935 min. |
| 13 | (1R,3s,5S)-8-((1-Ethyl-1H-pyrazol-5-yl)sulfonyl)- |
| | 3-(4-methylpiperidin-1-yl)-8-azabicyclo[3.2.1]octane |
| | 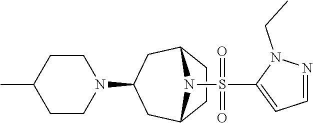 [SMILES: CCn1nccc1S(=O)(=O)N1[C@@H]2CC[C@H]1C[C@H](N1CCC(C)CC1)C2] |
| | RSO₂Cl: 1-Ethyl-1H-pyrazole-5-sulfonyl chloride |
| | HPLC: Waters Sunfire Prep C18 30 × 100 mm, 5 μm; |
| | 5-40% MeCN/water + TFA Yield: 28.9 mg (66%); |
| | ¹H NMR (500 MHz, DMSO-d₆) δ (ppm): 8.98 − 8.80 (m, |
| | 1H), 8.32 (s, 1H), 4.46 (spt, J = 6.6 Hz, 1H), 4.27 (br d, |
| | J = 9.2 Hz, 2H), 2.95 − 2.81 (m, 2H), 2.68 (td, J = 8.5, |
| | 13.4 Hz, 2H), 2.53 − 2.51 (m, 2H), 2.29 (s, 2H), |
| | 1.81 (br d, J = 13.4 Hz, 2H), 1.52 (s, 7H), 1.38 (d, J = |
| | 6.7 Hz, 3H), 1.32 − 1.21 (m, 2H), 0.90 (d, J = 6.7 Hz, 3H). |
| | LCMS m/z = 367.2 [M + H]⁺; t_{R} = 1.31 min. |
| 41 | (1R,3s,5S)-3-(4-Methylpiperidin-1-yl)-8-((1- |
| | (4-methylpyridin-2-yl)-1H-pyrazol- |
| | 4-yl)sulfonyl)-8-azabicyclo[3.2.1]octane |
| | 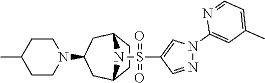 |
| | RSO₂Cl: 1-(4-methylpyridin-2-yl)-1H-pyrazole- |
| | 4-sulfonyl chloride |
| | HPLC-B (% organic): 45-60%; Yield: |
| | 8 mg (14%); LCMS m/z = 430.2 [M + H]⁺; |
| | t_{R} = 1.131 min. |
| 42 | 4-(((1R,3s,5S)-3-(4-Methylpiperidin-1-yl)-8-azabicyclo |
| | [3.2.1]octan-8-yl)sulfonyl)-2-(trifluoromethyl)thiazole |
| | 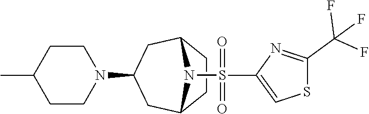 [SMILES: CC1CCN([C@@H]2C[C@H]3CC[C@@H](C2)N3S(=O)(=O)c2csc(C(F)(F)F)n2)CC1] |
| | RSO₂Cl: 2-(trifluoromethyl)thiazole-4-sulfonyl chloride |
| | HPLC-B (% organic): 55-85%; Yield: 14 mg (28%); |
| | LCMS m/z = 424.2 [M + H]⁺; t_{R} = 1.036 min. |
| 43 | (1R,3s,5S)-8-((2,6-Difluoro-4-methoxyphenyl) |
| | sulfonyl)-3-(4-methylpiperidin-1- |
| | yl)-8-azabicyclo[3.2.1]octane |
| | 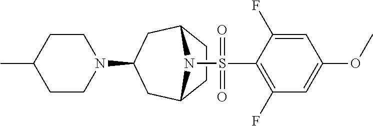 |
| | RSO₂Cl: 2,6-difluoro-4-methoxybenzenesulfonyl chloride |
| | HPLC-A (% organic): 45-75%; Yield: 10 mg (20%); |
| | LCMS m/z = 415.2 [M + H]⁺; |
| | t_{R} = 0.844 min. |
| 44 | (1R,3s,5S)-3-(4-Methylpiperidin-1- |
| | yl)-8-((1-phenyl-1H-1,2,3-triazol-4- |
| | yl)sulfonyl)-8-azabicyclo[3.2.1]octane |
| | 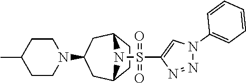 [SMILES: CC1CCN([C@@H]2C[C@H]3CC[C@@H](C2)N3S(=O)(=O)c2cn(-c3ccccc3)nn2)CC1] |
| | RSO₂Cl: 1-phenyl-1H-1,2,3-triazole-4-sulfonyl chloride |
| | HPLC-B (% organic): 50-80%; Yield: 1 mg (2%); |
| | LCMS m/z = 416.4 [M + H]⁺; t_{R} = 0.969 min. |
| 45 | 1,3-Dimethyl-5-(((1R,3s,5S)-3-(4-methylpiperidin-1-yl)-8- |
| | azabicyclo[3.2.1]octan-8-yl)sulfonyl)-1H-pyrazolo[3,4-b] |
| | pyridine |
| | 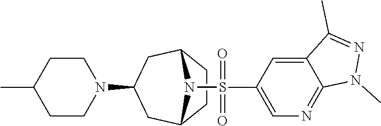 |
| | RSO₂Cl: 1,3-dimethyl-1H-pyrazolo[3,4-b]pyridine-5-sulfonyl |
| | chloride |
| | HPLC-A (% organic): 45-75%; Yield: 7 mg (14%); LCMS |
| | m/z = 418.0 [M + H]⁺; t_{R} = 0.821 min. |
| 46 | (1R,3s,5S)-8-((3,5-Dimethylpyridin-2-yl)sulfonyl)-3- |
| | (4-methylpiperidin-1-yl)-8-azabicyclo[3.2.1]octane |
| | 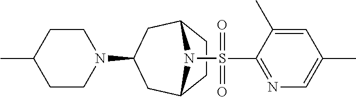 |
| | RSO₂Cl: 3,5-dimethylpyridine-2-sulfonyl chloride |
| | Yield: 30 mg (9%); ¹H NMR (500 MHz, DMSO-d₆) δ (ppm): |
| | 8.32 (s, 1H), 7.71 (s, 1H), 4.42-4.07 (m, 4H), 2.93-2.69 (m, 3H), |
| | 2.54 (s, 3H), 2.34 (s, 3H), 2.14-1.86 (m, 1H), 1.82-1.52 |
| | (m, 9H), 1.38-1.18 (m, 3H), 0.88 (s, 3H); LCMS m/z = |
| | 378.2 [M + H]⁺. |
| 47 | (1R,3s,5S)-8-((2,6-Dimethylpyridin-3-yl)sulfonyl)-3- |
| | (4-methylpiperidin-1-yl)-8-azabicyclo[3.2.1]octane |
| | 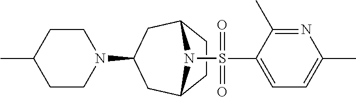 |
| | RSO₂Cl: 2,6-dimethylpyridine-3-sulfonyl chloride |
| | HPLC: Column: Boston Prime C18 150*30 mm*5 um; Condition: |
| | water(0.05%NH₃H₂O + 10 mM NH₄HCO₃)-ACN; from 52% to |
| | 82%; Flow Rate: 25 mL/min); Yield: 15 mg (8%); |
| | ¹H NMR: (500 MHz, METHANOL-d4) δ = |
| | 8.20 (d, J = 8.0 Hz, 1H), 7.29 (d, J = 8.0 Hz, 1H), 4.25 − 4.14 |
| | (m, 2H), 2.90 (br d, J = 11.5 Hz, 2H), 2.82 (s, 3H), 2.74 − 2.63 |
| | (m, 1H), 2.57 (s, 3H), 2.13 − 2.04 (m, 2H), 2.01 − 1.94 |
| | (m, 2H), 1.90 − 1.85 (m, 2H), 1.79 − 1.73 (m, 2H), 1.67 − |
| | 1.61 (m, 4H), 1.41 − 1.32 (m, 1H), 1.23 − 1.13 (m, 2H), 0.91 |
| | (d, J = 6.5 Hz, 3H); LCMS m/z = 378.2.0 [M + H]⁺. |

### Examples 14-16 and 48-58

General procedure for reductive amination of primary (RNH₂) and secondary amines (R¹R²NH)

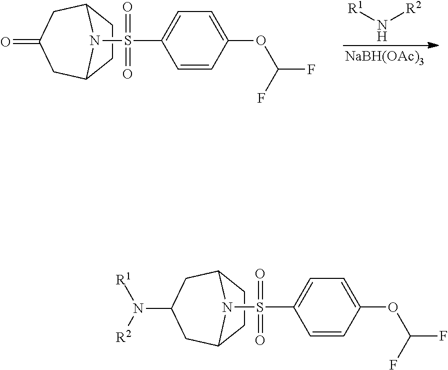

The title compounds were prepared using a single step reductive amination protocol on an approximately 50 mg target product scale using either the Primary Amine or Secondary Amine protocols outlined below.

Primary Amine Protocol: The appropriate primary amine (RNH₂, 1.3 equiv.), 8-((4-(difluoromethoxy)phenyl)sulfonyl)-8-azabicyclo[3.2.1]octan-3-one (Intermediate 5, 1.0 equiv.) and NaBH(OAc)₃ (4.0 equiv.) were dissolved in CHCl₃ (1.2 mL) and AcOH (1.2 equiv.), then the mixture stirred at room temperature for 24 h. The reaction mixture was concentrated under reduced pressure, and the residue was taken up in 5% ammonia in MeOH (1.2 mL) and stirred at room temperature for 0.5 h. The mixture was concentrated under reduced pressure and the residue was dissolved in the DMSO (0.7 mL) and purified by prep-HPLC (YMC Actus Triart C18 20×100 mm, 5 μm; gradient mixture water-MeOH+0.1% NH₄OH) to afford the title compound. In the case of using primary amines as HCl salts, an additional 1.4 equiv. DIPEA per each equivalent of acid was added to the reaction mixture. The molar ratios of the starting materials and overall reaction conditions were unchanged.

Secondary Amine Protocol: NaBH(OAc)₃ (3.0 equiv.) was added to a solution of 8-((4-(difluoromethoxy)phenyl)sulfonyl)-8-azabicyclo[3.2.1]octan-3-one (Intermediate 5, 1.0 equiv.) and the appropriate secondary amine (R¹R²NH, 1.1 equiv.) in 1,2-dichloroethane (1 mL) and the resulting solution stirred at 50° C. for 16 h. The reaction mixture was concentrated under reduced pressure, and the residue was taken up in sat. aq. NaHCO₃ and extracted with EtOAc (2×10 mL). The combined organics were washed with water (10 mL), brine (10 mL), dried (Na₂SO₄) and evaporated to dryness in vacuo. The residue was dissolved in DMSO (500 μL) and purified by prep-HPLC (YMC Actus Triart C18 20×100 mm, 5 μm; gradient mixture water-MeOH+0.1% NH₄OH) to afford the title compounds.

| Example | |
|---|---|
| Number | Name/Structure/Amine/HPLC/Data |
| 14 | 2-((8-((4-(Difluoromethoxy)phenyl)sulfonyl)-8-azabicyclo |
| | [3.2.1]octan-3-yl)amino)cyclobutan-1-ol |
| | 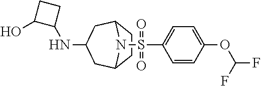 [SMILES: O=S(=O)(c1ccc(OC(F)F)cc1)N1C2CCC1CC(NC1CCC1O)C2] |
| | RNH₂: 2-Aminocyclobutan-1-ol hydrochloride |
| | HPLC (% organic): 40-70%; Yield: 10.6 mg (21%, 7:3 |
| | mixture of isomers). |
| | ¹H NMR (400 MHz, CD₃OD) δ (ppm): 7.97 − 7.89 (m, 2H), |
| | 7.35 − 7.27 (m, 2H), 7.23 − 6.79 (m, 1H), 4.35 − 4.12 |
| | (m, 2H), 3.74 (q, J = 7.7 Hz, 1H), 3.10 − 2.88 (m, 2H), 2.17 − |
| | 1.63 (m, 8H), 1.62 − 0.99 (m, 4H). LCMS m/z = 403.2 |
| | [M + H]⁺; t_{R} = 0.959 min. |
| 15 | 8-((4-(Difluoromethoxy)phenyl)sulfonyl)-N-((3-methyloxetan- |
| | 3-yl)methyl)-8-azabicyclo[3.2.1]octan-3-amine |
| | 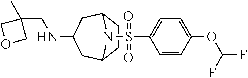 [SMILES: CC1(CNC2CC3CCC(C2)N3S(=O)(=O)c2ccc(OC(F)F)cc2)COC1] |
| | RNH₂: (3-Methyloxetan-3-yl)methanamine hydrochloride |
| | HPLC (% organic): 45-75%; Yield: 23.1 mg (46%, 7:3 |
| | mixture of isomers). |
| | ¹H NMR (400 MHz, DMSO-d₆ + CCl₄) δ (ppm): 7.93 − 7.78 |
| | (m, 2H), 7.42 − 6.99 (m, 3H), 4.29 (d, J = 5.8 |
| | Hz, 2H), 4.22 − 4.05 (m, 4H), 2.87 (s, 1H), 2.63 |
| | (br s, 2H), 2.07 − 1.70 (m, 5H), 1.58 − 1.15 (m, 7H). |
| | LCMS m/z = 417.2 [M + H]⁺; t_{R} = 0.908 min. |
| 16 | 9-(8-((4-(Difluoromethoxy)phenyl)sulfonyl)-8-azabicyclo |
| | [3.2.1]octan-3-yl)-3,3-difluoro-9-azabicyclo[3.3.1]nonane |
| | 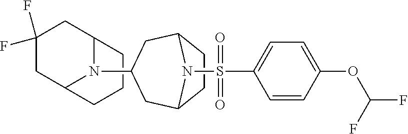 [SMILES: O=S(=O)(c1ccc(OC(F)F)cc1)N1C2CCC1CC(N1C3CCCC1CC(F)(F)C3)C2] |
| | R¹R²NH: 3,3-Difluoro-9-azabicyclo[3.3.1]nonane |
| | HPLC (% organic): 40-90%; Yield: 0.6 mg (4%, |
| | 7:3 mixture of isomers); |
| | LCMS m/z = 477.2 [M + H]⁺; t_{R} = 0.991 min. |
| 48 | 1-((8-((4-(Difluoromethoxy)phenyl)sulfonyl)-8- |
| | azabicyclo[3.2.1]octan-3-yl)amino)propan-2-ol |
| | 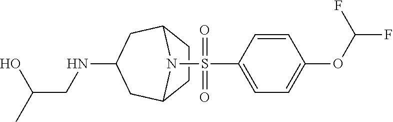 [SMILES: CC(O)CNC1CC2CCC(C1)N2S(=O)(=O)c1ccc(OC(F)F)cc1] |
| | R¹R²NH: 1-aminopropan-2-ol |
| | HPLC (% organic): 40-90%; Yield: 6.4 mg (13%); |
| | LCMS m/z = 391.2 |
| | [M + H]⁺; t_{R} = 0.693 min |
| 49 | 1-(8-((4-(Difluoromethoxy)phenyl)sulfonyl)-8-azabicyclo |
| | [3.2.1]octan-3-yl)-3-methylpyrrolidin-3-ol |
| | 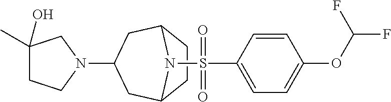 [SMILES: CC1(O)CCN(C2CC3CCC(C2)N3S(=O)(=O)c2ccc(OC(F)F)cc2)C1] |
| | R¹R²NH: 3-methylpyrrolidin-3-ol |
| | HPLC (% organic): 40-90%; Yield: 76 mg (60%); LCMS m/z = |
| | 417.2 [M + H]⁺; t_{R} = 1.586 min |
| 50 | 8-((4-(Difluoromethoxy)phenyl)sulfonyl)-3-(3-methoxypyrrolidin- |
| | 1-yl)-8-azabicyclo[3.2.1]octane |
| | 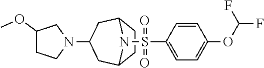 [SMILES: COC1CCN(C2CC3CCC(C2)N3S(=O)(=O)c2ccc(OC(F)F)cc2)C1] |
| | R¹R²NH: 3-methoxypyrrolidine |
| | HPLC (column: XBridge BEH C18 5um 130 A; |
| | 40-40-90% 0-1-6 min |
| | H2O/MeOH/0.1%NH4OH, flow: 30ml/min); Yield: 21 mg (33%); |
| | LCMS m/z = 417.2 [M + H]⁺; t_{R} = 2.389 min |
| 51 | 6-(8-((4-(Difluoromethoxy)phenyl)sulfonyl)-8-azabicyclo[3.2.1] |
| | octan-3-yl)-2-oxa-6-azaspiro[3.4]octane |
| | 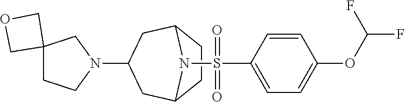 [SMILES: O=S(=O)(c1ccc(OC(F)F)cc1)N1C2CCC1CC(N1CCC3(COC3)C1)C2] |
| | R¹R²NH: 2-oxa-6-azaspiro[3.4]octane |
| | HPLC (% organic): 40-90%; Yield: 36 mg (26%); LCMS m/z = |
| | 429.2 [M + H]⁺; t_{R} = 1.959 min |
| 52 | 8-((4-(Difluoromethoxy)phenyl)sulfonyl)-N-(4-methoxybutan- |
| | 2-yl)-8-azabicyclo[3.2.1]octan-3-amine |
| | 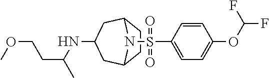 [SMILES: COCCC(C)NC1CC2CCC(C1)N2S(=O)(=O)c1ccc(OC(F)F)cc1] |
| | R¹R²NH: 4-methoxybutan-2-amine |
| | HPLC (% organic): 55-75%; Yield: 8 mg (12%); LCMS m/z = |
| | 419.0 [M + H]⁺; t_{R} = 0.874 min |
| 53 | 8-((4-(Difluoromethoxy)phenyl)sulfonyl)-8,8′-diaza-3,8′- |
| | bi(bicyclo[3.2.1]octane) |
| | 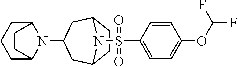 [SMILES: O=S(=O)(c1ccc(OC(F)F)cc1)N1C2CCC1CC(N1C3CCCC1CC3)C2] |
| | R¹R²NH: 8-((4-(difluoromethoxy)phenyl)sulfonyl)-8,8′-diaza- |
| | 3,8′-bi(bicyclo[3.2.1]octane) |
| | HPLC (% organic): 40-90%; Yield: 1.4 mg (1%); LCMS m/z = |
| | 427.2 [M + H]⁺; t_{R} = 0.916 min |
| 54 | 8-((4-(Difluoromethoxy)phenyl)sulfonyl)-3-(3-(2- |
| | methoxyethyl)azetidin-1-yl)-8-azabicyclo[3.2.1]octane |
| | 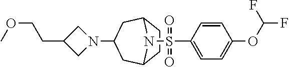 [SMILES: COCCC1CN(C2CC3CCC(C2)N3S(=O)(=O)c2ccc(OC(F)F)cc2)C1] |
| | R¹R²NH: 3-(2-methoxyethyl)azetidine |
| | Yield: 46 mg (35%); LCMS m/z = 431.2 [M + H]⁺; |
| | t_{R} = 0.946 min |
| 55 | 8-((4-(Difluoromethoxy)phenyl)sulfonyl)-N-(3-methoxybutan- |
| | 2-yl)-8-azabicyclo[3.2.1]octan-3-amine |
| | 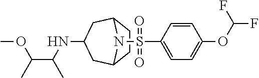 [SMILES: COC(C)C(C)NC1CC2CCC(C1)N2S(=O)(=O)c1ccc(OC(F)F)cc1] |
| | R¹R²NH: 3-methoxybutan-2-amine |
| | HPLC (% organic): 40-90%; Yield: 16 mg (32%); LCMS m/z = |
| | 419.0 [M + H]⁺; t_{R} = 0.800 min |
| 56 | rac-((1R,2S)-2-(((8-((4-(Difluoromethoxy)phenyl)sulfonyl)-8- |
| | azabicyclo[3.2.1]octan-3-yl)amino)methyl)cyclopropyl) |
| | methanol |
| | 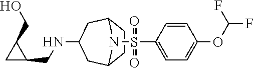 [SMILES: O=S(=O)(c1ccc(OC(F)F)cc1)N1C2CCC1CC(NC[C@H]1C[C@H]1CO)C2] |
| | R¹R²NH: rac-((1R,2S)-2-((11-azaneyl)methyl)cyclopropyl) |
| | methanol |
| | HPLC (% organic): 40-70%; Yield: 28 mg (32%); LCMS m/z = |
| | 417.1 [M + H]⁺; t_{R} = 1.008 min |
| 57 | 3-(8-((4-(Difluoromethoxy)phenyl)sulfonyl)-8-azabicyclo[3.2.1] |
| | octan-3-yl)-3-azabicyclo[3.1.1]heptane |
| | 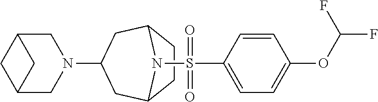 [SMILES: O=S(=O)(c1ccc(OC(F)F)cc1)N1C2CCC1CC(N1CC3CC(C3)C1)C2] |
| | R¹R²NH: 3-azabicyclo[3.1.1]heptane |
| | HPLC (% organic): 40-90%; Yield: 25 mg (20%); LCMS |
| | m/z = 413.2 [M + H]⁺; t_{R} = 2.662 min |
| 58 | 8′-((4-(Difluoromethoxy)phenyl)sulfonyl)-3,8′-diaza-3,3′- |
| | bi(bicyclo[3.2.1]octane) |
| | 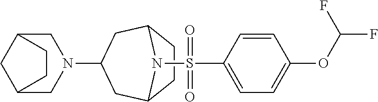 [SMILES: O=S(=O)(c1ccc(OC(F)F)cc1)N1C2CCC1CC(N1CC3CCC(C3)C1)C2] |
| | R¹R²NH: 3-azabicyclo[3.2.1 ]octane |
| | HPLC (% organic): 40-90%; Yield: 38 mg (30%); LCMS |
| | m/z = 427.4 [M + H]⁺; t_{R} = 2.652 min |

### Example 17: 4-((((1R,3r,5S)-8-((4-(Difluoromethoxy)phenyl)sulfonyl)-8-azabicyclo[3.2.1]octan-3-yl)amino)methyl)tetrahydro-2H-pyran-4-ol

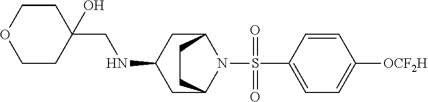

To a mixture of 1,6-dioxaspiro[2.5]octane (11 mg, 0.1 mmol) and (1R,3r,5S)-8-((4-(difluoromethoxy)phenyl)sulfonyl)-8-azabicyclo[3.2.1]octan-3-amine (Intermediate 3, 41 mg, 0.1 mmol) in EtOH (2 mL) was added DIPEA (36 μL, 0.2 mmol), and the mixture stirred at 60° C. overnight. Additional 1,6-dioxaspiro[2.5]octane was added then continued heating at 60° C. overnight. The reaction mixture was evaporated to dryness and dissolved in DMSO and purified by prep-HPLC (Waters Xselect CSH Prep C18 30×100 mm, 5 μm; 5-55% MeCN in water (+NH₄OH)) to afford 4-((((1R,3r,5S)-8-((4-(difluoromethoxy)phenyl)sulfonyl)-8-azabicyclo[3.2.1]octan-3-yl)amino)methyl)tetrahydro-2H-pyran-4-ol as a white solid (16 mg, 44%). LCMS m/z=447.1 [M+H]⁺, t_{R}=0.56 min.

### Example 18: 4-((((1R,3s,5S)-8-((4-(Difluoromethoxy)phenyl)sulfonyl)-8-azabicyclo[3.2.1]octan-3-yl)amino)methyl)tetrahydro-2H-pyran-4-ol

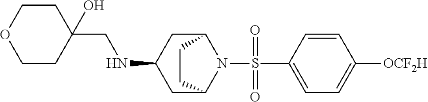

The title compound was prepared as a white solid (7.1 mg, 18%) from 1,6-dioxaspiro[2.5]octane and (1R,3s,5S)-8-((4-(difluoromethoxy)phenyl)sulfonyl)-8-azabicyclo[3.2.1]octan-3-amine (Intermediate 4) using an analogous procedure to that described for Example 17 to afford the desired compound (16 mg, 44%). LCMS m/z=446.8 [M+H]⁺, t_{R}=0.55 min.

### Example 19: 8-((4-(Difluoromethoxy)phenyl)sulfonyl)-N-((tetrahydro-2H-pyran-4-yl)methyl)-8-azabicyclo[3.2.1]octan-3-amine

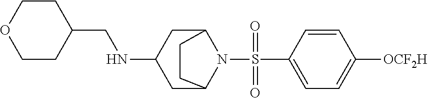

Acetic acid (34 μL, 0.6 mmol) was added to a mixture of tetrahydropyran-4-ylmethanamine (28 mg. 0.2 mmol) and 8-((4-(difluoromethoxy)phenyl)sulfonyl)-8-azabicyclo[3.2.1]octan-3-one (Intermediate 5, 66 mg. 0.2 mmol) in DCM (3 mL). NaBH(OAc)₃ (170 mg. 0.8 mmol) was added in one portion to the above solution, then the mixture was stirred at room temperature overnight. The reaction was quenched with sat. aq. NH₄Cl and stirred at room temperature for 5 min. The mixture was diluted with DCM and sat. aq. NaHCO₃ then stirred for another 5 min. The organic phase was washed with water and evaporated to dryness in vacuo. The residue was dissolved in DMSO purified by prep-HPLC (Waters Xselect CSH Prep C18 30×100 mm, 5 μm; 5-70% MeCN in water (+NH₄OH)) to afford 8-((4-(difluoromethoxy)phenyl)sulfonyl)-N-((tetrahydro-2H-pyran-4-yl)methyl)-8-azabicyclo[3.2.1]octan-3-amine as a white solid (24 mg, 28%). LCMS m/z=431.2 [M+H]⁺, t_{R}=0.58 min.

### Example 20 and 21: (1R,3r,5S)-8-((4-(difluoromethoxy)phenyl)sulfonyl)-3-(4-methylpiperidin-1-yl)-8-azabicyclo[3.2.1]octane and (1R,3s,5S)-8-((4-(difluoromethoxy)phenyl)sulfonyl)-3-(4-methylpiperidin-1-yl)-8-azabicyclo[3.2.1]octane

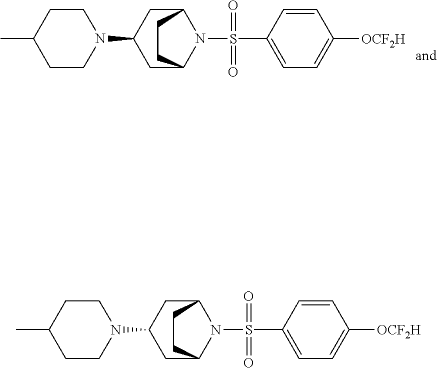

### 1. Synthesis of 8-((4-(difluoromethoxy)phenyl)sulfonyl)-3-(4-methylpiperidin-1-yl)-8-azabicyclo[3.2.1]octane

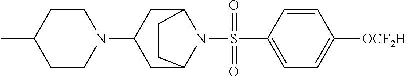

8-((4-(difluoromethoxy)phenyl)sulfonyl)-3-(4-methylpiperidin-1-yl)-8-azabicyclo[3.2.1]octane was prepared from 8-((4-(difluoromethoxy)phenyl)sulfonyl)-8-azabicyclo[3.2.1]octan-3-one (Intermediate 5) and 4-methylpiperidine using an analogous method to that described for Example 19. Purification by preparative HPLC (Waters SunFire C18 19*100 5 mkm column; gradient mixture water-ACN-FA 0.1% as a mobile phase) afforded the title compound (91 mg, 30%). LCMS m/z=415.2 [M+H]⁺.

### 2. Chiral Separation of 8-((4-(difluoromethoxy)phenyl)sulfonyl)-3-(4-methylpiperidin-1-yl)-8-azabicyclo[3.2.1]octane

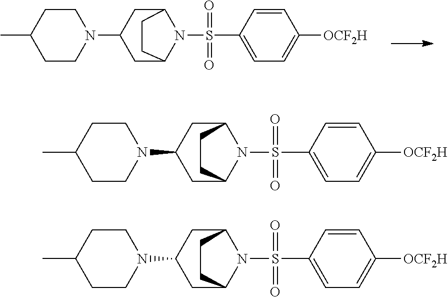

8-((4-(difluoromethoxy)phenyl)sulfonyl)-3-(4-methylpiperidin-1-yl)-8-azabicyclo[3.2.1]octane (60 mg) was separated into its stereoisomers using CHIRALPAK AD-H 4.6×150 mm, 5 μm, 30% MeOH w/0.1% DEA in CO₂ (flow rate: 100 mL/min, ABPR 120 bar, MBPR 40 psi, column temp 40° C.) to yield two stereoisomers:
Peak 1, Example 20, (1R,3r,5S)-8-((4-(difluoromethoxy)phenyl)sulfonyl)-3-(4-methylpiperidin-1-yl)-8-azabicyclo[3.2.1]octane (31 mg, t_{R}=2.01 min, 99.9% ee). ¹H NMR (400 MHZ, CD₃OD) δ (ppm): 8.01-7.91 (m, 2H), 7.32 (d, J=8.8 Hz, 2H), 7.26-6.75 (m, 1H), 4.31 (br s, 2H), 2.90 (br d, J=11.2 Hz, 2H), 2.76-2.61 (m, 1H), 2.12 (br t, J=11.6 Hz, 2H), 1.96-1.84 (m, 2H), 1.78-1.49 (m, 8H), 1.38 (br s, 1H), 1.27-1.12 (m, 2H), 0.94 (d, J=6.4 Hz, 3H). LCMS m/z=415.2 [M+H]⁺
Peak 2, Example 21, (1R,3s,5S)-8-((4-(difluoromethoxy)phenyl)sulfonyl)-3-(4-methylpiperidin-1-yl)-8-azabicyclo[3.2.1]octane (4 mg, t_{R}=2.32 min, 89.2% ee). ¹H NMR (400 MHZ, CD₃OD) δ (ppm): 7.99-7.83 (m, 2H), 7.32 (d, J=8.8 Hz, 2H), 7.25-6.80 (m, 1H), 4.25 (br s, 2H), 3.00 (br d, J=9.4 Hz, 2H), 2.47 (br s, 1H), 2.36-2.22 (m, 2H), 1.95 (br s, 2H), 1.74-1.51 (m, 8H), 1.44-1.29 (m, 1H), 1.25-1.12 (m, 2H), 0.93 (d, J=6.4 Hz, 3H). LCMS m/z=415.2 [M+H]⁺

### Example 22 and 23: 6-((1R,3s,5S)-8-((2-Methyl-6-(trifluoromethyl)pyridin-3-yl)sulfonyl)-8-azabicyclo[3.2.1]octan-3-yl)-2-oxa-6-azaspiro[3.3]heptane and 6-((1R,3r,5S)-8-((2-methyl-6-(trifluoromethyl)pyridin-3-yl)sulfonyl)-8-azabicyclo[3.2.1]octan-3-yl)-2-oxa-6-azaspiro[3.3]heptane

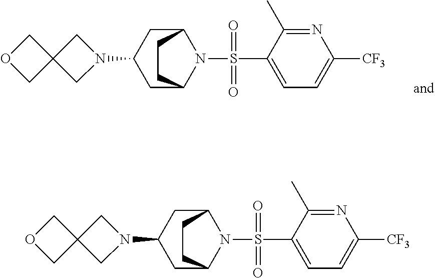 [SMILES: Cc1nc(C(F)(F)F)ccc1S(=O)(=O)N1[C@@H]2CC[C@H]1C[C@H](N1CC3(COC3)C1)C2 ; Cc1nc(C(F)(F)F)ccc1S(=O)(=O)N1[C@@H]2CC[C@H]1C[C@@H](N1CC3(COC3)C1)C2]

### *Stereochemistry Arbitrarily Assigned

6-((1R,5S)-8-((2-Methyl-6-(trifluoromethyl)pyridin-3-yl)sulfonyl)-8-azabicyclo[3.2.1]octan-3-yl)-2-oxa-6-azaspiro[3.3]heptane was prepared from 8-((2-methyl-6-(trifluoromethyl)pyridin-3-yl)sulfonyl)-8-azabicyclo[3.2.1]octan-3-one (Intermediate 6) and 2-oxa-6-azaspiro[3.3]heptane using an analogous method to that described for Example 19. Purification by column chromatography (24 g SiO₂, 20-50-100% EtOAc/EtOH (3/1) in heptane) afforded:
*Peak 1, Example 22; white solid (42 mg. 49%). ¹H NMR (400 MHZ, CD₃OD) δ (ppm): 8.50 (d, J=8.3 Hz, 1H), 7.81 (d, J=8.0 Hz, 1H), 4.69 (s, 4H), 4.09 (dd, J=4.4, 2.9 Hz, 2H), 3.21 (s, 4H), 2.88 (s, 3H), 2.35 (t. J=5.6 Hz, 1H), 2.16-2.05 (m, 2H), 1.89-1.74 (m, 4H), 1.69-1.60 (m, 2H). LCMS m/z=432.2 [M+H]⁺.
*Peak 2, Example 23; oil (6 mg, 6%). ¹H NMR (400 MHZ, CD₃OD) δ (ppm): 8.61-8.43 (m, 1H), 7.81 (d, J=8.0 Hz, 1H), 4.69 (s, 4H), 4.28-4.20 (m, 2H), 3.34 (s, 4H), 2.89 (s, 3H), 2.59-2.45 (m, 1H), 2.05-1.93 (m, 2H), 1.87-1.73 (m, 4H), 1.38-1.26 (m, 2H). LCMS m/z=432.1 [M+H]⁺.

### Example 24 and 25: (1R,3s,5S)-8-((2-Methyl-6-(trifluoromethyl)pyridin-3-yl)sulfonyl)-N-((tetrahydro-2H-pyran-4-yl)methyl)-8-azabicyclo[3.2.1]octan-3-amine and (1R,3r,5S)-8-((2-methyl-6-(trifluoromethyl)pyridin-3-yl)sulfonyl)-N-((tetrahydro-2H-pyran-4-yl)methyl)-8-azabicyclo[3.2.1]octan-3-amine

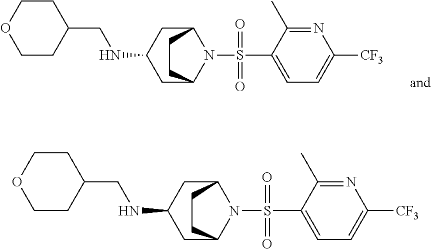 [SMILES: Cc1nc(C(F)(F)F)ccc1S(=O)(=O)N1[C@@H]2CC[C@H]1C[C@H](NCC1CCOCC1)C2 ; Cc1nc(C(F)(F)F)ccc1S(=O)(=O)N1[C@@H]2CC[C@H]1C[C@@H](NCC1CCOCC1)C2]

### *Stercochemistry Arbitrarily Assigned

(1R,5S)-8-((2-Methyl-6-(trifluoromethyl)pyridin-3-yl)sulfonyl)-N-((tetrahydro-2H-pyran-4-yl)methyl)-8-azabicyclo[3.2.1]octan-3-amine was prepared from 8-((2-methyl-6-(trifluoromethyl)pyridin-3-yl)sulfonyl)-8-azabicyclo[3.2.1]octan-3-one (Intermediate 6) and tetrahydropyran-4-ylmethanamine using an analogous method to that described for Example 19. Purification by column chromatography (24 g SiO₂, 20-100% EtOAc/EtOH (3/1) in heptane) afforded:
*Peak 1, Example 24: white solid (56 mg, 63%). ¹H NMR (400 MHZ, CD₃OD) δ (ppm): 8.57-8.46 (m, 1H), 7.82 (d, J=8.0 Hz, 1H), 4.15 (br d, J=2.8 Hz, 2H), 3.99-3.87 (m, 2H), 3.40 (td, J=11.7, 1.9 Hz, 2H), 2.96-2.84 (m, 4H), 2.43 (d, J=6.3 Hz, 2H), 2.28-2.19 (m, 2H), 2.10-1.96 (m, 2H), 1.94-1.82 (m, 2H), 1.79-1.55 (m, 5H), 1.37-1.17 (m, 2H). LCMS m/z=448.2 [M+H]⁺.
*Peak 2, Example 25: oil (5.5 mg, 6%). ¹H NMR (400 MHZ, CD₃OD) δ (ppm): 8.53 (d, J=8.3 Hz, 1H), 7.82 (d, J=8.0 Hz, 1H), 4.35-4.20 (m, 2H), 3.98-3.85 (m, 2H), 3.39 (td, J=11.8, 2.0 Hz, 2H), 2.92 (s, 4H), 2.45 (d, J=6.3 Hz, 2H), 2.07-1.93 (m, 4H), 1.81 (d, J=7.5 Hz, 2H), 1.70-1.61 (m, 3H), 1.48 (br s, 2H), 1.27-1.18 (m, 2H). LCMS m/z=448.1 [M+H]⁺.

### Example 26 and 27: (1R,3s,5S)-8-((2-Methyl-6-(trifluoromethyl)pyridin-3-yl)sulfonyl)-N-((3-methyloxetan-3-yl)methyl)-8-azabicyclo[3.2.1]octan-3-amine and (1R,3r,5S)-8-((2-methyl-6-(trifluoromethyl)pyridin-3-yl)sulfonyl)-N-((3-methyloxetan-3-yl)methyl)-8-azabicyclo[3.2.1]octan-3-amine

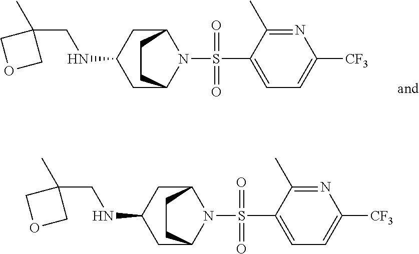 [SMILES: Cc1nc(C(F)(F)F)ccc1S(=O)(=O)N1[C@@H]2CC[C@H]1C[C@H](NCC1(C)COC1)C2 ; Cc1nc(C(F)(F)F)ccc1S(=O)(=O)N1[C@@H]2CC[C@H]1C[C@@H](NCC1(C)COC1)C2]

### *Stereochemistry Arbitrarily Assigned

(1R,5S)-8-((2-Methyl-6-(trifluoromethyl)pyridin-3-yl)sulfonyl)-N-((3-methyloxetan-3-yl)methyl)-8-azabicyclo[3.2.1]octan-3-amine was prepared from 8-((2-methyl-6-(trifluoromethyl)pyridin-3-yl)sulfonyl)-8-azabicyclo[3.2.1]octan-3-one (Intermediate 6) and (3-methyloxetan-3-yl) methanamine using an analogous method to that described for Example 19. Purification by column chromatography (24 g SiO₂, 20-100% EtOAc/EtOH (3/1) in heptane) afforded:
*Peak 1. Example 26: white solid (46 mg, 53%). ¹H NMR (400 MHZ, CD₃OD) δ (ppm): 8.52 (d, J=8.0 Hz, 1H), 7.82 (d, J=8.3 Hz, 1H), 4.48 (d, J=5.8 Hz, 2H), 4.34 (d, J=5.5 Hz, 2H), 4.20-4.10 (m, 2H), 2.90 (s, 4H), 2.72 (s, 2H), 2.30-2.19 (m, 2H), 2.08-1.96 (m, 2H), 1.93-1.74 (m, 4H), 1.31 (s, 3H). LCMS m/z=434.2 [M+H]⁺.
*Peak 2, Example 27: white solid (6 mg, 7%). ¹H NMR (400 MHZ, CD₃OD) δ (ppm): 8.57-8.49 (m, 1H), 7.82 (d, J=8.0 Hz, 1H), 4.42 (d, J=5.8 Hz, 2H), 4.32 (d, J=5.8 Hz, 2H), 4.27 (br dd, J=4.1, 2.9 Hz, 2H), 3.00-2.88 (m, 4H), 2.77 (s, 2H), 2.06-1.95 (m, 4H), 1.86-1.78 (m, 2H), 1.56-1.43 (m, 2H), 1.28 (s, 3H). LCMS m/z=434.2 [M+H]⁺.

### Example 28: (1R,3s,5S)-8-((4-(Difluoromethoxy)phenyl)sulfonyl)-N-((3-methyloxetan-3-yl)methyl)-8-azabicyclo[3.2.1]octan-3-amine

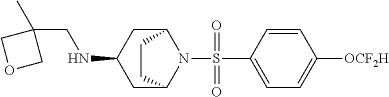

To a mixture of (1R,3s,5S)-8-((4-(difluoromethoxy)phenyl)sulfonyl)-8-azabicyclo[3.2.1]octan-3-amine (Intermediate 4, 45 mg. 0.1 mmol) and 3-methyloxetane-3-carbaldehyde (14 mg. 0.1 mmol) in DCM (3 mL) was added acetic acid (17 μL, 0.3 mmol). NaBH(OAc)₃ (85 mg, 0.4 mmol) was added in one portion and the mixture stirred at room temperature overnight. The reaction was quenched with sat. aq. NH₄Cl, and the mixture was stirred at room temperature for 5 min. Additional DCM and sat. NaHCO₃were added, and the reaction mixture stirred for another 5 min. The organic phase was washed with water then evaporated to dryness in vacuo. The residue was dissolved in DMSO purified by prep-HPLC (Waters Xselect CSH Prep C18 30×100 mm, 5 μm; 5-55% MeCN in water (+NH₄OH)) to afford (1R,3s,5S)-8-((4-(difluoromethoxy)phenyl)sulfonyl)-N-((3-methyloxetan-3-yl)methyl)-8-azabicyclo[3.2.1]octan-3-amine as a white solid (27 mg, 64%). LCMS m/z=417.1 [M+H]⁺, t_{R}=0.57 min.

### Example 29: (1R,3s,5S)-8-((4-(Difluoromethoxy)phenyl)sulfonyl)-N-((tetrahydro-2H-pyran-4-yl)methyl)-8-azabicyclo[3.2.1]octan-3-amine

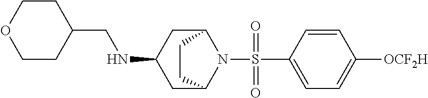

Acetic acid (21 μL, 0.4 mmol) and NaBH(OAc)₃ (105 mg, 0.5 mmol) were added to a mixture of (1R,3s,5S)-8-((4-(difluoromethoxy)phenyl)sulfonyl)-8-azabicyclo[3.2.1]octan-3-amine trifluoroacetate (Intermediate 4, 55 mg, 0.1 mmol) and tetrahydropyran-4-carbaldehyde (15.5 mg, 0.1 mmol) in DCM (2 mL), and the mixture stirred at room temperature for 2 h. The reaction was quenched with sat. aq. NaHCO₃, diluted with DCM and stirred for 5 min. The organic phase was washed with water, dried (MgSO₄) and evaporated to dryness in vacuo. The residue was purified by column chromatography (12 g SiO₂, EtOAc/EtOH 3/1) to afford (1R,3s,5S)-8-((4-(difluoromethoxy)phenyl)sulfonyl)-N-((tetrahydro-2H-pyran-4-yl)methyl)-8-azabicyclo[3.2.1]octan-3-amine as a white solid (32 mg, 60%). ¹H NMR (400 MHZ, CD₃OD) δ (ppm): 8.01-7.89 (m, 2H), 7.32 (d, J=8.5 Hz, 2H), 7.23-6.78 (m, 1H), 4.29 (br s, 2H), 3.94 (br dd, J=11.4, 4.1 Hz, 2H), 3.42 (t, J=11.7 Hz, 2H), 2.91 (dt, J=11.2, 5.6 Hz, 1H), 2.46 (d, J=6.5 Hz, 2H), 2.09-1.94 (m, 2H), 1.79-1.45 (m, 9H), 1.38-1.15 (m, 2H). LCMS m/z=431.2 [M+H]⁺.

### Example 30: 8-((4-(Difluoromethoxy)phenyl)sulfonyl)-N-(1-methyl-2-oxabicyclo[2.1.1]hexan-4-yl)-8-azabicyclo[3.2.1]octan-3-amine

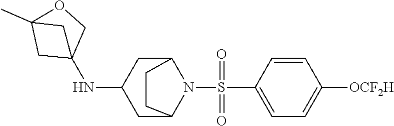

DIPEA (70 μL, 0.4 mmol) was added to a mixture of 1-methyl-2-oxabicyclo[2.1.1]hexan-4-amine HCl salt (36 mg, 0.2 mmol) and 8-[4-(difluoromethoxy)phenyl]sulfonyl-8-azabicyclo[3.2.1]octan-3-one (66 mg, 0.2 mmol) in DCM (3 mL). NaBH(OAc)₃ (170 mg, 0.8 mmol) was added in one portion to the above solution, and the mixture was stirred at room temperature overnight. Additional NaBH(OAc)₃ (85 mg, 0.4 mmol) was added and stirring continued at room temperature overnight. The reaction was quenched with sat. aq. NH₄Cl and stirred at room temperature for 5 min. The reaction mixture was diluted with DCM and sat. aq. NaHCO₃ and stirred for another 5 min. The organic phase was washed with water and concentrated. The residue was dissolved in DMSO and purified by prep-HPLC (Waters Xselect CSH Prep C18 30×100 mm, 5 μm; 5-70% MeCN in water (+NH₄OH)) to afford 8-((4-(difluoromethoxy)phenyl)sulfonyl)-N-(1-methyl-2-oxabicyclo[2.1.1]hexan-4-yl)-8-azabicyclo[3.2.1]octan-3-amine as a white solid (20 mg, 23%). LCMS m/z=429.1 [M+H]⁺, t_{R}=0.60 min.

### Example 31: (1R,3s,5S)-8-((4-(Difluoromethoxy)phenyl)sulfonyl)-N-(oxetan-3-ylmethyl)-8-azabicyclo[3.2.1]octan-3-amine

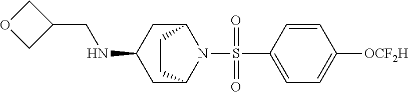

(1R,3s,5S)-8-((4-(difluoromethoxy)phenyl)sulfonyl)-N-(oxetan-3-ylmethyl)-8-azabicyclo[3.2.1]octan-3-amine was prepared as a white solid (23.8 mg, 59%) from (1R,3s,5S)-8-((4-(difluoromethoxy)phenyl)sulfonyl)-8-azabicyclo[3.2.1]octan-3-amine (Intermediate 4) and oxetane-3-carbaldehyde using an analogous method to that described for Example 28 to afford the desired compound (31 mg, 76%). LCMS m/z=402.9 [M+H]⁺, t_{R}=0.56 min.

### Example 32: (1R,3r,5S)-8-((4-(Difluoromethoxy)phenyl)sulfonyl)-N-((tetrahydro-2H-pyran-4-yl)methyl)-8-azabicyclo[3.2.1]octan-3-amine

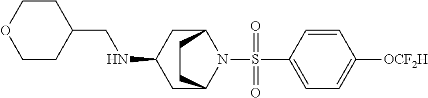

(1R,3r,5S)-8-((4-(difluoromethoxy)phenyl)sulfonyl)-N-((tetrahydro-2H-pyran-4-yl)methyl)-8-azabicyclo[3.2.1]octan-3-amine (35 mg, 75%) was prepared from (1R,3r,5S)-8-((4-(difluoromethoxy)phenyl)sulfonyl)-8-azabicyclo[3.2.1]octan-3-amine (Intermediate 3) and tetrahydro-2H-pyran-4-carbaldehyde using an analogous method to that described for Example 28. LCMS m/z=431.2 [M+H]⁺, t_{R}=0.59 min.

### Example 33: (1R,3r,5S)-8-((4-(Difluoromethoxy)phenyl)sulfonyl)-3-(morpholinomethyl)-8-azabicyclo[3.2.1]octan-3-ol

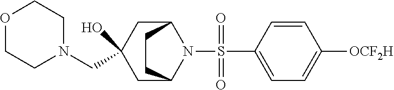

### 1. Synthesis of tert-butyl (1R,3r,5S)-8-azaspiro [bicyclo[3.2.1]octane-3,2′-oxirane]-8-carboxylate

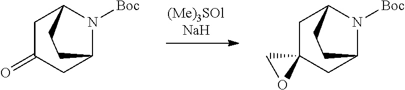 [SMILES: CC(C)(C)OC(=O)N1[C@@H]2CC[C@H]1CC(=O)C2 ; CC(C)(C)OC(=O)N1[C@@H]2CC[C@H]1C[C@]1(CO1)C2]

Trimethylsulfoxonium iodide (2.93 g, 13.32 mmol) was added to a suspension of NaH (533 mg, 13.3 mmol, 60% purity) in DMSO (5 mL) and stirred under N₂ at 25° C. for 1 h before a solution of tert-butyl (1R,5S)-3-oxo-8-azabicyclo[3.2.1]octane-8-carboxylate (2 g, 8.9 mmol) in DMSO (5 mL) was added. After consumption of starting material, water (20 mL) was added, and the resulting mixture extracted with DCM (3×50 mL). The combined organics were washed with brine (20 mL), dried (Na₂SO₄), filtered, and evaporated to dryness in vacuo. The residue was purified by column chromatography (SiO₂, 0-17% EtOAc/PE) to give compound tert-butyl (1R,3r,5S)-8-azaspiro[bicyclo[3.2.1]octane-3,2′-oxirane]-8-carboxylate as a colorless oil (1.5 g, 71%). LCMS m/z=183.9 [M-^{t-}Bu+H]⁺.

### 2. Synthesis of tert-butyl (1R,3r,5S)-3-hydroxy-3-(morpholinomethyl)-8-azabicyclo[3.2.1]octane-8-carboxylate

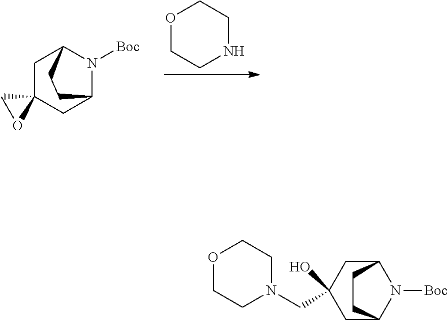 [SMILES: CC(C)(C)OC(=O)N1[C@@H]2CC[C@H]1C[C@]1(CO1)C2 ; C1COCCN1 ; CC(C)(C)OC(=O)N1[C@@H]2CC[C@H]1C[C@](O)(CN1CCOCC1)C2]

Morpholine (145 μL, 1.7 mmol) was added to a solution of tert-butyl (1R,3r,5S)-8-azaspiro [bicyclo[3.2.1]octane-3,2′-oxirane]-8-carboxylate (200 mg, 0.8 mmol) in EtOH (5 mL) and the resulting mixture stirred under N₂ at 25° C. for 1 h and then at 100° C. for an additional 15 h. The reaction mixture was evaporated to dryness in vacuo and the residue purified by column chromatography (ISCO®; 12 g SepaFlash® Silica Flash Column, 30-90% EtOAc/PE) to afford tert-butyl (1R,3r,5S)-3-hydroxy-3-(morpholinomethyl)-8-azabicyclo[3.2.1]octane-8-carboxylate as a colorless oil (130 mg, 48%). LCMS m/z=327.1 [M+H]⁺.

### 3. Synthesis of (1R,3r,5S)-3-(morpholinomethyl)-8-azabicyclo[3.2.1]octan-3-ol trifluoroacetate

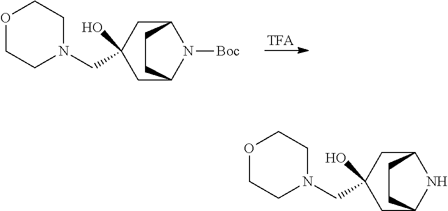 [SMILES: CC(C)(C)OC(=O)N1[C@@H]2CC[C@H]1C[C@](O)(CN1CCOCC1)C2 ; O[C@@]1(CN2CCOCC2)C[C@H]2CC[C@@H](C1)N2]

TFA (2.98 g, 26.1 mmol, 2.00 mL) was added to a solution of compound tert-butyl (1R,3r,5S)-3-hydroxy-3-(morpholinomethyl)-8-azabicyclo[3.2.1]octane-8-carboxylate (130 mg, 0.4 mmol) in DCM (10 mL) and the mixture was stirred under N₂ at 25° C. for 0.5 h. The reaction mixture was evaporated to dryness in vacuo to give (1R,3r,5S)-3-(morpholinomethyl)-8-azabicyclo[3.2.1]octan-3-ol trifluoroacetate as a yellow oil (150 mg, crude) which was used without further purification. ¹H NMR (400 MHZ, CD₃OD) δ (ppm): 3.58 (br s, 2H), 3.18 (s, 4H), 2.54-2.46 (m, 4H), 2.16-2.13 (m, 6H), 2.11-2.06 (m, 4H).

### 4. Synthesis of (1R,3r,5S)-8-((4-(difluoromethoxy)phenyl)sulfonyl)-3-(morpholinomethyl)-8-azabicyclo[3.2.1]octan-3-ol

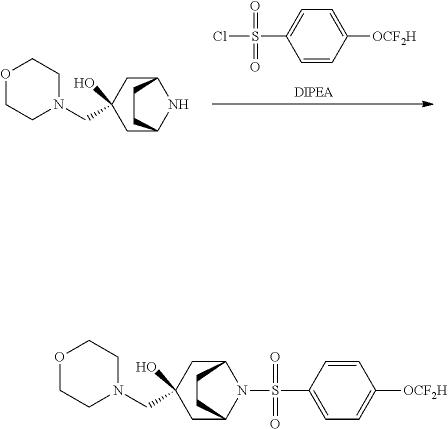

4-(Difluoromethoxy) benzenesulfonyl chloride (100 mg, 0.4 mmol) was added to a solution of (1R,3r,5S)-3-(morpholinomethyl)-8-azabicyclo[3.2.1]octan-3-ol trifluoroacetate (100 mg, 0.4 mmol) and DIPEA (340 μL, 1.9 mmol) in DCM (20 mL) at 0° C. and the resulting mixture stirred under N₂ at 25° C. for 1 h. Water (10 mL) was added to the reaction mixture and extracted with DCM (3×30 mL). The combined organics were washed with brine (20 mL), dried (Na₂SO₄), filtered, and evaporated to dryness in vacuo. The residue was purified by prep-HPLC (Welch Xtimate C18 25×150 mm, 5 mm; 50-80% MeCN/water (10 mM NH₄HCO₃)) to give (1R,3r,5S)-8-((4-(difluoromethoxy)phenyl)sulfonyl)-3-(morpholinomethyl)-8-azabicyclo[3.2.1]octan-3-ol as a white solid (80 mg, 49%). ¹H NMR (400 MHZ, CD₃OD) δ (ppm): 7.92 (d, J=8.8 Hz, 2H), 7.29 (d, J=8.4 Hz, 2H), 7.00 (t, J=73.2 Hz, 1H), 4.21 (br s, 2H), 3.67-3.63 (m, 4H), 2.55-2.52 (m, 4H), 2.14-2.08 (m, 4H), 1.89-1.84 (m, 2H), 1.76-1.72 (m, 2H), 1.42-1.40 (m, 2H). LCMS m/z (ESI): 433.1 [M+H]⁺.

### Example 34: (1R,3s,5S)-8-((4-(Difluoromethoxy)phenyl)sulfonyl)-3-(morpholinomethyl)-8-azabicyclo[3.2.1]octan-3-ol

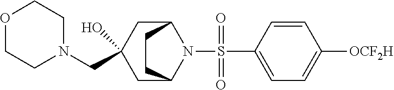

### 1. Synthesis of tert-butyl (1R,5S)-3-methylene-8-azabicyclo[3.2.1]octane-8-carboxylate

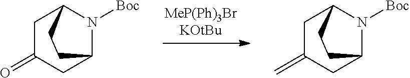 [SMILES: CC(C)(C)OC(=O)N1[C@@H]2CC[C@H]1CC(=O)C2 ; C=C1C[C@H]2CC[C@@H](C1)N2C(=O)OC(C)(C)C]

Methyl(triphenyl)phosphonium bromide (55.50 g, 155.4 mmol) was added to a solution of potassium tert-butoxide (1 M, 155.4 mL) in THF (40 mL) under N₂ at 0° C. and the resulting mixture stirred at 0° C. for 15 min followed by heating at 80° C. for 1 h. The reaction mixture was cooled to room temperature and a solution of tert-butyl (1S,5R)-3-oxo-8-azabicyclo[3.2.1]octane-8-carboxylate (7 g, 31.1 mmol) in THF (60 mL) was added dropwise and stirred at 25° C. for 15 h. The reaction mixture was diluted with acetone (30 mL) and the resulting solids removed by filtration. The filtrate was diluted with water (80 mL) and extracted with EtOAc (3×100 mL). The combined organics were washed with brine (50 mL), dried (Na₂SO₄), filtered, and evaporated to dryness in vacuo. The residue was purified by column chromatography (ISCO®; 330 g SepaFlash® Silica Flash Column, Eluent of 5-20% EtOAc/PE gradient) to give tert-butyl (1R,5S)-3-methylene-8-azabicyclo[3.2.1]octane-8-carboxylate as a colorless oil (5 g, 72%). LCMS m/z (ESI): 168.1 [M-^{t-}Bu+H]⁺.

### 2. Synthesis of tert-butyl (1R,3r,5S)-8-azaspiro [bicyclo[3.2.1]octane-3,2′-oxirane]-8-carboxylate and tert-butyl (1R,3s,5S)-8-azaspiro [bicyclo[3.2.1]octane-3,2′-oxirane]-8-carboxylate and

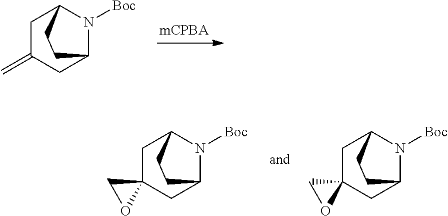 [SMILES: C=C1C[C@H]2CC[C@@H](C1)N2C(=O)OC(C)(C)C ; CC(C)(C)OC(=O)N1[C@@H]2CC[C@H]1C[C@@]1(CO1)C2 ; CC(C)(C)OC(=O)N1[C@@H]2CC[C@H]1C[C@]1(CO1)C2]

3-Chlorobenzenecarboperoxoic acid (22.7 g, 112 mmol, 85% purity) was added to a solution of tert-butyl (1R,5S)-3-methylene-8-azabicyclo[3.2.1]octane-8-carboxylate (10 g, 44.8 mmol) in DCM (100 mL) at 0° C. The reaction mixture was stirred at 25° C. for 24 h before additional 3-chlorobenzenecarboperoxoic acid (9.09 g, 44.8 mmol, 85% purity) was added and stirring continued at 25° C. for another 24 h. The resulting solid were removed by filtration and sat. aq. NaHCO₃ (50 mL) was added to the filtrate. The aqueous phase was extracted with DCM (3×80 mL). The combined organics were washed with sat. aq. Na₂SO₃ (50 mL), brine (20 mL), dried (Na₂SO₄), filtered, and evaporated to dryness in vacuo. The residue was purified by column chromatography (ISCO®; 120 g SepaFlash® Silica Flash Column, 15-35% EtOAc/PE) to give:
Peak 1: tert-Butyl (1R,3s,5S)-8-azaspiro [bicyclo[3.2.1]octane-3,2′-oxirane]-8-carboxylate as a white solid (2.5 g, 23%). ¹H NMR (400 MHZ, CDCl₃) δ (ppm): 4.29 (br s, 2H), 2.72 (s, 2H), 2.32-2.11 (m, 2H), 2.02-1.92 (m, 2H), 1.72 (br d, J=7.6 Hz, 2H), 1.50-1.42 (m, 9H), 1.25 (br d, J=7.2 Hz, 2H). LCMS m/z (ESI): 140.1 [M-Boc+H]⁺.
Peak 2: tert-Butyl (1R,3r,5S)-8-azaspiro [bicyclo[3.2.1]octane-3,2′-oxirane]-8-carboxylate (4.5 g, 42%). ¹H NMR (400 MHZ, CDCl₃) δ (ppm): 4.42-4.19 (m, 2H), 2.48-2.19 (m, 4H), 2.10-1.96 (m, 4H), 1.46 (s, 9H), 1.17 (br d, J=14.0 Hz, 2H).

### 3. Synthesis of tert-butyl (1R,3s,5S)-3-(aminomethyl)-3-hydroxy-8-azabicyclo[3.2.1]octane-8-carboxylate

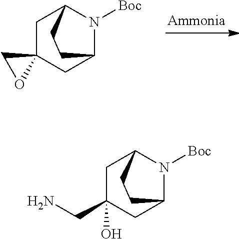 [SMILES: CC(C)(C)OC(=O)N1[C@@H]2CC[C@H]1C[C@@]1(CO1)C2 ; CC(C)(C)OC(=O)N1[C@@H]2CC[C@H]1C[C@@](O)(CN)C2]

A mixture of tert-butyl (1R,3s,5S)-8-azaspiro [bicyclo[3.2.1]octane-3,2′-oxirane]-8-carboxylate (1 g, 4.2 mmol) and ammonia (7 M, 13.3 mL) in water was heated in an autoclave at 60° C. for 96 h. The mixture was concentrated under reduced pressure to give tert-butyl (1R,3s,5S)-3-(aminomethyl)-3-hydroxy-8-azabicyclo[3.2.1]octane-8-carboxylate as a yellow oil (1 g, crude) which was used without further purification. ¹H NMR (400 MHZ, CD₃OD) δ (ppm): 4.35-4.21 (m, 2H), 2.76 (s, 2H), 2.10-1.93 (m, 4H), 1.81 (br d, J=7.2 Hz, 2H), 1.47 (s, 9H), 1.29 (br d, J=12.4 Hz, 2H). LCMS m/z (ESI): 201.1 [M-^{t}Bu+H]⁺.

### 4. Synthesis of tert-butyl (1R,3s,5S)-3-hydroxy-3-(morpholinomethyl)-8-azabicyclo[3.2.1]octane-8-carboxylate

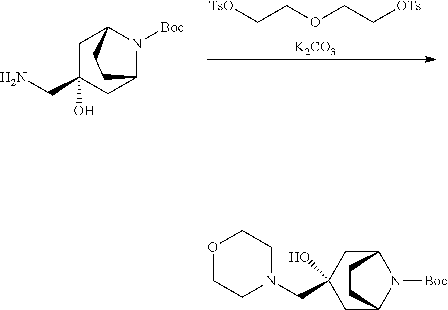

Oxybis(ethane-2,1-diyl) bis(4-methylbenzenesulfonate) (2.91 g, 7.0 mmol) was added to a mixture of tert-butyl (1R,3s,5S)-3-(aminomethyl)-3-hydroxy-8-azabicyclo[3.2.1]octane-8-carboxylate (1.2 g, 4.7 mmol) and K₂CO₃ (1.94 g, 14.0 mmol) in MeCN (30 mL) under N₂ and the mixture stirred at 80° C. for 15 h. The solids were removed by filtration and the filtrate concentrated under reduced pressure. The residue was purified by column chromatography (ISCO®; 20 g SepaFlash® Silica Flash Column; 0-10% MeOH/DCM) followed by prep-HPLC (YMC-Actus Triart C18 150×30 mm, 5 μm, 4-60% MeCN/water (0.05% NH₄OH)) to afford tert-butyl (1R,3s,5S)-3-hydroxy-3-(morpholinomethyl)-8-azabicyclo[3.2.1]octane-8-carboxylate as a white solid (22 mg, 1.44%). ¹H NMR (400 MHZ, DMSO-d₆) δ (ppm): 4.27 (s, 1H), 4.00 (br s, 2H), 3.56 (br t, J=4.0 Hz, 4H), 3.44-3.38 (m, 4H), 2.36 (s, 2H), 1.96 (br s, 2H), 1.80 (br s, 2H), 1.71-1.54 (m, 4H), 1.41 (s, 9H). LCMS m/z (ESI): 327.2 [M+H]⁺.

### 5. Synthesis of (1R,3s,5S)-3-(morpholinomethyl)-8-azabicyclo[3.2.1]octan-3-ol hydrochloride

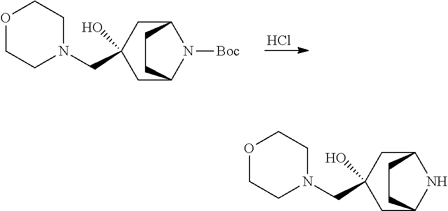 [SMILES: CC(C)(C)OC(=O)N1[C@@H]2CC[C@H]1C[C@@](O)(CN1CCOCC1)C2 ; Cl ; O[C@]1(CN2CCOCC2)C[C@H]2CC[C@@H](C1)N2]

HCl/dioxane (4 M, 2 mL) was added to a solution of tert-butyl (1R,3s,5S)-3-hydroxy-3-(morpholinomethyl)-8-azabicyclo[3.2.1]octane-8-carboxylate (25 mg, 0.1 mmol) in MeOH (2 mL) and the resulting mixture stirred at 20° C. for 12 h. The reaction mixture was concentrated under reduced pressure to give (1R,3s,5S)-3-(morpholinomethyl)-8-azabicyclo[3.2.1]octan-3-ol hydrochloride as a yellow oil (35 mg) which was used without further purification. LCMS m/z (ESI): 227.1 [M+H]⁺.

### 6. Synthesis of (1R,3s,5S)-8-((4-(difluoromethoxy)phenyl)sulfonyl)-3-(morpholinomethyl)-8-azabicyclo[3.2.1]octan-3-ol

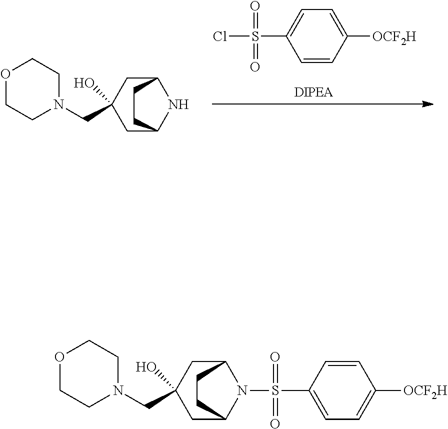

To a solution of compound (1R,3s,5S)-3-(morpholinomethyl)-8-azabicyclo[3.2.1]octan-3-ol hydrochloride (30 mg, 0.1 mmol) in DCM (3 mL) was added DIPEA (60 μL, 0.3 mmol,) and 4-(difluoromethoxy) benzenesulfonyl chloride (41.6 mg, 0.2 mmol) at 0° C. The mixture was stirred at 20° C. for 3 h and quenched with water (10 mL). The reaction mixture was extracted with DCM (3×10 mL) and the combined organics washed with brine (3×10 mL), dried (Na₂SO₄), filtered, and evaporated to dryness in vacuo. The residue was purified by prep-HPLC (Welch Xtimate C18 25×150 mm, 5 μm; 33-63% MeCN/water (10 mM NH₄HCO₃)) to give (1R,3s,5S)-8-((4-(difluoromethoxy)phenyl)sulfonyl)-3-(morpholinomethyl)-8-azabicyclo[3.2.1]octan-3-ol as a white solid (14.5 mg, 29%). ¹H NMR (400 MHZ, DMSO-d₆) δ (ppm): 7.90 (d, J=8.8 Hz, 2H), 7.39-7.08 (m, 3H), 4.40 (s, 1H), 4.13 (br s, 2H), 3.50 (d, J=4.2 Hz, 2H), 2.43-2.40 (m, 6H), 2.22 (s, 2H), 2.07 (d, J=14.4 Hz, 2H), 1.72 (dd, J=4.0, 13.2 Hz, 2H), 1.44 (d, J=8.0 Hz, 2H), 1.26-1.12 (m, 2H). LCMS m/z (ESI): 433.1 [M+H]⁺.

### Example 35: 4-(((1R,3s,5S)-8-((4-(Difluoromethoxy)-2,6-difluorophenyl)sulfonyl)-8-azabicyclo[3.2.1]octan-3-yl)methyl)morpholine

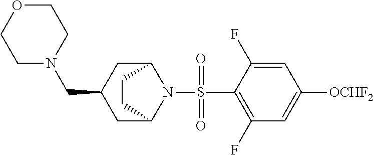 [SMILES: O=S(=O)(c1c(F)cc(OC(F)F)cc1F)N1[C@@H]2CC[C@H]1C[C@H](CN1CCOCC1)C2]

DIPEA (70 μL, 0.4 mmol) was added to a mixture of 4-(((1R,3s,5S)-8-azabicyclo[3.2.1]octan-3-yl)methyl)morpholine dihydrochloride (Intermediate 7, 31 mg, 0.1 mmol) and 4-(difluoromethoxy)-2,6-difluoro-benzenesulfonyl chloride (28 mg, 0.1 mmol) in DCM (2 mL) and the mixture stirred at room temperature for 2 h. The reaction was quenched with sat. aq. NaHCO₃diluted with water and stirred at room temperature for 5 min. The organic phase was washed with water and evaporated to dryness in vacuo. The residue was triturated with small amount of MeCN to afford 4-(((1R,3s,5S)-8-((4-(difluoromethoxy)-2,6-difluorophenyl)sulfonyl)-8-azabicyclo[3.2.1]octan-3-yl)methyl)morpholine as a white solid (22 mg, 48%). ¹H NMR (400 MHZ, CD₃OD) δ (ppm): 7.33-6.81 (m, 3H), 4.33 (br d, J=2.3 Hz, 2H), 3.79-3.59 (m, 4H), 2.50-2.31 (m, 4H), 2.20-2.03 (m, 3H), 1.82-1.66 (m, 6H), 1.45-1.34 (m, 2H). LCMS m/z=453.1 [M+H]⁺.

### Example 36: 4-(((1R,3s,5S)-8-((1,3-Dimethyl-1H-pyrazol-5-yl)sulfonyl)-8-azabicyclo[3.2.1]octan-3-yl)methyl)morpholine

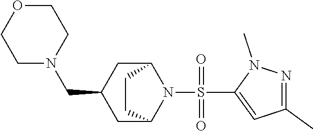 [SMILES: Cc1cc(S(=O)(=O)N2[C@@H]3CC[C@H]2C[C@H](CN2CCOCC2)C3)n(C)n1]

4-(((1R,3s,5S)-8-((1,3-Dimethyl-1H-pyrazol-5-yl)sulfonyl)-8-azabicyclo[3.2.1]octan-3-yl)methyl)morpholine was prepared as a white solid (43 mg, 90%) from 4-(((1R,3s,5S)-8-azabicyclo[3.2.1]octan-3-yl)methyl)morpholine dihydrochloride (Intermediate 7) and 2,5-dimethylpyrazole-3-sulfonyl chloride using an analogous method to that described for Example 35. Purified by column chromatography (12 g, SiO₂, 7:1 EtOAc/EtOH). ¹H NMR (400 MHZ, CD₃OD) δ (ppm): 6.62 (s, 1H), 4.36-4.18 (m, 2H), 4.00 (s, 3H), 3.77-3.62 (m, 4H), 2.51-2.32 (m, 4H), 2.25 (s, 3H), 2.21-2.15 (m, 2H), 2.15-2.04 (m, 1H), 1.83-1.70 (m, 6H), 1.45-1.34 (m, 2H). LCMS m/z=369.2 [M+H]⁺.

### Example 37: 4-(((1R,3r,5S)-8-((1,3-Dimethyl-1H-pyrazol-5-yl)sulfonyl)-8-azabicyclo[3.2.1]octan-3-yl)methyl)morpholine

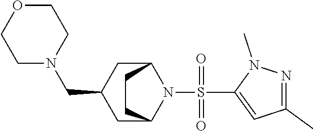 [SMILES: Cc1cc(S(=O)(=O)N2[C@@H]3CC[C@H]2C[C@@H](CN2CCOCC2)C3)n(C)n1]

4-(((1R,3r,5S)-8-((1,3-dimethyl-1H-pyrazol-5-yl)sulfonyl)-8-azabicyclo[3.2.1]octan-3-yl)methyl)morpholine was prepared as a colorless oil (29 mg, 70%) from 4-(((1R,3r,5S)-8-azabicyclo[3.2.1]octan-3-yl)methyl)morpholine dihydrochloride (Intermediate 8) and 2,5-dimethylpyrazole-3-sulfonyl chloride using an analogous method to that described for Example 35. ¹H NMR (400 MHZ, CD₃OD) δ (ppm): 6.60 (s, 1H), 4.30-4.17 (m, 2H), 3.97 (s, 3H), 3.70-3.62 (m, 4H), 2.40 (br d, J=4.5 Hz, 4H), 2.23 (s, 3H), 2.19-2.04 (m, 3H), 1.82-1.67 (m, 6H), 1.44-1.31 (m, 2H). LCMS m/z=369.2 [M+H]⁺.

### Example 38: 4-(((1R,3s,5S)-8-((3,5-Dimethylisoxazol-4-yl)sulfonyl)-8-azabicyclo[3.2.1]octan-3-yl)methyl)morpholine

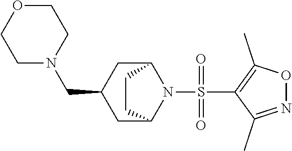 [SMILES: Cc1noc(C)c1S(=O)(=O)N1[C@@H]2CC[C@H]1C[C@H](CN1CCOCC1)C2]

4-(((1R,3r,5S)-8-((1,3-Dimethyl-1H-pyrazol-5-yl)sulfonyl)-8-azabicyclo[3.2.1]octan-3-yl)methyl)morpholine was prepared as a white solid (46 mg, 83%) from 4-(((1R,3s,5S)-8-azabicyclo[3.2.1]octan-3-yl)methyl)morpholine dihydrochloride (Intermediate 7) and 3,5-dimethylisoxazole-4-sulfonyl chloride using an analogous method to that described for Example 35. ¹H NMR (400 MHZ, CD₃OD) δ (ppm): 4.26-4.13 (m, 2H), 3.76-3.59 (m, 4H), 2.63 (s, 3H), 2.48-2.28 (m, 7H), 2.18-2.14 (m, 2H), 2.12-1.98 (m, 1H), 1.91-1.63 (m, 6H), 1.35 (s, 2H). LCMS m/z=370.2 [M+H]⁺.

### Example 39: 4-(((1R,3s,5S)-8-((4,6-Dimethylpyridin-3-yl)sulfonyl)-8-azabicyclo[3.2.1]octan-3-yl)methyl)morpholine

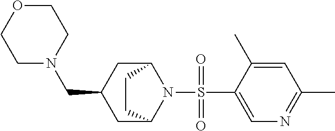 [SMILES: Cc1cc(C)c(S(=O)(=O)N2[C@@H]3CC[C@H]2C[C@H](CN2CCOCC2)C3)cn1]

4-(((1R,3s,5S)-8-((4,6-Dimethylpyridin-3-yl)sulfonyl)-8-azabicyclo[3.2.1]octan-3-yl)methyl)morpholine was prepared as a white solid (38 mg, 50%) from 4-(((1R,3s,5S)-8-azabicyclo[3.2.1]octan-3-yl)methyl)morpholine dihydrochloride (Intermediate 7) and 4,6-dimethylpyridine-3-sulfonyl chloride using an analogous method to that described for Example 35. ¹H NMR (400 MHZ, CD₃OD) δ (ppm): 8.82 (s, 1H), 7.34 (s, 1H), 4.17 (br s, 2H), 3.71-3.60 (m, 4H), 2.64 (s, 3H), 2.57 (s, 3H), 2.38 (br s, 4H), 2.17-2.12 (m, 2H), 2.12-2.04 (m, 1H), 1.97-1.90 (m, 2H), 1.80-1.68 (m, 4H), 1.32 (br t, J=10.9 Hz, 2H). LCMS m/z=380.2 [M+H]⁺.

### Example 59: rac-4-(((1R,3r,5S)-8-((4-(difluoromethoxy)phenyl)sulfonyl)-8-azabicyclo[3.2.1]octan-3-yl)amino)butan-2-ol

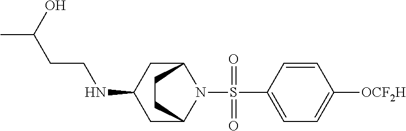

### 1. Synthesis of tert-butyl (1R,3r,5S)-3-(3-hydroxybutanamido)-8-azabicyclo[3.2.1]octane-8-carboxylate

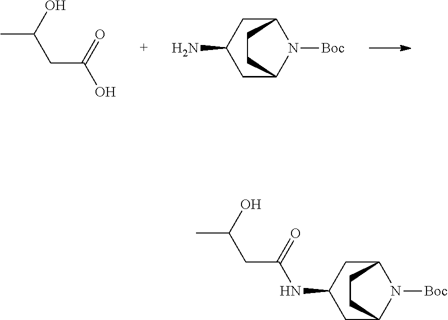

3-Hydroxybutanoic acid (193.20 mg, 1.86 mmol), tert-butyl (1R,3r,5S)-3-amino-8-azabicyclo[3.2.1]octane-8-carboxylate (400 mg, 1.77 mmol) and NMI (507.90 mg, 6.19 mmol, 493.11 μL) were combined and dissolved in MeCN (15 mL). TCFH (743.87 mg, 2.65 mmol) was added in a single portion and the reaction was monitored by LCMS. After 12 h the mixture was diluted with water (15 mL) and extracted with EtOAc (20 mL×3). The combined organic phase was washed with brine (30 mL×3), dried over anhydrous sodium sulfate, filtered and concentrated in vacuum. The crude mixture was dissolved in MeOH (10 mL) and K₂CO₃ (488.54 mg, 3.53 mmol) was added. The resulting mixture was stirred for 1 h at 22° C. and then diluted with water (15 mL) and extracted with EtOAc (20 mL×3). The combined organic phase was washed with brine (30 mL×3), dried over anhydrous sodium sulfate, filtered and concentrated in vacuo. The crude material was purified by flash column (MeOH in DCM=0%˜1%) to give the desired compound (380 mg, 69% yield) as a white solid. ¹H NMR (400 MHZ, CDCl₃) δ ppm 6.67 (d, J=7.2 Hz, 1H), 4.21-4.13 (m, 4H), 2.24-2.15 (m, 2H), 2.05-2.00 (m, 4H), 1.85-1.80 (m, 2H), 1.70-1.61 (m, 2H), 1.46 (s, 9H), 1.25 (d, J=6.0 Hz, 3H). LCMS m/z=257.1 [M+H-^{t}Bu]⁺.

### 2. Synthesis of N-((1R,3r,5S)-8-azabicyclo[3.2.1]octan-3-yl)-3-hydroxybutanamide

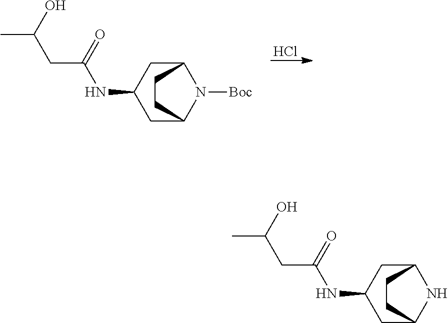 [SMILES: CC(O)CC(=O)N[C@H]1C[C@H]2CC[C@@H](C1)N2C(=O)OC(C)(C)C ; Cl ; CC(O)CC(=O)N[C@H]1C[C@H]2CC[C@@H](C1)N2]

A solution of tert-butyl (1R,3r,5S)-3-((3-hydroxybutyl)amino)-8-azabicyclo[3.2.1]octane-8-carboxylate (380.00 mg, 1.22 mmol) in HCl/MeOH (4 M, 6 mL) was stirred at 20° C. for 1 h. Evaporation in vacuo gave crude N-((1R,3r,5S)-8-azabicyclo[3.2.1]octan-3-yl)-3-hydroxybutanamide (300 mg, Hydrochloride) as a colorless oil. ¹H NMR (400 MHZ, CD₃OD) δ ppm 4.21-4.09 (m, 1H), 4.05-3.96 (m, 3H), 2.41-2.22 (m, 6H), 2.17-2.03 (m, 4H), 1.22 (d, J=6.0 Hz, 3H). LCMS m/z=212.9 [M+H]⁺.

### 3. Synthesis of N-((1R,3r,5S)-8-((4-(difluoromethoxy)phenyl)sulfonyl)-8-azabicyclo[3.2.1]octan-3-yl)-3-hydroxybutanamide

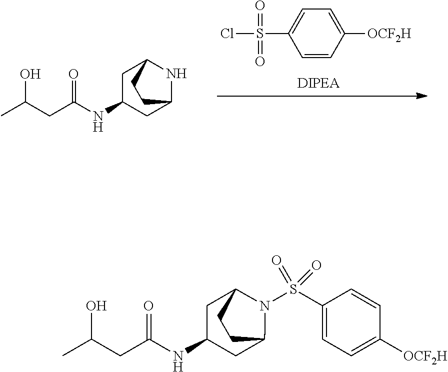

To a solution of 4-(((1R,3r,5S)-8-azabicyclo[3.2.1]octan-3-yl)amino)butan-2-ol (300.00 mg, 1.21 mmol, Hydrochloride) and DIPEA (623.47 mg, 4.82 mmol, 840.26 μL) in DCM (6 mL) was added 4-(difluoromethoxy) benzenesulfonyl chloride (438.93 mg, 1.81 mmol, 288.20 μL) at 0˜5° C. The mixture was stirred at 20° C. for 2 h. The mixture was diluted with water (15 mL) and extracted with DCM (20 mL×3). The combined organic phase was washed with brine (40 mL×3), dried over anhydrous sodium sulfate, filtered and concentrated in vacuo. The crude material was purified by flash column (MeOH in DCM=0.5%˜2%) to give the desired compound (320 mg, 63% yield) as a white solid. ¹H NMR (400 MHZ, CDCl₃) δ ppm 7.91-7.85 (m, 2H), 7.25-7.17 (m, 2H), 6.80-6.40 (m, 2H), 4.24 (br s, 2H), 4.19-4.08 (m, 2H), 2.94 (br s, 1H), 2.37-2.18 (m, 4H), 1.85-1.72 (m, 6H), 1.22 (d, J=6.0 Hz, 3H). LCMS m/z=419.0 [M+H]⁺.

### 4. Synthesis of rac-4-(((1R,3r,5S)-8-((4-(difluoromethoxy)phenyl)sulfonyl)-8-azabicyclo[3.2.1]octan-3-yl)amino)butan-2-ol

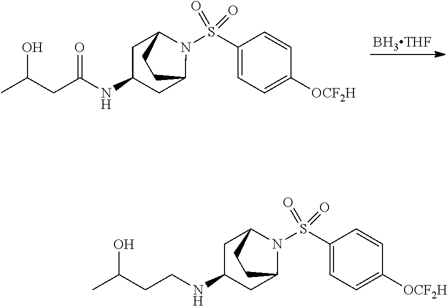

To a solution of N-((1R,3r,5S)-8-((4-(difluoromethoxy)phenyl)sulfonyl)-8-azabicyclo[3.2.1]octan-3-yl)-3-hydroxybutanamide (100 mg, 238.97 μmol) in THF (8 mL) was added BH₃·DMS (10 M, 95.59 μL) at 20° C. and the reaction mixture was stirred at 70° C. for 1 h under nitrogen. The mixture was quenched with methanol (10 mL) at 20° C., and then heated at 70° C. for 0.5 h. Concentration in vacuo gave the crude product which was purified by pre-HPLC (Column: Boston Prime C18 150*30 mm*5 μm, Condition: water (0.05% ammonia hydroxide v/v)-ACN, 50% ˜80%, Flow Rate (ml/min): 30)) to afford the title compound (111 mg, 37% yield) as a white solid. ¹H NMR (400 MHZ, CD₃OD) δ ppm 7.94-7.89 (m, 2H), 7.32-7.26 (m, 2H), 7.00 (t, J=73.2 Hz, 1H), 4.17 (br s, 2H), 3.90-3.80 (m, 1H), 2.89-2.81 (m, 1H), 2.66 (t, J=6.4 Hz, 2H), 2.18-2.03 (m, 2H), 1.97-1.86 (m, 2H), 1.79-1.69 (m, 2H), 1.64-1.48 (m, 2H), 1.47-1.41 (m, 2H), 1.14 (d, J=6.0 Hz, 3H). LCMS m/z=405.1 [M+H]⁺.

### Example 60: rac-4-(((1R,3s,5S)-8-((4-(difluoromethoxy)phenyl)sulfonyl)-8-azabicyclo[3.2.1]octan-3-yl)amino)butan-2-ol

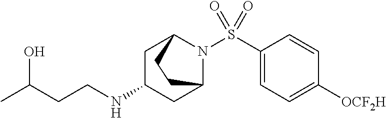

rac-4-(((1R,3s,5S)-8-((4-(Difluoromethoxy)phenyl)sulfonyl)-8-azabicyclo[3.2.1]octan-3-yl)amino)butan-2-ol was prepared as a white solid (200 mg, 42%) following an analogous method to that described for Example 59, using tert-butyl (1R,3s,5S)-3-(3-hydroxybutanamido)-8-azabicyclo[3.2.1]octane-8-carboxylate and 3-Hydroxybutanoic acid in step 1. 1H NMR (400 MHZ, METHANOL-d4) δ=7.99-7.95 (m, 2H), 7.34 (d, J=8.8 Hz, 2H), 7.24-6.86 (m, 1H), 4.41 (br s, 2H), 3.93-3.84 (m, 1H), 3.60-3.47 (m, 1H), 3.16-3.09 (m, 2H), 2.19-2.14 (m, 2H), 1.86-1.78 (m, 3H), 1.72-1.64 (m, 3H), 1.58-1.52 (m, 2H), 1.23 (d, J=6.4 Hz, 3H). LCMS m/z=405.2 [M+H]⁺.

### Examples 61 and 62: (1R,3r)-3-((((1R,3r,5S)-8-((4-(difluoromethoxy)phenyl)sulfonyl)-8-azabicyclo[3.2.1]octan-3 yl)amino)methyl)cyclobutan-1-ol and (1R,3r)-3-((((1R,3s,5S)-8-((4-(difluoromethoxy)phenyl)sulfonyl)-8-azabicyclo[3.2.1]octan-3-yl)amino)methyl)cyclobutan-1-ol

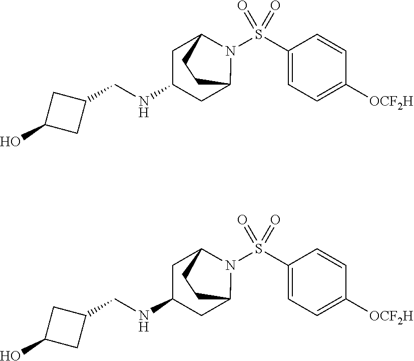

### *Stereochemistry Arbitrarily Assigned

(1R,3r)-3-((((1R,3r,5S)-8-((4-(Difluoromethoxy)phenyl)sulfonyl)-8-azabicyclo[3.2.1]octan-3 yl)amino)methyl)cyclobutan-1-ol and (1R,3r)-3-((((1R,3s,5S)-8-((4-(difluoromethoxy)phenyl)sulfonyl)-8-azabicyclo[3.2.1]octan-3-yl)amino)methyl)cyclobutan-1-ol were prepared following General Procedure C using (1r,3r)-3-(aminomethyl)cyclobutan-1-ol (HCl salt) as the secondary amine. The crude reaction mix was purified by SFC separation (Column: DAICEL CHIRALPAK AS (250 mm*50 mm, 10 μm); Conditions: 0.1% NH₃H₂O ETOH; Begin B: 40%; End B: 40%; FlowRate (ml/min): 200) to give (1R,3r)-3-((((1R,3r,5S)-8-((4-(difluoromethoxy)phenyl)sulfonyl)-8-azabicyclo[3.2.1]octan-3-yl)amino)methyl)cyclobutan-1-ol (233.15 mg., 23% yield) and crude (1R,3r)-3-((((1R,3s,5S)-8-((4-(difluoromethoxy)phenyl)sulfonyl)-8-azabicyclo[3.2.1]octan-3 yl)amino)methyl)cyclobutan-1-ol was purified further by prep-HPLC (Column: Boston Green ODS 150*30 mm*5 μm; Condition: water (HCl)-CAN; Begin B: 20; End B: 40; FlowRate (ml/min): 25) to affort the second isomer (200 mg 20% yield) as a white solid.
*Peak 1, Example 61: ¹H NMR (400 MHZ, CD₃OD) δ ppm 7.91 (d, J=8.8 Hz, 2H), 7.31-7.27 (m, 2H), 7.01 (t, J=72.8 Hz, 1H), 4.32-4.24 (m, 1H), 4.17 (br s, 2H), 2.91-2.87 (m, 1H), 2.57 (d, J=7.6 Hz, 2H), 2.11-2.07 (m, 1H), 2.06-1.97 (m, 8H), 1.73-1.68 (m, 2H), 1.44-1.41 (m, 2H). LCMS m/z=417.1 [M+H]⁺.
*Peak 2, Example 62: 1H NMR (400 MHZ, CD₃OD) δ ppm 7.95 (d, J=8.8 Hz, 2H), 7.32 (d, J=8.8 Hz, 2H), 7.03 (t. J=72.8 Hz, 1H), 4.43-4.30 (m, 3H), 3.51 (m, 1H), 3.08 (d, J=8.0 Hz, 2H), 2.59-2.45 (m, 1H), 2.22-2.10 (m, 6H), 1.89-1.75 (m, 2H), 1.69-1.51 (m, 4H). LCMS m/z=417.1 [M+H]⁺.

### Examples 63 and 64: (1S,3s)-3-((((1R,3s,5S)-8-((4-(difluoromethoxy)phenyl)sulfonyl)-8-azabicyclo[3.2.1]octan-3-yl)amino)methyl)cyclobutan-1-ol and (1S,3s)-3-((((1R,3r,5S)-8-((4-(difluoromethoxy)phenyl)sulfonyl)-8-azabicyclo[3.2.1]octan-3-yl)amino)methyl)cyclobutan-1-ol

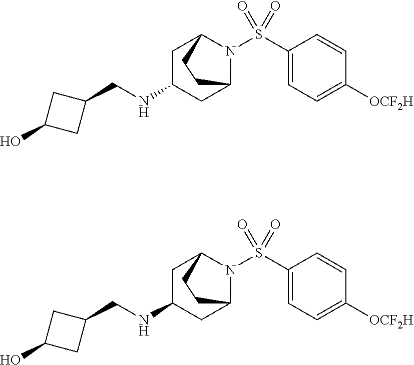

### *Stereochemistry Arbitrarily Assigned

(1S,3s)-3-((((1R,3s,5S)-8-((4-(difluoromethoxy)phenyl)sulfonyl)-8-azabicyclo[3.2.1]octan-3-yl)amino)methyl)cyclobutan-1-ol and (1S,3s)-3-((((1R,3r,5S)-8-((4-(difluoromethoxy)phenyl)sulfonyl)-8-azabicyclo[3.2.1]octan-3-yl)amino)methyl)cyclobutan-1-ol were prepared following General Procedure C using (1s,3r)-3-(aminomethyl)cyclobutan-1-ol (HCl salt) as the secondary amine. The crude reaction mix was purified by flash silica gel chromatography (DCM/MeOH=10/1) to give the mixture of isomers as a white solid. Isomers were then separated by SFC (Column DAICEL CHIRALPAK IG (250 mm*30 mm, 10 μm); Condition: 0.1% NH₃·H₂O MEOH; Begin B: 45%; End B: 45%; Flow Rate (ml/min): 80) to give (1S,3s)-3-((((1R,3s,5S)-8-((4-(difluoromethoxy)phenyl)sulfonyl)-8-azabicyclo[3.2.1]octan-3-yl)amino)methyl)cyclobutan-1-ol (75.75 mg 6% yield) as a white solid and (1S,3s)-3-((((1R,3r,5S)-8-((4-(difluoromethoxy)phenyl)sulfonyl)-8-azabicyclo[3.2.1]octan-3-yl)amino)methyl)cyclobutan-1-ol (237.56 mg, 19% yield) as a white solid.
*Peak 1, Example 63: ¹H NMR (500 MHZ, DMSO-d₆) δ ppm 7.95-7.84 (m, 2H), 7.55-7.23 (m, 3H), 4.88-4.88 (m, 1H), 4.16 (br s, 2H), 4.18-4.13 (m, 1H), 3.91-3.74 (m, 1H), 2.46-2.42 (m, 2H), 2.24-2.16 (m, 2H), 1.88-1.80 (m, 2H), 1.75-1.60 (m, 1H), 1.52-1.44 (m, 2H), 1.40-1.37 (m, 2H), 1.32-1.15 (m, 5H). LCMS m/z=417.1 [M+H]⁺.
*Peak 2, Example 64: ¹H NMR 500 MHZ, DMSO-d₆) δ ppm 7.92-7.87 (m, 2H), 7.56-7.19 (m, 3H), 4.85 (d, J=6.5 Hz, 1H), 4.08 (br s, 2H), 3.90-3.83 (m, 1H), 2.79 (t. J=5.5 Hz, 1H), 2.42-2.40 (m, 2H), 2.24-2.16 (m, 2H), 1.98-1.92 (m, 2H), 1.86-1.80 (m, 2H), 1.73-1.66 (m, 3H), 1.43-1.34 (m, 2H), 1.28 (br s, 1H), 1.23-1.15 (m, 2H). LCMS m/z=417.1 [M+H]⁺.

### Example 65: (1R,3r,5S)-8-((4-(difluoromethoxy)phenyl)sulfonyl)-N-methyl-N-(tetrahydro-2H-pyran-4-yl)-8-azabicyclo[3.2.1]octan-3-amine

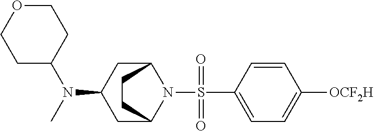

### 1. Synthesis of (1R,3r,5S)-8-((4-(difluoromethoxy)phenyl)sulfonyl)-N-(tetrahydro-2H-pyran-4-yl)-8-azabicyclo[3.2.1]octan-3-amine

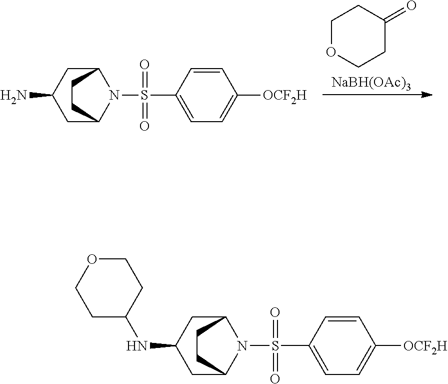

(1R,3r,5S)-8-((4-(Difluoromethoxy)phenyl)sulfonyl)-8-azabicyclo[3.2.1]octan-3-amine (Intermediate 3, 100 mg, 0.3 mmol) as hydrochloride salt and tetrahydro-4H-pyran-4-one (36 μL, 0.4 mmol) were taken up in DCM (2 mL). To this was added DIPEA (160 μL, 0.9 mmol), with stirring. After 10 min, acetic acid (52 μL, 0.9 mmol), was added with stirring. After 10 min, sodium triacetoxyborohydride (255 mg, 1.2 mmol) was added with stirring. Additional DCM (2 mL) was added, and the reaction was stirred overnight at room temperature. Reaction mixture was poured into saturated sodium bicarbonate, extracted with ethyl acetate, and the organics were evaporated. Residue was purified by silica gel chromatography using 0-100% 3:1 ethyl acetate/ethanol (+2% NH₄OH) in heptanes as eluent to afford (1R,3r,5S)-8-((4-(difluoromethoxy)phenyl)sulfonyl)-N-(tetrahydro-2H-pyran-4-yl)-8-azabicyclo[3.2.1]octan-3-amine (117 mg, 93%). ¹H NMR (400 MHZ, CD₃OD) δ (ppm): 7.85-7.99 (m, 2H), 7.26-7.33 (m, 2H), 6.79-7.21 (m, 1H), 4.21 (dt, J=4.83, 2.48 Hz, 2H), 3.93 (dt, J=10.10, 2.10 Hz, 2H), 3.40 (td, J=11.86, 2.13 Hz, 2H), 3.18 (tt, J=6.65, 3.51 Hz, 1H), 2.81 (tt, J=10.95, 4.11 Hz, 1H), 2.21 (dt, J=13.68, 5.96 Hz, 2H), 1.91-1.97 (m, 3H), 1.78-1.85 (m, 2H), 1.57-1.65 (m, 2H), 1.41-1.52 (m, 2H) 1.25-1.38 (m, 1H). LCMS m/z=417.2 [M+H]⁺.

### 2. Synthesis of (1R,3r,5S)-8-((4-(difluoromethoxy)phenyl)sulfonyl)-N-methyl-N-(tetrahydro-2H-pyran-4-yl)-8-azabicyclo[3.2.1]octan-3-amine

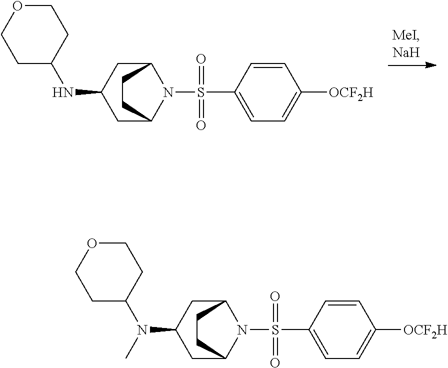

(1R,3r,5S)-8-((4-(Difluoromethoxy)phenyl)sulfonyl)-N-(tetrahydro-2H-pyran-4-yl)-8-azabicyclo[3.2.1]octan-3-amine (40 mg, 0.1 mmol) was taken up in DMF (1 mL). To this was added sodium hydride (60% purity, 8 mg, 0.2 mmol) followed by iodomethane (24 μL, 0.4 mmol). The reaction was stirred at room temperature for 45 min to 1.5 h. The reaction was quenched by addition of saturated sodium bicarbonate solution, extracted with ethyl acetate, and the organics were evaporated. The residue was dissolved in DMSO purified by prep-HPLC (Waters SunFire Prep C18 5 μm OBD 30×100 mm; 5-55% MeCN in water (+TFA)) to afford (1R,3r,5S)-8-((4-(difluoromethoxy)phenyl)sulfonyl)-N-methyl-N-(tetrahydro-2H-pyran-4-yl)-8-azabicyclo[3.2.1]octan-3-amine (trifluoroacetate salt) as a white solid (19 mg, 36%). LCMS m/z=431.3 [M+H]⁺, t_{R}=1.38 min.

### Example 66: (1R,3s,5S)-8-((4-(difluoromethoxy)phenyl)sulfonyl)-N-methyl-N-(tetrahydro-2H-pyran-4-yl)-8-azabicyclo[3.2.1]octan-3-amine

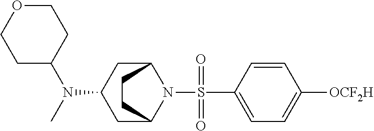

The title compound (trifluoroacetate salt) was prepared as a clear oil (18 mg, 38% over two steps) from (1R,3s,5S)-8-((4-(difluoromethoxy)phenyl)sulfonyl)-8-azabicyclo[3.2.1]octan-3-amine (Intermediate 4 as hydrochloride salt) using an analogous procedure to that described for Example 65. LCMS m/z=431.3 [M+H]⁺, t_{R}=1.39 min.

### Example 67: 4-(((1R,3s,5S)-8-((4-(Difluoromethoxy)phenyl)sulfonyl)-8-azabicyclo[3.2.1]octan-3-yl)methyl)morpholine

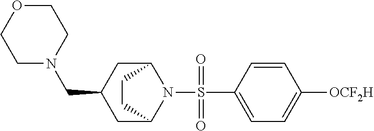

4-(((1R,3s,5S)-8-((4-(Difluoromethoxy)phenyl)sulfonyl)-8-azabicyclo[3.2.1]octan-3-yl)methyl)morpholine was prepared as a white solid (48 mg, 89%) from 4-(((1R,3s,5S)-8-azabicyclo[3.2.1]octan-3-yl)methyl)morpholine dihydrochloride (Intermediate 7) and 4-(difluoromethoxy) benzenesulfonyl chloride using an analogous method to that described for Example 35, and purified by prep-HPLC (Welch Xtimate C18 25×150 mm, 5 μm; 33-63% MeCN/water (10 mM NH₄HCO₃) to afford the desired compound (48 mg, 89%). LCMS m/z=417.2 [M+H]⁺. t_{R}=0.59 min.

### Example 68 and 69: (1R,3s,5S)-8-((2-Methyl-6-(trifluoromethyl)pyridin-3-yl)sulfonyl)-N-(2-oxaspiro[3.3]heptan-6-yl)-8-azabicyclo[3.2.1]octan-3-amine and (1R,3r,5S)-8-((2-methyl-6-(trifluoromethyl)pyridin-3-yl)sulfonyl)-N-(2-oxaspiro[3.3]heptan-6-yl)-8-azabicyclo[3.2.1]octan-3-amine

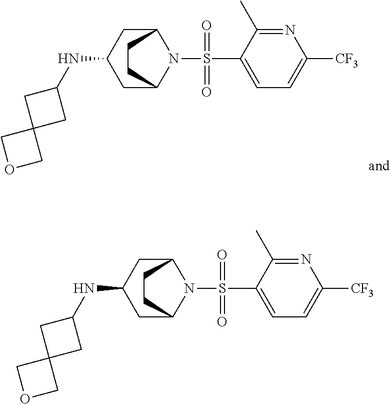 [SMILES: Cc1nc(C(F)(F)F)ccc1S(=O)(=O)N1[C@@H]2CC[C@H]1C[C@H](NC1CC3(COC3)C1)C2 ; Cc1nc(C(F)(F)F)ccc1S(=O)(=O)N1[C@@H]2CC[C@H]1C[C@@H](NC1CC3(COC3)C1)C2]

### *Stereochemistry Arbitrarily Assigned

(1R,5S)-8-((2-Methyl-6-(trifluoromethyl)pyridin-3-yl)sulfonyl)-N-(2-oxaspiro[3.3]heptan-6-yl)-8-azabicyclo[3.2.1]octan-3-amine was prepared from 8-((2-methyl-6-(trifluoromethyl)pyridin-3-yl)sulfonyl)-8-azabicyclo[3.2.1]octan-3-one (Intermediate 6) and 2-oxaspiro[3.3]heptan-6-amine using an analogous method to that described for Example 19, purification by column chromatography (24 g SiO₂, 20-50-100% EtOAc/EtOH (3/1) in heptane) afforded:
*Peak 1, Example 68; white solid (35 mg, 36%). ¹H NMR (400 MHZ, CD₃OD) δ (ppm): 8.53 (d, J=9.0 Hz, 1H), 7.84 (d, J=8.0 Hz, 1H), 4.72 (s, 2H), 4.59 (s, 2H), 4.16 (br s, 2H), 3.05-3.17 (m, 1H), 2.84-2.94 (m, 4H), 2.46-2.57 (m, 2H), 2.12-2.22 (m, 2H), 1.99-2.11 (m, 2H), 1.85-1.97 (m, 4H), 1.65 (br d, J=15.0 Hz, 2H). LCMS m/z=446.2 [M+H]⁺.
*Peak 2, Example 69; white solid (22 mg, 23%). ¹H NMR (400 MHZ, CD₃OD) δ (ppm): 8.54 (d, J=8.0 Hz, 1H), 7.85 (d, J=8.0 Hz, 1H), 4.21 (br s, 2H), 3.63-3.75 (m, 3H), 2.92 (s, 3H), 2.71-2.78 (m, 1H), 2.59 (br s, 2H), 2.17-2.23 (m, 2H), 2.04-2.14 (m, 2H), 1.93 (dt, J=6.3, 3.4 Hz, 2H), 1.86 (br s, 2H), 1.58 (br d, J=15.0 Hz, 2H), 1.45 (dd, J=5.0, 2.0 Hz, 2H). LCMS m/z=446.2 [M+H]⁺.

### Example 70: 8-((4-(Difluoromethoxy)phenyl)sulfonyl)-N—((R)-tetrahydrofuran-3-yl)-8-azabicyclo[3.2.1]octan-3-amine

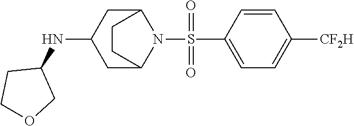 [SMILES: O=S(=O)(c1ccc(C(F)F)cc1)N1C2CCC1CC(N[C@@H]1CCOC1)C2]

8-((4-(Difluoromethoxy)phenyl)sulfonyl)-N—((R)-tetrahydrofuran-3-yl)-8-azabicyclo[3.2.1]octan-3-amine was prepared from 8-((4-(difluoromethoxy)phenyl)sulfonyl)-8-azabicyclo[3.2.1]octan-3-one (Intermediate 5) and (R)-tetrahydrofuran-3-amine using an analogous method to that described for Example 19, purification by column chromatography (12 g SiO₂, 50-100% EtOAc in heptane) afforded a white powder after lyophilization (15 mg, 19%). ¹H NMR (400 MHZ, CD₃OD) δ (ppm): 7.86-8.00 (m, 2H), 7.31 (d, J=8.8 Hz, 2H), 6.76-7.24 (m, 1H), 4.19 (br s, 2H), 3.68-3.97 (m, 3H), 3.37-3.52 (m, 2H), 2.99 (br t, J=6.0 Hz, 1H), 1.96-2.21 (m, 5H), 1.60-1.78 (m, 3H), 1.34-1.53 (m, 2H). LCMS m/z=403.1 [M+H]⁺.

### Example 71: (1R,3r,5S)-8-((4-(Difluoromethoxy)phenyl)sulfonyl)-N-((3-fluorooxetan-3-yl)methyl)-8-azabicyclo[3.2.1]octan-3-amine

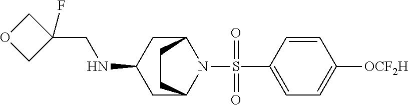

(1R,3r,5S)-8-((4-(Difluoromethoxy)phenyl)sulfonyl)-N-((3-fluorooxetan-3-yl)methyl)-8-azabicyclo[3.2.1]octan-3-amine (stereochemistry arbitrarily assigned) was prepared from 8-((4-(difluoromethoxy)phenyl)sulfonyl)-8-azabicyclo[3.2.1]octan-3-one (Intermediate 5) and (3-fluorooxetan-3-yl) methanamine using an analogous method to that described for Example 19, purification by column chromatography (12 g SiO₂, 20-50-100% EtOAc/EtOH 3/1 in heptane) afforded a white powder after lyophilization (6.3 mg, 7%, more polar isomer, stereochemistry arbitrarily assigned). LCMS m/z=421.1 [M+H]⁺. t_{R}=0.58 min.

### Example 72: (1R,3r,5S)-8-((4-(Difluoromethoxy)phenyl)sulfonyl)-N-(oxetan-3-ylmethyl)-8-azabicyclo[3.2.1]octan-3-amine

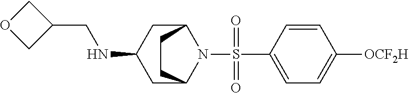

(1R,3r,5S)-8-((4-(Difluoromethoxy)phenyl)sulfonyl)-N-(oxetan-3-ylmethyl)-8-azabicyclo[3.2.1]octan-3-amine was prepared from (1R,3r,5S)-8-((4-(difluoromethoxy)phenyl)sulfonyl)-8-azabicyclo[3.2.1]octan-3-amine (Intermediate 3) and oxetane-3-carbaldehyde using an analogous method to that described for Example 19, and purified by prep-HPLC (Welch Xtimate C18 25×150 mm, 5 μm; 33-63% MeCN/water (10 mM NH₄HCO₃) to afford the desired compound (48 mg, 76%). LCMS m/z=403.1 [M+H]⁺. t_{R}=0.56 min.

### Example 73: 8-((4-(Difluoromethoxy)phenyl)sulfonyl)-N-(tetrahydro-2H-pyran-4-yl)-8-azabicyclo[3.2.1]octan-3-amine

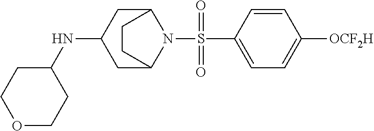

8-((4-(Difluoromethoxy)phenyl)sulfonyl)-N-(tetrahydro-2H-pyran-4-yl)-8-azabicyclo[3.2.1]octan-3-amine was prepared from 8-((4-(difluoromethoxy)phenyl)sulfonyl)-8-azabicyclo[3.2.1]octan-3-one (Intermediate 5) and tetrahydro-2H-pyran-4-amine using an analogous method to that described for Example 19, purified by prep-HPLC (Welch Xtimate C18 25×150 mm, 5 μm; 33-63% MeCN/water (10 mM NH₄HCO₃) to afford the desired compound (18 mg, 22%). LCMS m/z=417.2 [M+H]⁺. t_{R}=0.58 min.

### Example 74: 8-((4-(Difluoromethoxy)phenyl)sulfonyl)-N-(oxetan-3-yl)-8-azabicyclo[3.2.1]octan-3-amine

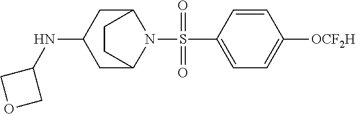

8-((4-(Difluoromethoxy)phenyl)sulfonyl)-N-(oxetan-3-yl)-8-azabicyclo[3.2.1]octan-3-amine was prepared from 8-((4-(difluoromethoxy)phenyl)sulfonyl)-8-azabicyclo[3.2.1]octan-3-one (Intermediate 5) and cyclobutanamine using an analogous method to that described for Example 19, purified by prep-HPLC (Welch Xtimate C18 25×150 mm, 5 μm; 33-63% MeCN/water (10 mM NH₄HCO₃) to afford the desired compound (61 mg, 67%). LCMS m/z=389.1 [M+H]⁺. t_{R}=0.55 min.

### Example 75: (1R,3s,5S)-8-((2-Methyl-6-(trifluoromethyl)pyridin-3-yl)sulfonyl)-N-(tetrahydro-2H-pyran-4-yl)-8-azabicyclo[3.2.1]octan-3-amine

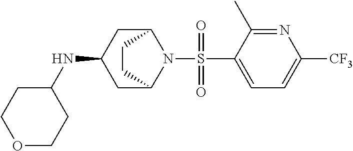 [SMILES: Cc1nc(C(F)(F)F)ccc1S(=O)(=O)N1[C@@H]2CC[C@H]1C[C@H](NC1CCOCC1)C2]

(1R,3s,5S)-8-((2-Methyl-6-(trifluoromethyl)pyridin-3-yl)sulfonyl)-N-(tetrahydro-2H-pyran-4-yl)-8-azabicyclo[3.2.1]octan-3-amine was prepared from (1R,3s,5S)-8-((2-methyl-6-(trifluoromethyl)pyridin-3-yl)sulfonyl)-8-azabicyclo[3.2.1]octan-3-amine (Intermediate 9) and tetrahydro-4H-pyran-4-one using an analogous method to that described for Example 19, purification by column chromatography (12 g SiO₂, 50-100% EtOAc/EtOH 3/1 in heptane) afforded a white sticky solid (65 mg, 63%). ¹H NMR (METHANOL-d4, 400 MHZ): δ (ppm) 8.55 (d, J=8.5 Hz, 1H), 7.84 (d, J=8.0 Hz, 1H), 4.28 (dd, J=4.0, 3.0 Hz, 2H), 3.94 (dd, J=11.0, 4.0 Hz, 2H), 3.40 (td, J=12.0, 2.0 Hz, 2H), 3.16 (tt, J=11.3, 5.5 Hz, 1H), 2.95 (s, 3H), 2.71-2.88 (m, 1H), 1.94-2.05 (m, 4H), 1.78-1.87 (m, 4H), 1.45-1.55 (m, 2H), 1.30-1.43 (m, 2H). LCMS m/z=434.2 [M+H]⁺

### Example 76: 1-(((1R,3s,5S)-8-((4-(Difluoromethoxy)phenyl)sulfonyl)-8-azabicyclo[3.2.1]octan-3-yl)amino)-2-methylpropan-2-ol

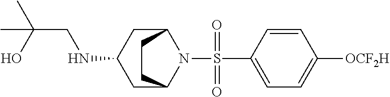

1-(((1R,3s,5S)-8-((4-(Difluoromethoxy)phenyl)sulfonyl)-8-azabicyclo[3.2.1]octan-3-yl)amino)-2-methylpropan-2-ol was prepared from (1R,3s,5S)-8-((4-(difluoromethoxy)phenyl)sulfonyl)-8-azabicyclo[3.2.1]octan-3-amine (Intermediate 4) and 2,2-dimethyloxirane using an analogous method to that described for Example 17, purification by column chromatography (12 g SiO₂, 100% EtOAc to 100% EtOAc/EtOH 3/1) afforded a white powder after lyophilization (30 mg, 74%). ¹H NMR (400 MHZ, CD₃OD) δ (ppm): 7.88-8.05 (m, 2H), 7.33 (d, J=7.3 Hz, 2H), 6.77-7.24 (m, 1H), 4.34 (br s, 2H), 3.07-3.24 (m, 1H), 2.72 (s, 2H), 2.08 (br d, J=13.1 Hz, 2H), 1.45-1.81 (m, 6H), 1.24 (d, J=1.0 Hz, 6H) LCMS m/z=405.1 [M+H]⁺.

### Example 77: 1-(((1R,3r,5S)-8-((4-(Difluoromethoxy)phenyl)sulfonyl)-8-azabicyclo[3.2.1]octan-3-yl)amino)-2-methylpropan-2-ol

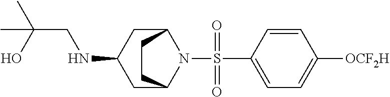

1-(((1R,3r,5S)-8-((4-(Difluoromethoxy)phenyl)sulfonyl)-8-azabicyclo[3.2.1]octan-3-yl)amino)-2-methylpropan-2-ol was prepared from (1R,3r,5S)-8-((4-(Difluoromethoxy)phenyl)sulfonyl)-8-azabicyclo[3.2.1]octan-3-amine (Intermediate 3) and 2,2-dimethyloxirane using an analogous method to that described for Example 17, purification by column chromatography (12 g SiO₂, 100% EtOAc) afforded a white powder after lyophilization (12 mg, 30%). ¹H NMR (400 MHZ, CD₃OD) δ (ppm): 7.94 (dd, J=8.8, 1.0 Hz, 2H), 7.31 (d, J=7.8 Hz, 2H), 6.77-7.22 (m, 1H), 4.18 (br s, 2H), 2.89 (br t, J=5.8 Hz, 1H), 2.48 (s, 2H), 1.97-2.17 (m, 4H), 1.76 (br d, J=14.1 Hz, 2H), 1.34-1.55 (m, 2H), 1.20 (d, J=1.0 Hz, 6H). LCMS m/z=405.1 [M+H]⁺.

### ASSAYS

### EBP Functional Assay

The EBP immunoaffinity (IA) LC-MS assay measures the potency of small molecule inhibitors of EBP by quantifying their concentration-dependent changes in the enzyme's substrate and product using liquid chromatography atmospheric pressure chemical ionization multiple reaction monitoring mass spectrometry (LC-APCI MRM MS). HEK293T cells were utilized as the source of EBP enzyme. The enzyme was incubated with the small molecule inhibitors at variable concentrations for 30 min. Deuterated form of EBP substrate, zymosterol-d5 (Avanti Polar Lipids, Cat #700068P-1 mg), was then added and the plate was incubated at 37° C. for 4 h. Finally, the sterol isomers were extracted and injected to LC-APCI MRM MS. MRM transition used for the quantification for both zymosterol and dihydrolathosterol (substrate and product of EBP enzymatic reaction, respectively) is 372.3-203.2, CE 30 and DP 80 in positive ion mode. Percent conversion of the zymosterol-d5 to dehydrolathosterol-d5 was used to derive IC₅₀ curves. Tasin-1 (1′-[(4-Methoxyphenyl)sulfonyl]-4-methyl-1,4′-bipiperidine, CAS 792927-06-1) was used as the reference small molecule inhibitor.

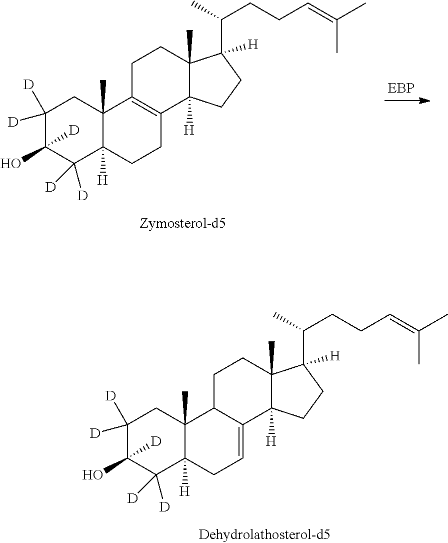

Percent conversion versus the compound concentration data were fit to the following 4-parameter logistic model to generate IC₅₀ curves:

$$y = LA + \frac{UA - LA}{1 + 1 {0}^{( log {IC}_{50} - x ) \cdot Slope}}$$

### DATA FOR EXAMPLES

| Example | EBP Functional |
|---|---|
| Number | Assay |
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | ++ |
| 35 | +++ |
| 36 | +++ |
| 37 | +++ |
| 38 | +++ |
| 39 | ++ |
| 40 | + |
| 41 | +++ |
| 42 | + |
| 43 | ++ |
| 44 | + |
| 45 | + |
| 46 | + |
| 47 | ++ |
| 48 | + |
| 49 | ++ |
| 50 | ++ |
| 51 | + |
| 52 | +++ |
| 53 | ++ |
| 54 | + |
| 55 | +++ |
| 56 | + |
| 57 | ++ |
| 58 | +++ |
| 59 | ++ |
| 60 | + |
| 61 | ++ |
| 62 | ++ |
| 63 | + |
| 64 | ++ |
| 65 | +++ |
| 66 | + |
| 67 | +++ |
| 68 | +++ |
| 69 | ++ |
| 70 | ++ |
| 71 | + |
| 72 | +++ |
| 73 | +++ |
| 74 | N/A |
| 75 | + |
| 76 | + |
| 77 | ++ |

*N/A means >2000 nM; + means equal to or less than 2000 nm and greater than 150 nM; ++ means equal to or greater than 50 nm and less than or equal to150 nM; +++ means <50 nM.

What is claimed is:

1. A compound represented by Formula (I): 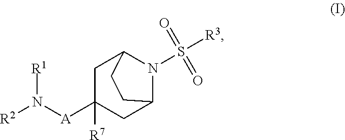
or a pharmaceutically acceptable salt thereof, wherein:
A is CH₂ or a bond;
R¹ and R² are each independently selected from H, C₁₋₆alkyl, C₃₋₈cycloalkyl, Het, —Z-Het, and —Z—C₃₋₈cycloalkyl, wherein the C₁₋₆alkyl, C₃₋₈cycloalkyl and Het are each optionally substituted with one or more R⁴, provided at least one of R¹ and R² is not H; or
R¹ and R² together with the nitrogen atom from which they are attached form a 4 to 10 membered heterocyclyl optionally substituted with one or more substituent R⁴;
Z is C₁₋₄alkyl optionally substituted with one or more halo
Het is 4 to 7 membered monocyclic heterocyclyl or 6 to 10 membered bicyclic heterocyclyl, each of which is optionally substituted with R⁴;
R³ is phenyl, 5 or 6-membered monocyclic heteroaryl, or 9 or 10-membered bicyclic heteroaryl, wherein the phenyl, 5 or 6-membered monocyclic heteroaryl, and 9 or 10-membered bicyclic heteroaryl are each optionally substituted with one or more substituent R⁵;
R⁴, for each occurrence, is independently C₁₋₆alkyl, halo, and —OR^{4a}, wherein the C₁₋₆alkyl is optionally substituted with —OR^{4a},
R^{4a} is H, C₁₋₆alkyl or C₁₋₆haloalkyl;
R⁵, for each occurrence, is independently, halo, —OR^{5a}, C₁₋₆alkyl, C₃₋₆cycloalkyl, 4 to 7-membered monocyclic heterocyclyl, 5- to 6-membered monocyclic heteroaryl, or phenyl, wherein the C₁₋₆alkyl, C₃₋₆cycloalkyl, and 5- to 6-membered monocyclic heteroaryl are each optionally substituted with one or more halo, C₁₋₄alkyl, or —OR^{5a};
or two R⁵ together with their intervening atoms form a 5 to 7-membered heterocycle optionally substituted with one or more R⁶ independently selected from C₁₋₆alkyl, oxo, halo, and —OR^{5a},
R^{5a} is H, C₁₋₆alkyl or C₁₋₆haloalkyl, wherein the C₁₋₆alkyl is optionally substituted with alkoxy;
R⁷ is H or OR^{7a};
R^{7a} is H, H, C₁₋₆alkyl or C₁₋₆haloalkyl, wherein the C₁₋₆alkyl is optionally substituted with alkoxy.

2. The compound of claim 1, or a pharmaceutically acceptable salt thereof, wherein:
R¹ and R² are each independently selected from H, C₁₋₆alkyl, C₃₋₈cycloalkyl, Het, —Z-Het, wherein the C₁₋₆alkyl, C₃₋₈cycloalkyl and Het are each optionally substituted with one or more R⁴, provided at least one of R¹ and R² is not H; or
R¹ and R² together with the nitrogen atom from which they are attached form a 4 to 10 membered heterocyclyl optionally substituted with one or more substituent R⁴;
Z is C₁₋₄alkyl optionally substituted with one or more halo
Het is 4 to 7 membered monocyclic heterocyclyl or 6 to 10 membered bicyclic heterocyclyl, each of which is optionally substituted with R⁴;
R³ is phenyl, 5 or 6-membered monocyclic heteroaryl, or 9 or 10-membered bicyclic heteroaryl, wherein the phenyl, 5 or 6-membered monocyclic heteroaryl, and 9 or 10-membered bicyclic heteroaryl are each optionally substituted with one or more substituent R⁵;
R⁴, for each occurrence, is independently C₁₋₆alkyl, halo, and —OR^{4a};
R^{4a} is H, C₁₋₆alkyl or C₁₋₆haloalkyl;
R⁵, for each occurrence, is independently, halo, —OR^{5a}, C₁₋₆alkyl, C₃₋₆cycloalkyl, 4 to 7-membered monocyclic heterocyclyl, or phenyl, wherein the C₁₋₆alkyl and C₃₋₆cycloalkyl are each optionally substituted with one or more halo, —OR^{5a};
or two R⁵ together with their intervening atoms form a 5 to 7-membered heterocycle optionally substituted with one or more R⁶ independently selected from C₁₋₆alkyl, oxo, halo, and —OR^{5a};
R^{5a} is H, C₁₋₆alkyl or C₁₋₆haloalkyl, wherein the C₁₋₆alkyl is optionally substituted with alkoxy;
R⁷ is H or OR^{7a};
R^{7a} is H, C₁₋₆alkyl or C₁₋₆haloalkyl, wherein the C₁₋₆alkyl is optionally substituted with alkoxy.

3. The compound of claim 1 or 2, wherein the compound is represented by Formula II: 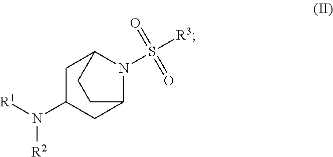
or a pharmaceutically acceptable salt thereof.

4. The compound of claim 3, wherein the compound is represented by Formulas (IIA) or (IIB): 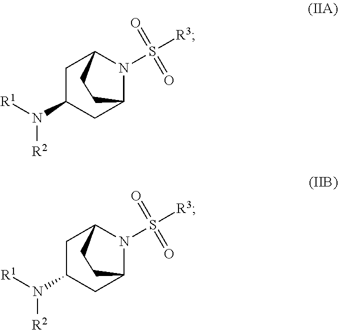
or a pharmaceutically acceptable salt thereof.

5. The compound of any one of claims 1 to 4, or a pharmaceutically acceptable salt thereof, wherein R³ is selected from the group consisting of phenyl, pyrazolyl, imidazolyl, oxazolyl, isoxazolyl, pyridinyl, pyrazolopyridinyl, pyrazolopyridinyl, benzotriazolyl, furanyl, oxadiazolyl, imidazopyridinyl, thiophenyl, thiazoyl, triazoyl, and indoyl, each of which is optionally substituted with one to three R⁵.

6. The compound of any one of claims 1 to 4, or a pharmaceutically acceptable salt thereof, wherein R³ is selected from the group consisting of phenyl, pyrazolyl, imidazolyl, oxazolyl, pyridinyl, pyrazolopyridinyl, pyrazolopyridinyl, benzotriazolyl, furanyl, oxadiazolyl, imidazopyridinyl, thiophenyl, and indoyl, each of which is optionally substituted with one to three R⁵.

7. The compound of any one of claims 1 to 4, or a pharmaceutically acceptable salt thereof, wherein R³ is represented by the following formula: 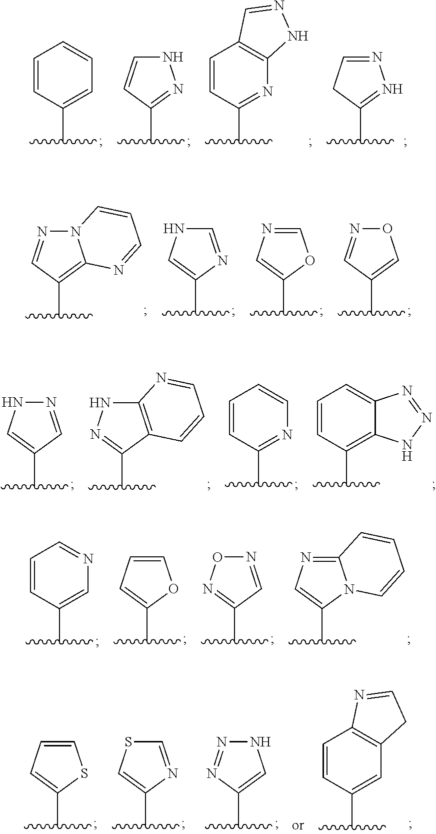
each of which is optionally substituted with one to three R⁵; or R³ is presented by the following formula: 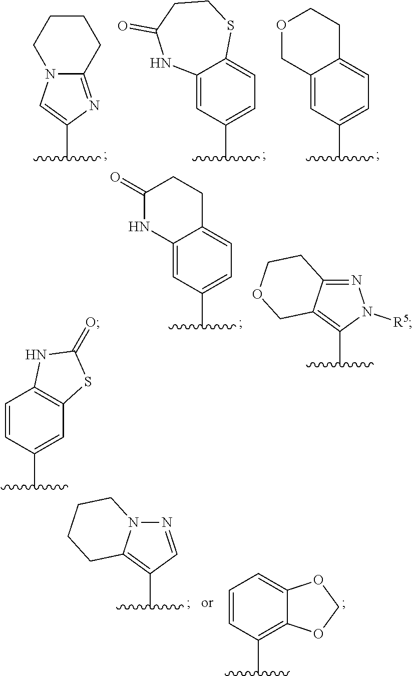
each of which is optionally substituted with one or two R⁶.

8. The compound of any one of claims 1 to 4, or a pharmaceutically acceptable salt thereof, wherein R³ is represented by the following formula: 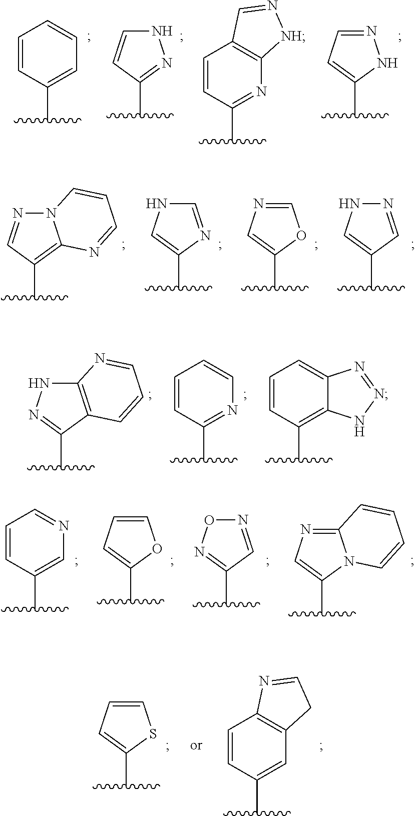
each of which is optionally substituted with one to three R⁵; or R³ is presented by the following formula: 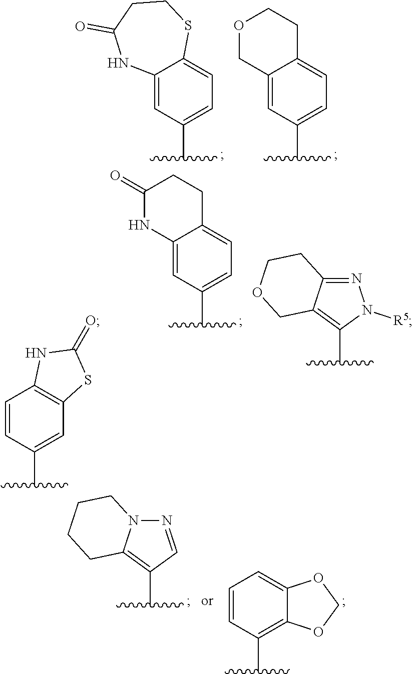
each of which is optionally substituted with one or two R⁶.

9. The compound of claim 5 or 6, or a pharmaceutically acceptable salt thereof, wherein R³ is selected from the group consisting of phenyl, pyrazolyl, imidazolyl, oxazolyl, and pyridinyl, each of which is optionally substituted with one to three R⁵.

10. The compound of claim 7 or 8, or a pharmaceutically acceptable salt thereof, wherein R³ is represented by the following formula: 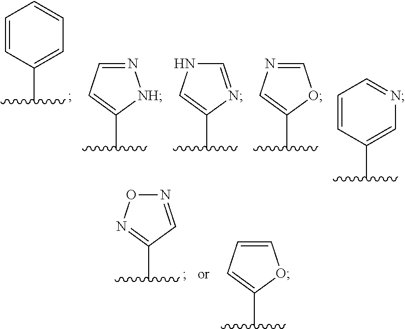
each of which is optionally substituted with one or two R⁵;
or R³ is presented by the following formula: 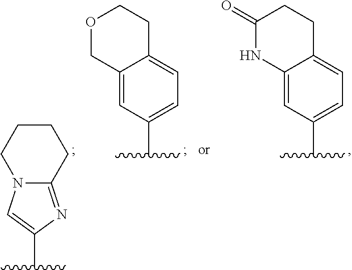 [SMILES: *c1cn2c(n1)CCCC2 ; *c1ccc2c(c1)COCC2 ; *c1ccc2c(c1)NC(=O)CC2]
each of which is optionally substituted with one or two R⁶.

11. The compound of claim 7, or a pharmaceutically acceptable salt thereof, wherein R³ is represented by the following formula:

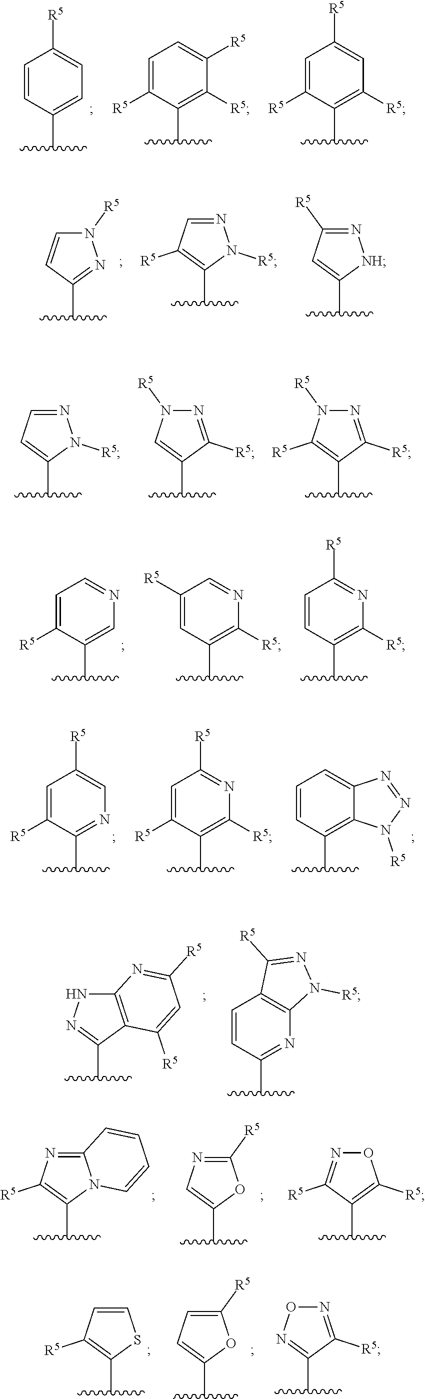

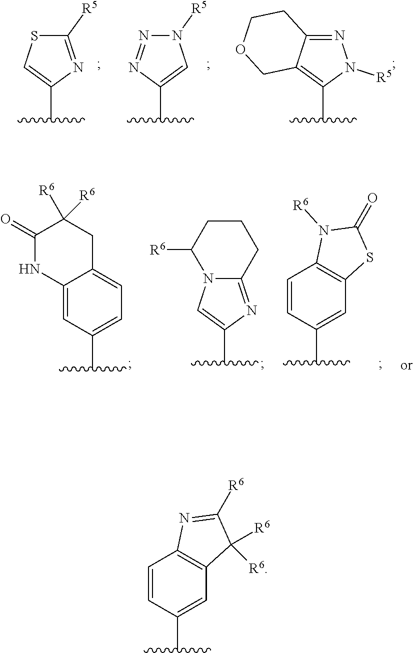

12. The compound of claim 8, or a pharmaceutically acceptable salt thereof, wherein R³ is represented by the following formula:

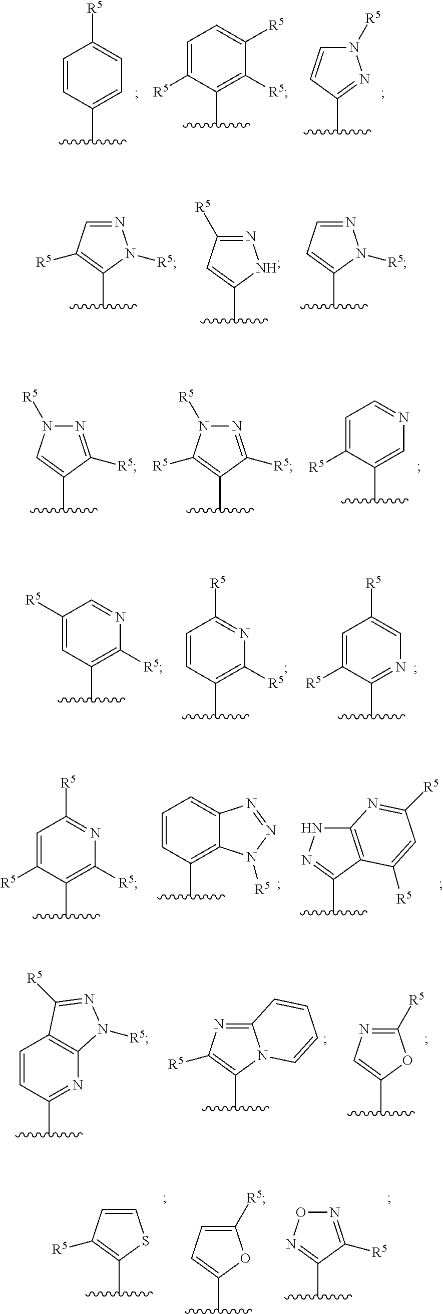

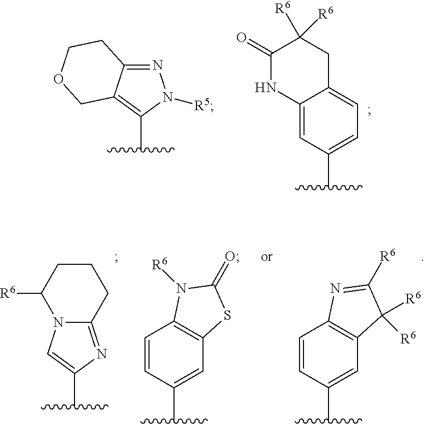

13. The compound of claim 11 or 12, or a pharmaceutically acceptable salt thereof, wherein R³ is represented by the following formula:

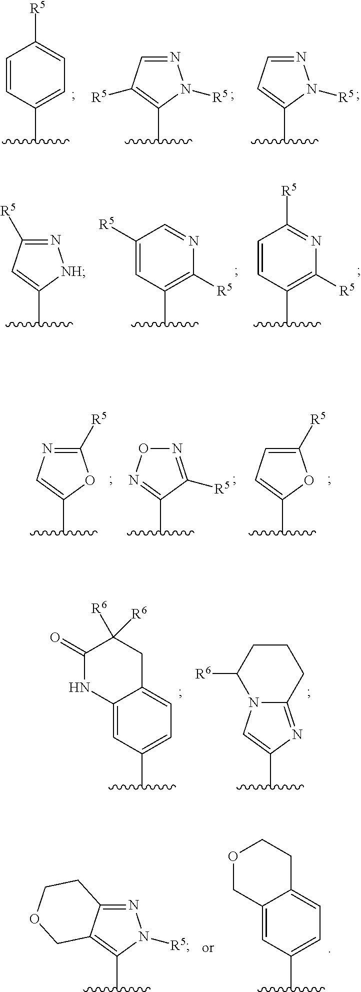

14. The compound of any one of claims 1 to 13, or a pharmaceutically acceptable salt thereof, wherein R⁵, for each occurrence, is independently halo, —OR^{5a}, C₁₋₆alkyl, 4 to 7-membered monocyclic heterocyclyl, or phenyl, wherein the C₁₋₆alkyl is optionally substituted with —OR^{5a} or halo, and wherein the 5- to 6-membered monocyclic heteroaryl is optionally substituted with C₁₋₃alkyl; and R⁶ is C₁₋₃alkyl.

15. The compound of any one of claims 1 to 13, or a pharmaceutically acceptable salt thereof, wherein R⁵, for each occurrence, is independently halo, —OR^{5a}, C₁₋₆alkyl, 4 to 7-membered monocyclic heterocyclyl, or phenyl, wherein the C₁₋₆alkyl is optionally substituted with —OR^{5a}.

16. The compound of claim 14, or a pharmaceutically acceptable salt thereof, wherein R⁵, for each occurrence, is independently —F, —Cl, —CH₃, —CH₂CH₃, —CF₃, —OCHF₂, —OCH₃, —CH₂OCH₃, tetrahydropyranyl, phenyl, pyridinyl; and R⁶ is —CH₃, optionally, wherein R⁵ is represented by the following formula:

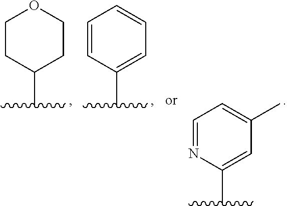

17. The compound of any one of claims 1 to 16, or a pharmaceutically acceptable salt thereof, wherein R¹ and R² together with the nitrogen atom from which they are attached form a 4 to 9 membered heterocyclyl optionally substituted with one to three R⁴.

18. The compound of any one of claims 1 to 16, or a pharmaceutically acceptable salt thereof, wherein R¹ and R² together with the nitrogen atom from which they are attached form a 4 to 6-membered monocyclic saturated heteterocyclyl or a 8 to 9 membered bicyclic heterocyclyl, each of which is optionally substituted with one to three R⁴.

19. The compound of claim 18, or a pharmaceutically acceptable salt thereof, wherein R¹ and R² together with the nitrogen atom from which they are attached form a group represented by the following formula: 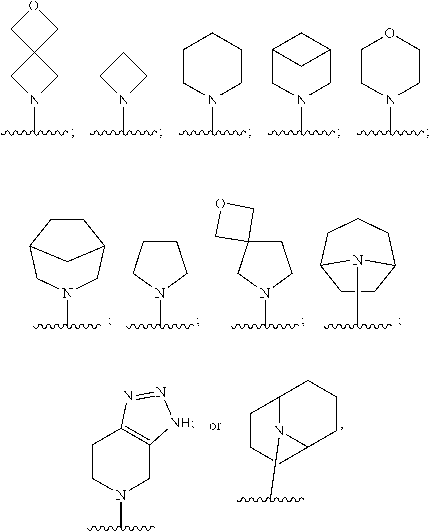
each of which is optionally substituted with one to three R⁴.

20. The compound of claim 18, or a pharmaceutically acceptable salt thereof, wherein R¹ and R² together with the nitrogen atom from which they are attached form a group represented by the following formula: 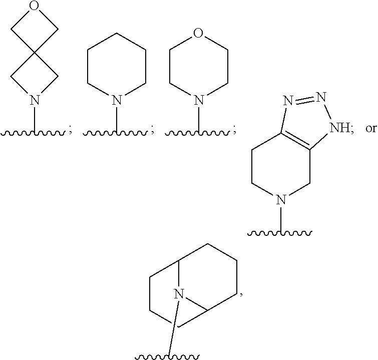
each of which is optionally substituted with one to three R⁴.

21. The compound of claim 19, or a pharmaceutically acceptable salt thereof, wherein R¹ and R² together with the nitrogen atom from which they are attached form a group represented by the following formula:

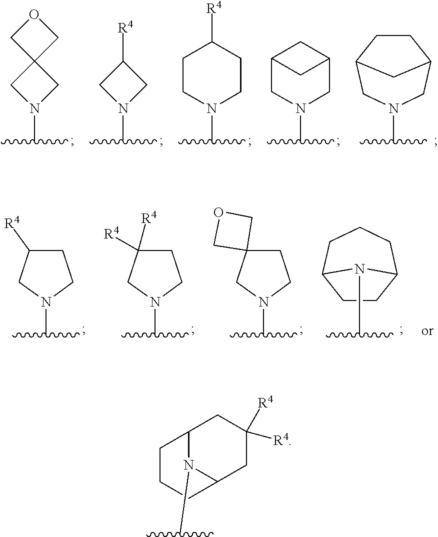

22. The compound of claim 20, or a pharmaceutically acceptable salt thereof, wherein R¹ and R² together with the nitrogen atom from which they are attached form a group represented by the following formula:

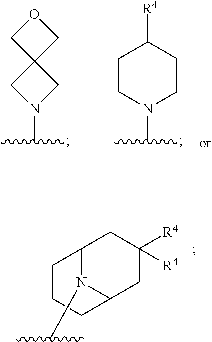

23. The compound of any one of claims 1 to 16, or a pharmaceutically acceptable salt thereof, wherein:
R¹ is Het, —C₁₋₃alkyl-Het, —C₁₋₃alkyl-C₃₋₆cyclolkyl, C₃₋₆cycloalkyl or C₁₋₆alkyl, wherein the C₃₋₆cycloalkyl and the C₁₋₆alkyl are each optionally substituted with one or two R⁴;
R² is H or C₁₋₃alkyl;
Het is a 4 to 6 membered monocyclic saturated heterocyclyl or 6 to 7 membered bicyclic saturated heterocyclyl, wherein the 4 to 6 membered monocyclic saturated heterocyclyl and 6 to 7 membered bicyclic saturated heterocyclyl contain one oxygen ring atom and are each optionally substituted with one or two R⁴; and
R⁴, for each occurrence, is independently —OR^{4a}, halo, or C₁₋₃alkyl, wherein the C₁₋₃alkyl is optionally substituted by —OR^{4a};
R^{4a} is H or C₁₋₃alkyl.

24. The compound of any one of claims 1 to 16, or a pharmaceutically acceptable salt thereof, wherein:
R¹ is Het, —C₁₋₃alkyl-Het, C₃₋₆cycloalkyl or C₁₋₆alkyl, wherein the C₃₋₆cycloalkyl and the C₁₋₆alkyl are each optionally substituted with one or two R⁴;
R² is H or C₁₋₃alkyl;
Het is a 4 to 6 membered monocyclic saturated heterocyclyl or 6 to 7 membered bicyclic saturated heterocyclyl, wherein the 4 to 6 membered monocyclic saturated heterocyclyl and 6 to 7 membered bicyclic saturated heterocyclyl contain one oxygen ring atom and are each optionally substituted with one or two R⁴; and
R⁴, for each occurrence, is independently —OH, halo or C₁₋₃alkyl.

25. The compound of claim 23, or a pharmaceutically acceptable salt thereof, wherein Het is oxetanyl, tetrahydro-2H-pyranyl, tetrahydrofuranyl, 2-oxaspiro[3.3]heptanyl, or 5-oxabicyclo[2.1.1]hexanyl, each of which is optionally substituted with one R⁴.

26. The compound of claim 24, or a pharmaceutically acceptable salt thereof, wherein Het is oxetanyl, tetrahydro-2H-pyranyl, or 5-oxabicyclo[2.1.1]hexanyl, each of which is optionally substituted with one R⁴.

27. The compound of claim 23, or a pharmaceutically acceptable salt thereof, wherein R¹ is represented by the following formula: 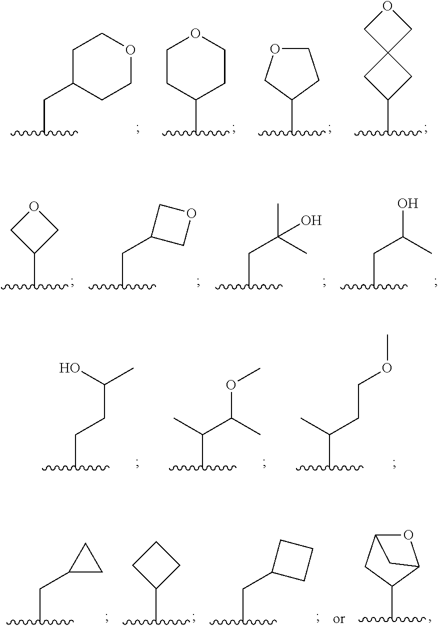
each of which is optionally substituted with one or two R⁴.

28. The compound of claim 24, or a pharmaceutically acceptable salt thereof, wherein R¹ is represented by the following formula: 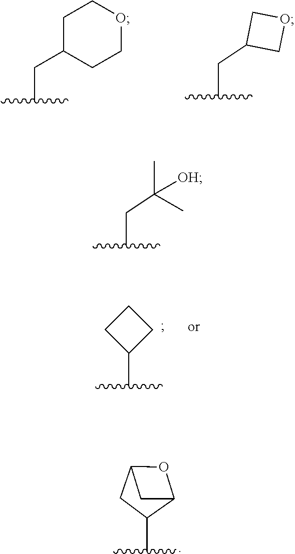
each of which is optionally substituted with one or two R⁴.

29. The compound of claim 23, or a pharmaceutically acceptable salt thereof, wherein R¹ is represented by the following formula:

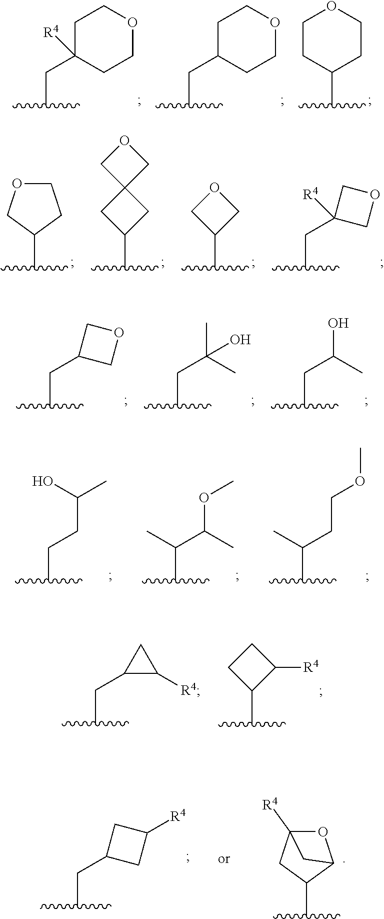

30. The compound of claim 28, or a pharmaceutically acceptable salt thereof, wherein R¹ is represented by the following formula:

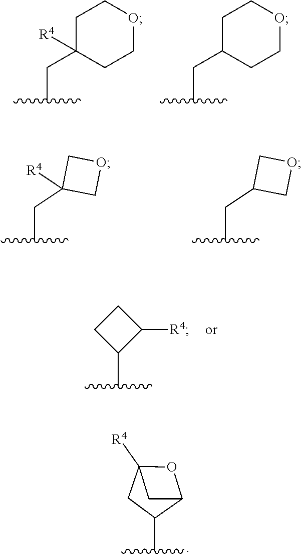

31. The compound of any one of claims 23 to 30, or a pharmaceutically acceptable salt thereof, wherein R² is H or —CH₃.

32. The compound of any one of claims 23 to 30, or a pharmaceutically acceptable salt thereof, wherein R² is H.

33. The compound of any one of claims 1 to 32, or a pharmaceutically acceptable salt thereof, wherein each R⁴, for each occurrence, is independently —F, —CH₃, —CH₂CH₂OCH₃, —CH₂OH, —OH, or —OCH₃.

34. The compound of claim 3, wherein the compound is represented by the Formula (III) or (IV): 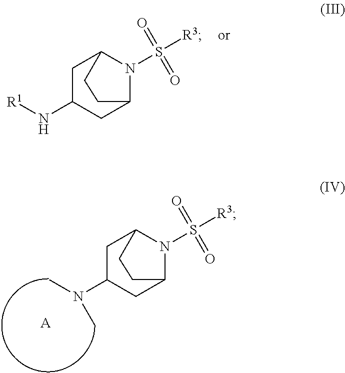
or a pharmaceutically acceptable salt thereof, wherein:
R¹ is C₃₋₆cycloalkyl, Het, or —CH₂-Het, wherein the C₃₋₆cycloalkyl is optionally substituted with one R⁴;
Het is a 4 to 6 membered monocyclic saturated heterocyclyl or a 6 to 7 membered bicyclic saturated heterocyclyl, wherein the 4 to 6 membered monocyclic saturated heterocyclyl and the 6 to 7 membered bicyclic saturated heterocyclyl contain one oxygen ring atom and are each optionally substituted with one R⁴;
Ring A is a 4 to 6 membered monocyclic saturated heterocyclyl or a 8 or 9 membered bicyclic heterocyclyl, each of which is optionally substituted with one or two R⁴;
R³ is phenyl, pyridinyl, or a 5-membered heteroaryl, each of which is optionally substituted with one or two R⁵;
R⁴, for each occurrence, is independently selected from-OH, halo and C₁₋₃alkyl;
R⁵, for each occurrence, is independently selected from halo, —OR^{5a}, C₁₋₆alkyl, 4 to 6-membered monocyclic heterocyclyl, or phenyl, wherein the C₁₋₆alkyl is optionally substituted with one to three halo or —OR^{5a},
or two R⁵ together with their intervening atoms form a 6-membered heterocycle optionally substituted with one to three R⁶ independently selected from C₁₋₆alkyl, oxo, and halo; and
R^{5a} is H, C₁₋₃alkyl, or C₁₋₃haloalkyl.

35. The compound of claim 34, or a pharmaceutically acceptable salt thereof, wherein:
Het is oxetanyl, tetrahydro-2H-pyranyl, or 5-oxabicyclo[2.1.1]hexanyl, each of which is optionally substituted with one R⁴;
Ring A is 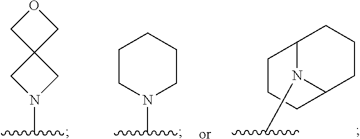
each of which is optionally substituted with one to two R⁴;
R³ is 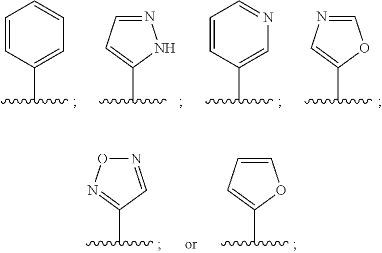
each of which is optionally substituted with one or two R⁵;
or R³ is 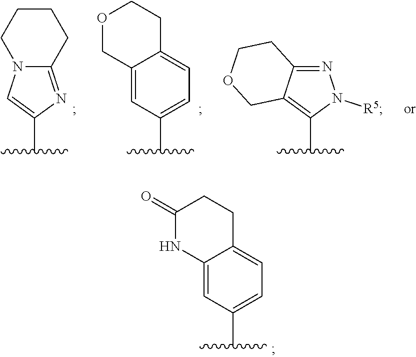
each of which is optionally substituted with one or two R⁶.

36. The compound of claim 35, or a pharmaceutically acceptable salt thereof, wherein R³ is represented by the following formula:

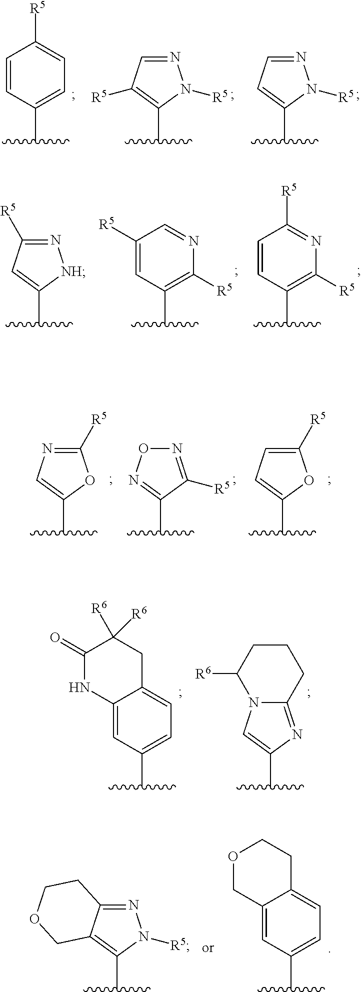

37. The compound of any one of claims 34 to 36, or a pharmaceutically acceptable salt thereof, wherein Het is represented by the following formula:

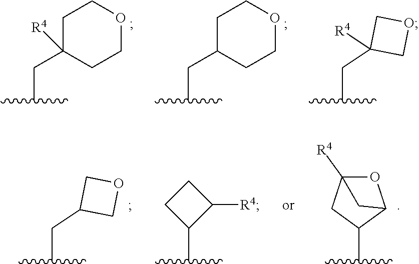

38. The compound of any one of claims 34 to 37, or a pharmaceutically acceptable salt thereof, wherein R⁴, for each occurrence, is independently —CH₃, —OH, or F.

39. The compound of any one of claims 34 to 38, or a pharmaceutically acceptable salt thereof, wherein R⁵, for each occurrence, is independently —OCF₃, —OCHF₂, Cl, —CH₂OCH₃, —CH₃, —CF₃, —OCH₃, —CH₂CH₃,

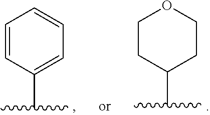 [SMILES: *c1ccccc1 ; *C1CCOCC1]

40. The compound of claim 1 or 2, wherein the compound is represented by Formula V: 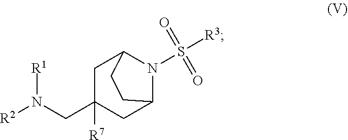
or a pharmaceutically acceptable salt thereof.

41. The compound of claim 40, wherein the compound is represented by Formulas (VA) or (VB): 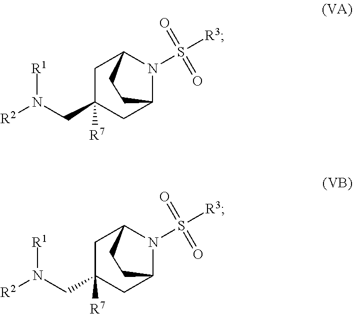
or a pharmaceutically acceptable salt thereof.

42. The compound of claims 40 to 41, or a pharmaceutically acceptable salt thereof, wherein R³ is selected from the group consisting of phenyl, pyrazolyl, isoxazoyl, and pyridinyl, each of which is optionally substituted with one to three R⁵.

43. The compound of any one of claims 40 to 42, or a pharmaceutically acceptable salt thereof, wherein R³ is represented by the following formula: 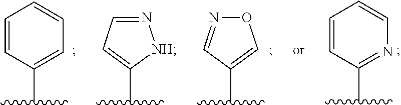
wherein each of the formula depicted above is optionally substituted with one to three R⁵.

44. The compound of any one of claims 40 to 43, or a pharmaceutically acceptable salt thereof, wherein R³ is represented by the following formula:

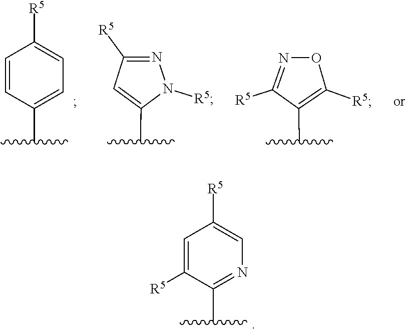

45. The compound of any one of claims 40 to 44, or a pharmaceutically acceptable salt thereof, wherein R⁵, for each occurrence, is independently halo, —OR^{5a} or C₁₋₆alkyl.

46. The compound of any one of claims 40 to 45, wherein R⁷ is H or OH.

47. The compound of any one of claims 40 to 46, or a pharmaceutically acceptable salt thereof, wherein R¹ and R² together with the nitrogen atom from which they are attached form a group represented by the following formula:

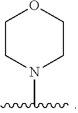 [SMILES: *N1CCOCC1]

48. The compound of claim 3, or a pharmaceutically acceptable salt thereof, wherein the compound is represented by the Formula (VI) or (VII): 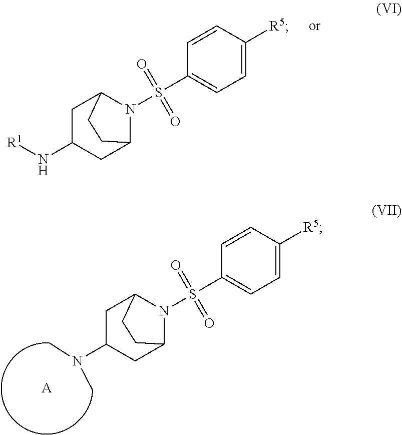
or a pharmaceutically acceptable salt thereof, wherein:
R¹ is C₁₋₆alkyl, C₃₋₆cycloalkyl, Het, —CH₂-C₃₋₄cycloalkyl, or —CH₂-Het, wherein the C₁₋₆alkyl, C₃₋₆cycloalkyl, and —CH₂—C₃₋₄cycloalkyl are each optionally substituted with one or two R⁴;
Het is a 4 to 6 membered monocyclic saturated heterocyclyl containing one oxygen ring atom;
Ring A is a 4 to 6 membered monocyclic saturated heterocyclyl or a 8 or 9 membered bicyclic saturated heterocyclyl, each of which is optionally substituted with one or two R⁴;
R⁴, for each occurrence, is independently selected from-OR^{4a}, —CH₂—OR^{4a}, and C₁₋₃alkyl;
R^{4a} is H or C₁₋₃alkyl;
R⁵ is —OR^{5a};
R^{5a} is C₁₋₃haloalkyl.

49. The compound of claim 48, or a pharmaceutically acceptable salt thereof, wherein Het is tetrahydropyranyl or oxetanyl.

50. The compound of claim 48 or 49, or a pharmaceutically acceptable salt thereof, wherein R¹ is
(i) C₁₋₆alkyl optionally substituted with one or two R⁴;
(ii) represented by the following formula: or 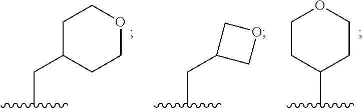
(iii) represented by the following formula: 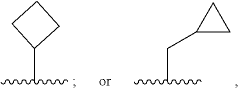 [SMILES: *C1CCC1 ; *CC1CC1]
each of which is optionally substituted with one or two R⁴.

51. The compound of claim 50, or a pharmaceutically acceptable salt thereof, wherein R¹ is —CH(CH₃)CH₂CH₂OCH₃ or CH₂C(CH₃)₂OH, or R¹ is represented by the following formula

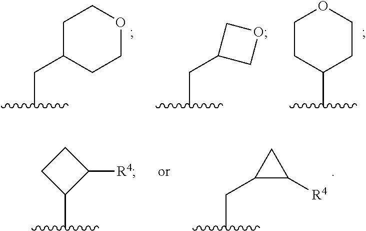

52. The compound of claim 48, or a pharmaceutically acceptable salt thereof, wherein Ring A is 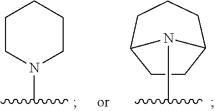 [SMILES: *N1CCCCC1 ; *N1C2CCCC1CC2]
each of which is optionally substituted with one to two R⁴.

53. The compound of claim 52, or a pharmaceutically acceptable salt thereof, wherein Ring A is

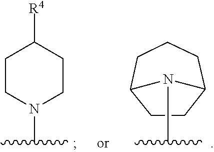

54. The compound of any one of claims 48 to 53, or a pharmaceutically acceptable salt thereof, wherein each R⁴ is independently —OH, —OCH₃, —CH₂OH, or —CH₃.

55. The compound of any one of claims 48 to 54, or a pharmaceutically acceptable salt thereof, wherein R⁵ is —OCHF₂.

56. The compound of claim 1, wherein the compound is:
3-Methyl-4-(((1R,3r,5S)-3-(4-methylpiperidin-1-yl)-8-azabicyclo[3.2.1]octan-8-yl)sulfonyl)-1,2,5-oxadiazole;
5-(((1R,3r,5S)-3-(4-Methylpiperidin-1-yl)-8-azabicyclo[3.2.1]octan-8-yl)sulfonyl)-2-(tetrahydro-2H-pyran-4-yl) oxazole;
(1R,3s,5S)-8-((2-Methoxy-6-methylpyridin-3-yl)sulfonyl)-3-(4-methylpiperidin-1-yl)-8-azabicyclo[3.2.1]octane;
(1R,3s,5S)-8-((4-Chloro-1-(methoxymethyl)-1H-pyrazol-5-yl)sulfonyl)-3-(4-methylpiperidin-1-yl)-8-azabicyclo[3.2.1]octane;
3,3-Dimethyl-6-(((1R,3s,5S)-3-(4-methylpiperidin-1-yl)-8-azabicyclo[3.2.1]octan-8-yl)sulfonyl)-3,4-dihydroquinolin-2 (1H)-one;
5-(((1R,3s,5S)-3-(4-Methylpiperidin-1-yl)-8-azabicyclo[3.2.1]octan-8-yl)sulfonyl)-2-(tetrahydro-2H-pyran-4-yl) oxazole;
(1R,3s,5S)-8-(Isochroman-7-ylsulfonyl)-3-(4-methylpiperidin-1-yl)-8-azabicyclo[3.2.1]octane;
(1R,3s,5S)-8-((5-(Methoxymethyl) furan-2-yl)sulfonyl)-3-(4-methylpiperidin-1-yl)-8-azabicyclo[3.2.1]octane;
2-Methyl-3-(((1R,3s,5S)-3-(4-methylpiperidin-1-yl)-8-azabicyclo[3.2.1]octan-8-yl)sulfonyl)-2,4,6,7-tetrahydropyrano[4,3-c]pyrazole;
(1R,3s,5S)-3-(4-Methylpiperidin-1-yl)-8-((3-phenyl-1H-pyrazol-5-yl)sulfonyl)-8-azabicyclo[3.2.1]octane;
5-Methyl-2-(((1R,3s,5S)-3-(4-methylpiperidin-1-yl)-8-azabicyclo[3.2.1]octan-8-yl)sulfonyl)-5,6,7,8-tetrahydroimidazo[1,2-a]pyridine;
(1R,3s,5S)-8-((1,4-Dimethyl-1H-pyrazol-5-yl)sulfonyl)-3-(4-methylpiperidin-1-yl)-8-azabicyclo[3.2.1]octane;
(1R,3s,5S)-8-((1-Ethyl-1H-pyrazol-5-yl)sulfonyl)-3-(4-methylpiperidin-1-yl)-8-azabicyclo[3.2.1]octane;
2-((8-((4-(Difluoromethoxy)phenyl)sulfonyl)-8-azabicyclo[3.2.1]octan-3-yl)amino)cyclobutan-1-ol;
8-((4-(Difluoromethoxy)phenyl)sulfonyl)-N-((3-methyloxetan-3-yl)methyl)-8-azabicyclo[3.2.1]octan-3-amine;
9-(8-((4-(Difluoromethoxy)phenyl)sulfonyl)-8-azabicyclo[3.2.1]octan-3-yl)-3,3-difluoro-9-azabicyclo[3.3.1]nonane;
4-((((1R,3r,5S)-8-((4-(Difluoromethoxy)phenyl)sulfonyl)-8-azabicyclo[3.2.1]octan-3-yl)amino)methyl)tetrahydro-2H-pyran-4-ol;
4-((((1R,3s,5S)-8-((4-(Difluoromethoxy)phenyl)sulfonyl)-8-azabicyclo[3.2.1]octan-3-yl)amino)methyl)tetrahydro-2H-pyran-4-ol;
8-((4-(Difluoromethoxy)phenyl)sulfonyl)-N-((tetrahydro-2H-pyran-4-yl)methyl)-8-azabicyclo[3.2.1]octan-3-amine;
(1R,3r,5S)-8-((4-(Difluoromethoxy)phenyl)sulfonyl)-3-(4-methylpiperidin-1-yl)-8-azabicyclo[3.2.1]octane;
(1R,3s,5S)-8-((4-(Difluoromethoxy)phenyl)sulfonyl)-3-(4-methylpiperidin-1-yl)-8-azabicyclo[3.2.1]octane;
6-((1R,3s,5S)-8-((2-Methyl-6-(trifluoromethyl)pyridin-3-yl)sulfonyl)-8-azabicyclo[3.2.1]octan-3-yl)-2-oxa-6-azaspiro[3.3]heptane;
6-((1R,3r,5S)-8-((2-Methyl-6-(trifluoromethyl)pyridin-3-yl)sulfonyl)-8-azabicyclo[3.2.1]octan-3-yl)-2-oxa-6-azaspiro[3.3]heptane;
(1R,3s,5S)-8-((2-Methyl-6-(trifluoromethyl)pyridin-3-yl)sulfonyl)-N-((tetrahydro-2H-pyran-4-yl)methyl)-8-azabicyclo[3.2.1]octan-3-amine;
(1R,3r,5S)-8-((2-Methyl-6-(trifluoromethyl)pyridin-3-yl)sulfonyl)-N-((tetrahydro-2H-pyran-4-yl)methyl)-8-azabicyclo[3.2.1]octan-3-amine;
(1R,3s,5S)-8-((2-Methyl-6-(trifluoromethyl)pyridin-3-yl)sulfonyl)-N-((3-methyloxetan-3-yl)methyl)-8-azabicyclo[3.2.1]octan-3-amine;
(1R,3r,5S)-8-((2-Methyl-6-(trifluoromethyl)pyridin-3-yl)sulfonyl)-N-((3-methyloxetan-3-yl)methyl)-8-azabicyclo[3.2.1]octan-3-amine;
(1R,3s,5S)-8-((4-(Difluoromethoxy)phenyl)sulfonyl)-N-((3-methyloxetan-3-yl)methyl)-8-azabicyclo[3.2.1]octan-3-amine;
(1R,3s,5S)-8-((4-(Difluoromethoxy)phenyl)sulfonyl)-N-((tetrahydro-2H-pyran-4-yl)methyl)-8-azabicyclo[3.2.1]octan-3-amine;
8-((4-(Difluoromethoxy)phenyl)sulfonyl)-N-(1-methyl-2-oxabicyclo[2.1.1]hexan-4-yl)-8-azabicyclo[3.2.1]octan-3-amine;
(1R,3s,5S)-8-((4-(Difluoromethoxy)phenyl)sulfonyl)-N-(oxetan-3-ylmethyl)-8-azabicyclo[3.2.1]octan-3-amine;
(1R,3r,5S)-8-((4-(Difluoromethoxy)phenyl)sulfonyl)-N-((tetrahydro-2H-pyran-4-yl)methyl)-8-azabicyclo[3.2.1]octan-3-amine;
(1R,3r,5S)-8-((4-(Difluoromethoxy)phenyl)sulfonyl)-3-(morpholinomethyl)-8-azabicyclo[3.2.1]octan-3-ol;
(1R,3s,5S)-8-((4-(Difluoromethoxy)phenyl)sulfonyl)-3-(morpholinomethyl)-8-azabicyclo[3.2.1]octan-3-ol;
4-(((1R,3s,5S)-8-((4-(Difluoromethoxy)-2,6-difluorophenyl)sulfonyl)-8-azabicyclo[3.2.1]octan-3-yl)methyl)morpholine;
4-(((1R,3s,5S)-8-((1,3-Dimethyl-1H-pyrazol-5-yl)sulfonyl)-8-azabicyclo[3.2.1]octan-3-yl)methyl)morpholine;
4-(((1R,3r,5S)-8-((1,3-Dimethyl-1H-pyrazol-5-yl)sulfonyl)-8-azabicyclo[3.2.1]octan-3-yl)methyl)morpholine;
4-(((1R,3s,5S)-8-((3,5-Dimethylisoxazol-4-yl)sulfonyl)-8-azabicyclo[3.2.1]octan-3-yl)methyl)morpholine;
4-(((1R,3s,5S)-8-((4,6-Dimethylpyridin-3-yl)sulfonyl)-8-azabicyclo[3.2.1]octan-3-yl)methyl)morpholine;
(1R,3r,5S)-3-(4-Methylpiperidin-1-yl)-8-((1-(4-methylpyridin-2-yl)-1H-pyrazol-4-yl)sulfonyl)-8-azabicyclo[3.2.1]octane;
(1R,3s,5S)-3-(4-Methylpiperidin-1-yl)-8-((1-(4-methylpyridin-2-yl)-1H-pyrazol-4-yl)sulfonyl)-8-azabicyclo[3.2.1]octane;
4-(((1R,3s,5S)-3-(4-Methylpiperidin-1-yl)-8-azabicyclo[3.2.1]octan-8-yl)sulfonyl)-2-(trifluoromethyl) thiazole;
(1R,3s,5S)-8-((2,6-Difluoro-4-methoxyphenyl)sulfonyl)-3-(4-methylpiperidin-1-yl)-8-azabicyclo[3.2.1]octane;
(1R,3s,5S)-3-(4-Methylpiperidin-1-yl)-8-((1-phenyl-1H-1,2,3-triazol-4-yl)sulfonyl)-8-azabicyclo[3.2.1]octane;
1,3-Dimethyl-5-(((1R,3s,5S)-3-(4-methylpiperidin-1-yl)-8-azabicyclo[3.2.1]octan-8-yl)sulfonyl)-1H-pyrazolo[3,4-b]pyridine;
(1R,3s,5S)-8-((3,5-Dimethylpyridin-2-yl)sulfonyl)-3-(4-methylpiperidin-1-yl)-8-azabicyclo[3.2.1]octane;
(1R,3s,5S)-8-((2,6-Dimethylpyridin-3-yl)sulfonyl)-3-(4-methylpiperidin-1-yl)-8-azabicyclo[3.2.1]octane;
1-((8-((4-(Difluoromethoxy)phenyl)sulfonyl)-8-azabicyclo[3.2.1]octan-3-yl)amino) propan-2-ol;
1-(8-((4-(Difluoromethoxy)phenyl)sulfonyl)-8-azabicyclo[3.2.1]octan-3-yl)-3-methylpyrrolidin-3-ol;
8-((4-(Difluoromethoxy)phenyl)sulfonyl)-3-(3-methoxypyrrolidin-1-yl)-8-azabicyclo[3.2.1]octane;
6-(8-((4-(Difluoromethoxy)phenyl)sulfonyl)-8-azabicyclo[3.2.1]octan-3-yl)-2-oxa-6-azaspiro[3.4]octane;
8-((4-(Difluoromethoxy)phenyl)sulfonyl)-N-(4-methoxybutan-2-yl)-8-azabicyclo[3.2.1]octan-3-amine;
8-((4-(Difluoromethoxy)phenyl)sulfonyl)-8,8′-diaza-3,8′-bi(bicyclo[3.2.1]octane);
8-((4-(Difluoromethoxy)phenyl)sulfonyl)-3-(3-(2-methoxyethyl) azetidin-1-yl)-8-azabicyclo[3.2.1]octane;
8-((4-(Difluoromethoxy)phenyl)sulfonyl)-N-(3-methoxybutan-2-yl)-8-azabicyclo[3.2.1]octan-3-amine;
rac-((1R,2S)-2-(((8-((4-(Difluoromethoxy)phenyl)sulfonyl)-8-azabicyclo[3.2.1]octan-3-yl)amino)methyl)cyclopropyl)methanol;
3-(8-((4-(Difluoromethoxy)phenyl)sulfonyl)-8-azabicyclo[3.2.1]octan-3-yl)-3-azabicyclo[3.1.1]heptane;
8′-((4-(Difluoromethoxy)phenyl)sulfonyl)-3,8′-diaza-3,3′-bi(bicyclo[3.2.1]octane);
rac-4-(((1R,3r,5S)-8-((4-(Difluoromethoxy)phenyl)sulfonyl)-8-azabicyclo[3.2.1]octan-3-yl)amino)butan-2-ol;
rac-4-(((1R,3s,5S)-8-((4-(Difluoromethoxy)phenyl)sulfonyl)-8-azabicyclo[3.2.1]octan-3-yl)amino)butan-2-ol;
(1R,3r)-3-((((1R,3s,5S)-8-((4-(Difluoromethoxy)phenyl)sulfonyl)-8-azabicyclo[3.2.1]octan-3-yl)amino)methyl)cyclobutan-1-ol;
(1R,3r)-3-((((1R,3r,5S)-8-((4-(Difluoromethoxy)phenyl)sulfonyl)-8-azabicyclo[3.2.1]octan-3 yl)amino)methyl)cyclobutan-1-ol;
(1S,3s)-3-((((1R,3s,5S)-8-((4-(Difluoromethoxy)phenyl)sulfonyl)-8-azabicyclo[3.2.1]octan-3-yl)amino)methyl)cyclobutan-1-ol;
(1S,3s)-3-((((1R,3r,5S)-8-((4-(Difluoromethoxy)phenyl)sulfonyl)-8-azabicyclo[3.2.1]octan-3-yl)amino)methyl)cyclobutan-1-ol;
(1R,3r,5S)-8-((4-(Difluoromethoxy)phenyl)sulfonyl)-N-methyl-N-(tetrahydro-2H-pyran-4-yl)-8-azabicyclo[3.2.1]octan-3-amine;
(1R,3s,5S)-8-((4-(Difluoromethoxy)phenyl)sulfonyl)-N-methyl-N-(tetrahydro-2H-pyran-4-yl)-8-azabicyclo[3.2.1]octan-3-amine;
4-(((1R,3s,5S)-8-((4-(Difluoromethoxy)phenyl)sulfonyl)-8-azabicyclo[3.2.1]octan-3-yl)methyl)morpholine;
(1R,3s,5S)-8-((2-Methyl-6-(trifluoromethyl)pyridin-3-yl)sulfonyl)-N-(2-oxaspiro[3.3]heptan-6-yl)-8-azabicyclo[3.2.1]octan-3-amine
(1R,3r,5S)-8-((2-Methyl-6-(trifluoromethyl)pyridin-3-yl)sulfonyl)-N-(2-oxaspiro[3.3]heptan-6-yl)-8-azabicyclo[3.2.1]octan-3-amine;
8-((4-(Difluoromethoxy)phenyl)sulfonyl)-N—((R)-tetrahydrofuran-3-yl)-8-azabicyclo[3.2.1]octan-3-amine;
(1R,3r,5S)-8-((4-(Difluoromethoxy)phenyl)sulfonyl)-N-((3-fluorooxetan-3-yl)methyl)-8-azabicyclo[3.2.1]octan-3-amine;
(1R,3r,5S)-8-((4-(Difluoromethoxy)phenyl)sulfonyl)-N-(oxetan-3-ylmethyl)-8-azabicyclo[3.2.1]octan-3-amine;
8-((4-(Difluoromethoxy)phenyl)sulfonyl)-N-(tetrahydro-2H-pyran-4-yl)-8-azabicyclo[3.2.1]octan-3-amine;
8-((4-(Difluoromethoxy)phenyl)sulfonyl)-N-(oxetan-3-yl)-8-azabicyclo[3.2.1]octan-3-amine;
(1R,3s,5S)-8-((2-Methyl-6-(trifluoromethyl)pyridin-3-yl)sulfonyl)-N-(tetrahydro-2H-pyran-4-yl)-8-azabicyclo[3.2.1]octan-3-amine;
1-(((1R,3s,5S)-8-((4-(Difluoromethoxy)phenyl)sulfonyl)-8-azabicyclo[3.2.1]octan-3-yl)amino)-2-methylpropan-2-ol; and
1-(((1R,3r,5S)-8-((4-(Difluoromethoxy)phenyl)sulfonyl)-8-azabicyclo[3.2.1]octan-3-yl)amino)-2-methylpropan-2-ol;
or a pharmaceutically acceptable salt thereof.

57. A pharmaceutical composition comprising a compound of any one of claims 1 to 56, or a pharmaceutically acceptable salt thereof, and a pharmaceutically acceptable excipient.

58. A method of treating a disease or disorder mediated by Emopamil-Binding Protein comprising administering to a subject an effective amount of a compound of any one of claims 1 to 56, or a pharmaceutically acceptable salt thereof, or the pharmaceutical composition of claim 57.

59. A method of treating an autoimmune disease in a subject comprising administering to the subject an effective amount of a compound of any one of claims 1 to 56, or a pharmaceutically acceptable salt thereof, or the pharmaceutical composition of claim 57.

60. The method of claim 59, wherein said autoimmune disease is multiple sclerosis.

61. The method of claim 60, wherein said compound or pharmaceutical composition repairs or forms new myelin sheaths in said subject.

62. A method of promoting myelination in a subject with a myelin-related disease or disorder comprising administering to the subject an effective amount of a compound of any one of claims 1 to 56, or a pharmaceutically acceptable salt thereof, or the pharmaceutical composition of claim 57.
